(19) United States
(12) Patent Application Publication
(10) Pub. No.: US 2025/0157622 A1
(43) Pub. Date: May 15, 2025

(54) CLOUD-BASED AI-ENHANCED PHOTOBIOMODULATION WELLNESS SYSTEM WITH INTEGRATED VIRTUAL AND DISTRIBUTED LEDGER FEATURES

(71) Applicant: Reversal Solutions, Inc., Belmont, CA (US)

(72) Inventors: John A Fortkort, Austin, TX (US); Robert R. Mullen, III, Woodland Hills, CA (US); Thomas A. Cocotis, Huntington Beach, CA (US); Peter A. Newsom, Woodside, CA (US)

(21) Appl. No.: 19/007,480

(22) Filed: Jan. 1, 2025

Related U.S. Application Data
(63) Continuation of application No. 18/228,651, filed on Jul. 31, 2023.
(60) Provisional application No. 63/525,706, filed on Jul. 9, 2023.
(60) Provisional application No. 63/393,849, filed on Jul. 30, 2022.

(51) Int. Cl. G16H 20/40; G06N 3/0442; G06N 3/08

(52) U.S. Cl. CPC G16H 20/40; G06N 3/0442; G06N 3/08

(57) ABSTRACT

A method is provided for coordinated wellness treatments using a plurality of PBM devices. The method includes collecting wellness data from each PBM device; transmitting the collected wellness data to a server; analyzing the wellness data to generate personalized treatment recommendations; recording the wellness data and treatment recommendations onto a distributed ledger system; communicating the personalized treatment recommendations to each PBM device; and adjusting the PBM treatments based on the personalized recommendations.

### CROSS-REFERENCE TO RELATED APPLICATION

This application is a continuation of U.S. Ser. No. 18/228,651, filed Jul. 31, 2023, having the same inventorship and entitled “SUBSCRIPTION-BASED PHOTOBIOMODULATION WELLNESS SYSTEM WITH COMMUNITY OF USERS”, which claims the benefit of priority from U.S. provisional application No. 63/525,706, filed Jul. 9, 2023, having the same inventorship and the same title, and which is incorporated herein by reference in its entirety, and which also claims the benefit of priority from U.S. provisional application No. 63/393,849, filed Jul. 30, 2022, having the same inventorship and entitled “CLOSED LOOP ARTIFICIAL INTELLIGENCE WELLNESS OPTIMIZATION SYSTEM”, and which is incorporated herein by reference in its entirety.

### FIELD OF THE DISCLOSURE

The present application relates generally to photobiomodulation (PBM), and more specifically to an AI-augmented PBM wellness system having a community of users.

### BACKGROUND OF THE DISCLOSURE

Photobiomodulation (PBM) is a type of light therapy that utilizes non-ionizing electromagnetic energy to trigger photochemical changes in cellular structures that are receptive to photons. Various devices have been developed in the art to implement PBM or processes related thereto. Examples of such devices are described, for example, in U.S. 2019/0246463A1 (Williams et al.)., U.S. US2019/0175936 (Gretz et al.), WO2019/053625 (Lim), U.S. U.S. 2014/0243933 (Ginggen), U.S. 2019/0142636 (Tedford et al.), U.S. Pat. No. 7,354,432 (Eells et al.), U.S. 2008/0091249 (Wang), U.S. Pat. No. 10,391,330 (Bourke et al.) and U.S. 2016/0129278 (Mayer).

Although the effects of PBM are not fully understood, the underlying physiological processes at play during PBM have been the subject of considerable research. Mitochondria are believed to be central to these processes. These intracellular organelles generate adenosine triphosphate (ATP), which is the main source energy for cellular activity and metabolism.

Mitochondria absorb visible red and near infrared light (NIR) energy at the cellular level and utilize the absorbed radiation to produce cellular energy in the form of ATP. A mitochondrial enzyme (cytochrome oxidase c) is believed to be central to this process. This enzyme is a chromophore, and accepts photonic energy of specific wavelengths.

The process utilized by mitochondria to generate ATP also creates reactive oxygen species (ROS). These species promote gene transcription, cellular repair and healing. This process is also believed to release nitric oxide back into the body. Nitric oxide helps cells to communicate with each other, and also improves blood circulation and dilates blood vessels.

It has recently been demonstrated that PBM has anti-inflammatory effects which are mediated by cytokines. Thus, Shamloo et al. [Shamloo S. Defensor E, Clari P, Ogawa O, Vidano L, Lin J S. Fortkort J A, Shamloo M, Barron A E. The anti-inflammatory effects of photobiomodulation are mediated by cytokines: Evidence from a mouse model of inflammation. Front Neurosci. 2023 Apr. 6:17:1150156. doi: 10.3389/fins.2023.1150156. PMID: 37090796: PMCID: PMC10115964] showed that PBM downregulates LPS induction of key proinflammatory cytokines associated with inflammasome activation and upregulates anti-inflammatory cytokines. These results demonstrate the potential of PBM as an anti-inflammatory treatment that acts through cytokine expression modulation.

PBM has also emerged as a potential therapeutic for treating or preventing Alzheimer's Disease. Thus, several clinical studies and experiments have demonstrated that PBM can have a positive effect on AD treatment or prevention. For example, irradiation with red and near-infrared light at a low dose is found to reduce the accumulation of amyloid-β (AB) plaques in the central nervous system.

### BRIEF DESCRIPTION OF THE DRAWINGS

FIG. 1 is an illustration of a closed-loop AI wellness optimization system in accordance with the teachings herein.

FIG. 2 is an illustration of the software client interface in the system of FIG. 1.

FIG. 3 is an illustration of the server side administrative interface in the system of FIG. 1.

FIG. 4 is an illustration of a variation of the embodiment of FIG. 1 which incorporates an augmented reality (AR) device therein.

FIG. 5 is a simplified illustration of a possible 3D model produced by the embodiment of FIG. 4.

FIGS. 6-9 are illustrations of an embodiment of a photobiomodulation device which may be utilized in the system of FIG. 1.

FIG. 10 is an illustration of a particular, nonlimiting embodiment of a web-based subscription service of the type disclosed herein.

FIG. 11 is an illustration of a particular, nonlimiting embodiment of a business ecosystem that may develop around a subscription-based service of the type disclosed herein.

FIG. 12 depicts the architecture of a containerized system of the type disclosed herein.

FIG. 13 depicts the layer architecture of a system for implementing a container-based microservices architecture.

FIG. 14 is an illustration of the features of a Linux operating system adapted for use with the PBM-based wellness systems disclosed herein.

### SUMMARY OF THE DISCLOSURE

In one aspect, a system is provided for promoting wellness. The system comprises a software client, an instance of which is installed on a plurality of client photobiomodulation (PBM) devices, wherein each client PBM device is associated with one of a plurality of users; a wellness database containing wellness data collected from said plurality of users; and a server, in communication with said plurality of client PBM devices, which receives wellness data and treatment objectives from said plurality of users via said software client, and which provides light therapy treatment recommendations to said plurality of client PBM devices; wherein said server is equipped with an artificial intelligence engine which accepts treatment objectives from each of said plurality of users and operates on said wellness database to generate PBM recommendations for each of said plurality of users.

In another aspect, a method is provided for promoting wellness. The method comprises installing a software client on a plurality of client photobiomodulation (PBM) devices, each of which is associated with one of a plurality of users; collecting wellness data from said plurality of users to form a wellness database; receiving, at a server in communication with said plurality of client PBM devices, wellness data and treatment objectives from said plurality of users via said software client; providing light therapy treatment recommendations to said plurality of client PBM devices from the server; accepting treatment objectives from each of said plurality of users by an artificial intelligence engine equipped within said server; and operating on said wellness database by said artificial intelligence engine to generate PBM recommendations for each of said plurality of users.

In a further aspect, a server-side administrative interface for a system promoting wellness. The server side interface comprises a dashboard configured to provide an overview of the system performance and user activity; a user management module configured to allow administrators to add, remove, or modify user accounts; a device management module configured to allow administrators to view and manage photobiomodulation devices connected to the system; a wellness database interface configured to provide access to wellness data and enable data visualization and analysis; an artificial intelligence engine interface configured to provide access to an artificial intelligence engine, allowing training and validation of artificial intelligence models; and a system monitoring module configured to provide tools for monitoring the performance and health of the system.

In still another aspect, a client side user interface for a system promoting wellness is provided. The client side interface comprises an input module configured to allow users to input their treatment objectives; a data display module configured to provide a visual representation of the user's wellness data; a recommendation module configured to display photobiomodulation treatment recommendations; and a device interaction module configured to allow the user to interact with their associated photobiomodulation device.

In a further aspect, a system is provided for promoting wellness. The system comprises a plurality of photobiomodulation (PBM) devices, each associated with a respective user; a server equipped with an artificial intelligence engine; a software client installed on each of the PBM devices or on a user device in communication with one of the PBM devices, the software client including a client-side user interface having an input module for the user to input treatment objectives, a data display module to visualize wellness data, a recommendation module to display PBM treatment recommendations, and a device interaction module to control the PBM device; a server-side administrative interface for monitoring and managing the system; and a wellness database that contains the wellness data collected from the plurality of users, wherein the artificial intelligence engine operates on the wellness data to generate the PBM recommendations.

In another aspect, a wellness promotion system is provided which comprises a photobiomodulation (PBM) device; a virtual reality (VR) headset; a server; and a software client, wherein said software client is installed on said PBM device and said VR headset, and communicates wellness data and treatment objectives to said server; wherein said server receives wellness data and treatment objectives from said software client, and provides light therapy treatment recommendations to said PBM device and VR content recommendations to said VR headset; and wherein said server is equipped with an artificial intelligence (AI) engine which operates on a wellness database to generate PBM recommendations and VR content recommendations based on said wellness data and treatment objectives.

In still another aspect, a method is provided for promoting wellness in a system comprising a photobiomodulation (PBM) device and a virtual reality (VR) headset. The method comprises receiving a user's wellness data and treatment objectives; utilizing an artificial intelligence (AI) engine to analyze the user's wellness data and formulate personalized PBM and VR therapy recommendations; delivering the personalized PBM therapy via the PBM device; and synchronizing the delivery of VR content with the PBM therapy to enhance user experience and therapy effectiveness.

In yet another aspect, a wellness promotion system is provided which comprises a plurality of photobiomodulation (PBM) devices, wherein each of the plurality of PBM devices in in communication with at least one other PBM device, and wherein the system is adapted to use information from the communication between the PBM devices to tailor treatment recommendations for a user.

In another aspect, a method is provided for promoting wellness. The method comprises initiating communication between a plurality of photobiomodulation (PBM) devices, each associated with a user; collecting wellness data from each of the PBM devices; and using the wellness data from the plurality of PBM devices to tailor treatment recommendations for the user.

In a further aspect, a cloud-based wellness system is provided which comprises a plurality of Photobiomodulation (PBM) devices, each device associated with a user and capable of communication with other PBM devices; a distributed ledger system containing records of wellness data and treatment regimens, where each record is cryptographically secure, and any modification is time-stamped and appended to the ledger; a server, equipped with a software client to facilitate communication between PBM devices and to record wellness data and treatment regimens to the distributed ledger system; and at least one processor that operates on the wellness database to generate personalized treatment recommendations, wherein said recommendations are secured and recorded onto the distributed ledger system.

In another aspect, a method is provided for providing coordinated wellness treatments using a plurality of PBM devices. The method comprises collecting wellness data from each PBM device; transmitting the collected wellness data to a server; analyzing the wellness data to generate personalized treatment recommendations; recording the wellness data and treatment recommendations onto a distributed ledger system; communicating the personalized treatment recommendations to each PBM device; and adjusting the PBM treatments based on the personalized recommendations.

In a further aspect, a system is provided for visualizing wellness treatments. The system comprises a plurality of PBM devices, each capable of collecting wellness data and delivering a wellness treatment; a server, in communication with the PBM devices, configured to collect the wellness data and generate personalized treatment recommendations; and a Virtual Reality (VR) module, in communication with the server, configured to generate a three-dimensional (3D) model of the user's body based on the wellness data, and to display the target areas for the PBM treatment and the potential effects of the treatment.

In still another aspect, a method is provided for visualizing wellness treatments. The method comprises collecting wellness data from a plurality of PBM devices; transmitting the collected wellness data to a server; generating personalized treatment recommendations based on the wellness data; generating a three-dimensional (3D) model of a user's body based on the wellness data using a Virtual Reality (VR) module; displaying the target areas for the PBM treatment on the 3D model; and simulating the potential effects of the treatment on the 3D model.

In another aspect, a method is provided for providing a subscription-based wellness promotion service. The method comprises (a) registering users via a web-based platform and associating each user with at least one photobiomodulation (PBM) device; (b) collecting wellness data from the PBM device of each user; (c) transmitting the collected wellness data to a server; (d) analyzing the wellness data to generate personalized treatment recommendations using at least one artificial intelligence (AI) algorithm; (c) communicating the personalized treatment recommendations to each PBM device; (f) adjusting PBM treatments based on the personalized recommendations; (g) tracking wellness progress over time and adjusting treatment recommendations based on said progress; (h) providing community support features including forums, shared wellness goals, and peer motivation tools; and (i) providing continuous updates to the AI algorithm and to treatment recommendations based on new research and collective user progress data.

In a further aspect, a system is provided for providing a subscription-based wellness promotion service. The system comprises (a) a web-based platform for user registration and interaction; (b) a plurality of photobiomodulation (PBM) devices, each associated with a registered user; (c) a server for receiving wellness data from the PBM devices and providing personalized treatment recommendations; (d) at least one artificial intelligence (AI) algorithm stored on the server for analyzing wellness data and generating treatment recommendations; and (c) a database for tracking user wellness progress and providing continuous updates to the AI algorithm and treatment recommendations.

In another aspect, a wellness system is provided which comprises a plurality of Photobiomodulation (PBM) devices each associated with a user, configured to collect wellness data and communicate said data over a network; a server infrastructure equipped with a hypervisor software for managing multiple virtual machines; a virtual machine running an artificial intelligence engine configured to analyze the wellness data from the PBM devices and generate personalized treatment recommendations; a second virtual machine running a distributed ledger system configured to securely record the wellness data and treatment recommendations; a third virtual machine running a user interface software configured to facilitate interaction between the user and the wellness system; wherein the hypervisor software isolates the artificial intelligence engine, the distributed ledger system, and the user interface in their respective virtual machines and manages resources among these virtual machines.

In a further aspect, a method for managing a wellness system is provided. The method comprises collecting wellness data from a plurality of PBM devices each associated with a user; transmitting the wellness data to a server infrastructure running multiple virtual machines managed by a hypervisor software; analyzing the wellness data with a first virtual machine running an artificial intelligence engine to generate personalized treatment recommendations; securely recording the wellness data and personalized treatment recommendations in a second virtual machine running a distributed ledger system; facilitating interaction between the user and the wellness system via a user interface running on a third virtual machine; isolating the artificial intelligence engine, the distributed ledger system, and the user interface in the first, second and third virtual machines, respectively; and managing resources among the first, second and third virtual machines using the hypervisor software.

In still another aspect, a wellness system is provided which utilizes a containerized software infrastructure. The system comprises a plurality of photobiomodulation (PBM) devices each associated with a user, each device capable of collecting wellness data and providing light therapy based on personalized treatment recommendations; a server equipped with a container orchestration platform for managing multiple software containers that provide discrete, isolated services; at least one software container functioning as a wellness data collector, receiving wellness data from the PBM devices; at least one software container functioning as an artificial intelligence (AI) engine, analyzing collected wellness data to generate personalized treatment recommendations; at least one software container functioning as a communications module, transmitting treatment recommendations to the PBM devices; at least one software container functioning as a distributed ledger system, recording wellness data and treatment recommendations; and at least one software container functioning as a user interface module, providing users with access to their wellness data, treatment recommendations, and other system features.

In yet another aspect, a method is provided for promoting wellness using a containerized software infrastructure. The method comprises collecting wellness data from a plurality of photobiomodulation (PBM) devices associated with individual users; transmitting the collected wellness data to a server equipped with a container orchestration platform; processing the wellness data in a container functioning as a wellness data collector; analyzing the collected wellness data using an artificial intelligence (AI) engine operating in a separate container to generate personalized treatment recommendations; transmitting the personalized treatment recommendations to the PBM devices via a communications module operating in a separate container; recording the wellness data and treatment recommendations in a distributed ledger system operating in a separate container; and providing users with access to their wellness data, treatment recommendations, and other system features via a user interface module operating in a separate container.

In a further aspect, a method for promoting wellness using a plurality of photobiomodulation (PBM) devices is provided. The method comprises installing a software client on each PBM device; collecting wellness data from each PBM device, and transmitting the collected data to a server in a secure manner; running an artificial intelligence (AI) engine in a separate virtual machine to analyze the collected wellness data and generate personalized treatment recommendations; storing the wellness data and the treatment recommendations in a distributed ledger system running in a separate virtual machine; displaying an interactive 3D model of the user's body using a virtual reality (VR) module running in a separate virtual machine, allowing the user to visually identify target areas for PBM treatment, observe potential treatment effects, and manipulate the model to gain a better understanding of their wellness data; providing the personalized treatment recommendations to each PBM device; using a hypervisor to manage the assignment of system resources to the virtual machines and monitor their performance; utilizing a container orchestration platform to automatically adjust the number of container instances based on real-time system load, ensuring efficient resource utilization and performance; and providing a web-based subscription service with tiered pricing plans offering different levels of service features.

In still another aspect, a wellness system operating within a web3 infrastructure is provided. The wellness system comprises a plurality of Photobiomodulation (PBM) devices, each associated with a user and capable of generating and transmitting user-specific wellness data; a server equipped with a smart contract interpreter capable of executing smart contracts within a distributed ledger environment; and a distributed ledger for storing and managing the user-specific wellness data and treatment regimens, wherein the ledger operates within the web3 infrastructure and is accessible by the plurality of PBM devices and the server.

### DETAILED DESCRIPTION

Despite its tantalizing potential, the implementation of PBM faces numerous challenges. For example, the actinic radiation associated with PBM may be applied at various wavelengths, flux and duration. Moreover, the optimal usage parameters for PBM may vary from user to user, depending on various considerations such as the user's health, medical conditions, age, and sex.

The foregoing considerations are further complicated by the fact that some of the effects of PBM are known to be biphasic or multiphasic. For example, mediators of PBM such as adenosine triphosphate (ATP) and mitochondrial membrane potential exhibit biphasic patterns, while others such as mitochondrial reactive oxygen species show a triphasic dose-response. [Huang Y Y, Sharma S K, Carroll J, Hamblin M R. Biphasic dose response in low level light therapy, an update. Dose Response. 2011; 9(4):602-18. doi: 10.2203/dose-response.11-009.Hamblin. Epub 2011 Sep. 2. PMID: 22461763; PMCID: PMC3315174; see also Genoveva Lourdes Flores Luna, Ana Laura Martins de Andrade, Patricia Brassolatti, Paulo Sérgio Bossini, Femanda de Freitas Anibal, Nivaldo Antonio Parizotto, and Angela Merice de Oliveira Leal. Biphasic Dose/Response of Photobiomodulation Therapy on Culture of Human Fibroblasts. Photobiomodulation. Photomedicine, and Laser Surgery.July 2020.413-418.http://doi.org/10.1089/photob.2019.4729] This creates a situation where PBM may be physiologically effective (or increasingly effective) up to a certain dosage but may lose its effectiveness thereafter.

Since dose-response relationships have a direct bearing on the efficacy of PBM, the determination of effective parameters for PBM treatment is of paramount importance. Accordingly, the physiological mechanisms implicated by PBM have been closely studied, and various experiments have been conducted to elucidate the effect and optimal range of various treatment parameters. However, these efforts are complicated by the sheer number of factors at play. As a result, even for more narrowly defined groups such as Alzheimer's patients, PBM parameters, such as the dose-effect relationship for PMB in AD treatment, remain to be explored and optimized. [Zhang, R., Zhou, T., Liu, L., Ohulchanskyy, T. Y., & Qu, J. (2021). Dose-effect relationships for PBM in the treatment of Alzheimer's disease. Journal of Physics D. Applied Physics. 54],

It has now been found that the foregoing infirmities may be addressed with the systems and methodologies disclosed herein. In a preferred embodiment of these systems and methodologies, an artificial intelligence (AI) system is used to collect and/or analyze user data collected from a community of users, and then (possibly after training the AI system on a training data set) modulate treatment parameters to collect statistical data associated with changes to the treatments implemented. This data is then analyzed with, for example, machine learning to discern useful correlations or patterns that may be utilized to inform and optimize recommended treatments for individual users, subgroups of users, or the community as a whole. Among its other advantages, this approach may identify sets of users that benefit from similar PBM parameters.

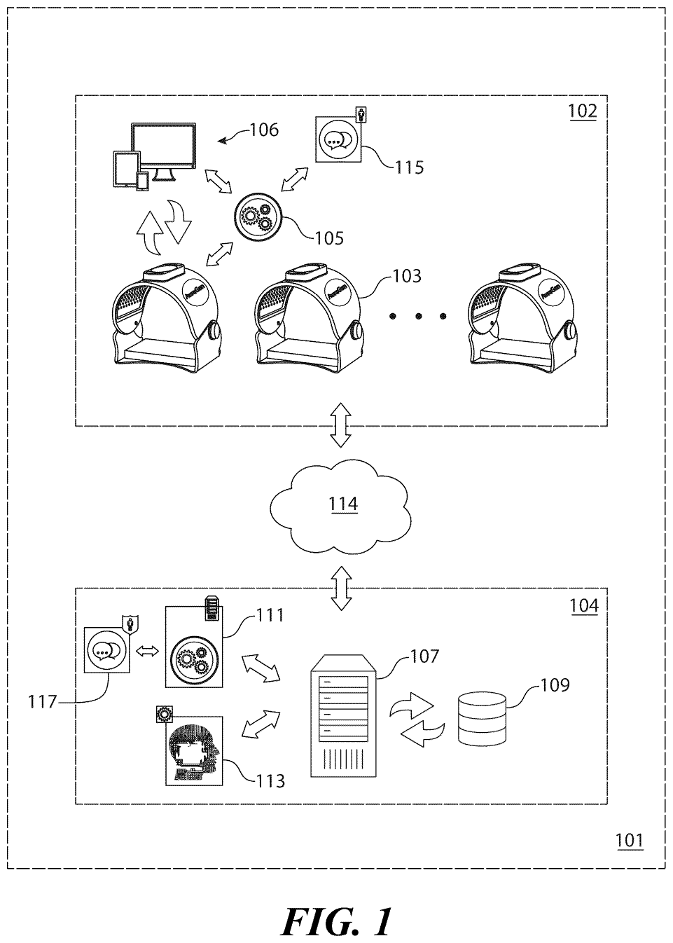

FIG. 1 depicts a particular, nonlimiting embodiment of a system in accordance with the teachings herein for promoting wellness through the use of PBM. The system 101 comprises a front end 102 (or client side) and a backend 104 (or server side) which are in communication with each other over the cloud 114. The front end 102 includes a plurality of client PBM devices 103 and a software client 105, an instance of which is installed on each PBM device 103 or on a user device 106 which is in communication with a PBM device 103. The user devices 106 may be, for example, smart phones, smart TVs, PCs (including tablet, laptop and desktop PCs), or other end user devices equipped with a suitable operating system capable of running or implementing the software client 111 (the device running or implementing the software client 111 is hereinafter referred to as the client device).

The back end 104 includes a server 107 which is equipped with server software 111 and which is in communication with a wellness database 109 and an AI engine 113. The AI engine 113 receives wellness data from the wellness database 109 and treatment objectives from the plurality of client devices 103 via the software client 105 and provides a range of therapy treatment recommendations to include light, sound, guided meditation, and tactile stimulation to the plurality of client devices 103. The therapy treatment recommendations may be based, for example, on the application of machine learning to the wellness database 109, which may be updated with statistical information received from the community of users that had variations in the treatments provided. For example, data (including various types of biometric data) may be obtained from the community of users before, during and after one or more treatment sessions with a light, sound, guided meditation, or tactile stimulation therapy device. Such data may include body sensor readings, biometric data or other data relating to brain wave activity, cytokine profiles, pulse rate, pulse rate variability, blood pressure, rate of respiration, blood glucose levels, measurements of mood and brain cognitive levels using user questionnaires, electronic game data, and the like.

The PBM devices 103 are the physical devices used to administer PBM therapy. These may be handheld devices, wearable devices, or stationary devices, and are equipped with hardware capable of emitting light at the specific wavelengths required for PBM. Preferably, however, the PBM devices 103 are the PBM devices described in U.S. Ser. No. U.S. Ser. No. 17/195,068 (Barron et al.), which is incorporated herein by reference in its entirety. The PBM devices 103 (or the associated user devices 106) are equipped with suitable connectivity to a network 114. Such connectivity may include, for example, one or more Wi-Fi or Bluetooth devices for communicating with the server 107 via a router or other suitable means, and may also be equipped with the necessary hardware (having the appropriate storage and processing capability) to run the software client 105 (it being understood that, in some embodiments, the software client may be run instead on a suitable user device 106 which is in communication with a PBM device).

As previously noted, a software client 105 is installed on each PBM device 103, or on a user device 106 in communication with the PBM device 103. The software client 105 collects data from the user, such as user input data and sensor data from the PBM device 103, and transmits this information to the server 107 via the cloud 114. The PBM device 103 is also adapted to receive treatment recommendations from the server 107 and present them to the user in a user-friendly manner by way of a user interface 115. The software client 105 is adapted to control the operation of the PBM device 103 based on the received recommendations.

The server 107 is preferably a high-performance computer system capable of handling requests from numerous client devices simultaneously. It is preferably equipped with a high-speed, high-capacity network interface for receiving and transmitting data, as well as ample storage for the wellness database. The server's processing power will typically need to be sufficient to run the AI engine effectively.

The wellness database 109 is hosted on the server 107 or a device associated therewith (here, it is to be understood that, in a given implementation, the server 107 may be a plurality of servers). The wellness database 109 may be implemented using a suitable database management system (DBMS) capable of storing and organizing large amounts of data efficiently. The DBMS allows for quick retrieval and updating of data. The wellness database 709 stores all the wellness data received from the users, preferably organized by user ID.

The server software 111 is adapted to handle incoming data from the client devices, and to store this data in the wellness database 109. It is also adapted to receive treatment objectives from the users, and to interface with the AI engine 113 to generate PBM treatment recommendations based on these objectives and the wellness data. The server software 111 then transmits these recommendations back to the client devices.

The AI engine 113 is preferably a software component that uses suitable machine learning algorithms or artificial intelligence to analyze the wellness data and to generate PBM treatment recommendations. The AI engine 113 may be based on one or more types of AI algorithms (the choice of which may depend on the requirements of the system or the problems to be addressed), such as neural networks, deep learning models, or other machine learning methods. The AI engine 113 will typically require access to the server's processing power and storage and is adapted to interface with the server software 111 to receive inputs and to provide outputs.

As previously noted, the system 101 preferably also includes a user interface 115 on the client side 102 for users to input their treatment objectives, view their wellness data and treatment recommendations, and interact with a PBM device 103. Similarly, the server side 104 also preferably includes an administrative interface 117 for monitoring and managing the system, as well as interfaces for data scientists or other professionals to work with the wellness database 109 and the AI engine 113.

The client-side user interface 115 may be a mobile application or a web application that is preferably designed as a responsive and user-friendly solution. This application is preferably adapted to interact with the PBM device 103 via Bluetooth, Wi-Fi, ethernet cable, or by other suitable means, and to allow the user to access and control the functionalities of the PBM device 103 and the system 101 as a whole.

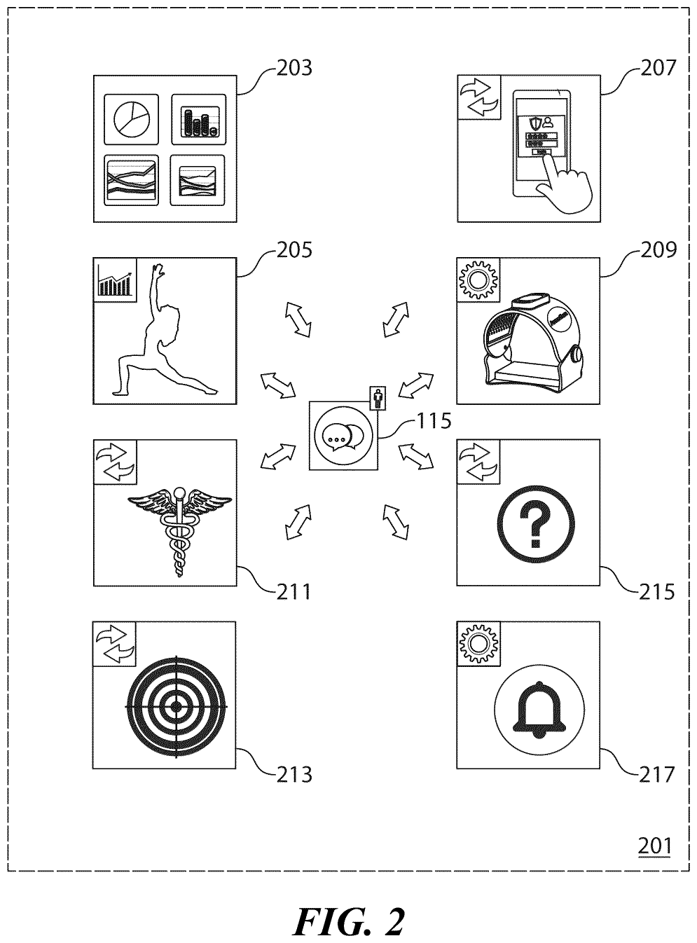

A particular, non-limiting embodiment of the user interface 115 and the functionality associated therewith is depicted in FIG. 2. As seen therein, the user interface 115 includes a user login 207 which is typically activated when the application is launched. The user login 207 preferably prompts the user to enter a unique username and/or password, although in some embodiments it may be equipped to authenticate a user by capturing biometric data (as, for example, through facial recognition, fingerprints, or retinal scans). This process ensures that the data related to each user remains private and secure. In some embodiments, the user login 207 may be adapted to remember certain login details associated with a user for future convenience.

Once logged in, the user is presented with a dashboard 203. This dashboard 203 may display an overview of the user's wellness data such as, for example, recent PBM sessions, current wellness scores or wellness trends over time. It may also display reminders or alerts for upcoming therapy sessions.

The user interface 115 is also preferably equipped with a user goals section 213 which allows users to input and edit their treatment objectives. Here, users can specify various wellness goals such as, for example, pain management, mood improvement, sleep regulation, and the like. User input may be captured through the provision of suitable dropdown options, checkboxes, sliders or text input, depending on the type of data required.

The user interface 115 also preferably includes a wellness data module 205. This module allows users to view a detailed report of their wellness data collected over time. This data may be displayed in the form of graphs, charts, or tables to help users understand their progress and wellness trends. The wellness data module 205 will also typically give the user the option to view data for specific periods and to generate corresponding (e.g., weekly, monthly, or yearly) reports.

The user interface 115 also preferably includes a module dedicated to treatment recommendations 211 which allows users to receive PBM treatment recommendations 211 generated by the AI engine 113 (see FIG. 1). The treatment recommendations 211 may include details such as the recommended light wavelength (or wavelengths), intensity, duration of exposure, and the frequency of sessions. In some embodiments, the application may provide a simple “Start Session” button, which directly communicates with the PBM device 103 (see FIG. 1) to begin a therapy session according to these recommendations.

The user interface 115 also preferably includes a device control 209 which provides features to manually control the PBM device. Such features may relate to turning the PBM device on or off, adjusting the light intensity of the PBM device, setting a timer for a PBM session, selecting the light wavelength(s) utilized, or taking other actions which may depend on the capabilities of the device.

The user interface 115 also preferably includes a notifications module 217 which allows the application to send push notifications to remind users of upcoming sessions, inform them of new treatment recommendations, or alert them of any significant changes in their wellness data.

The user interface 115 also preferably includes a user support module 215. This module allows users to access FAQs, troubleshooting guides, or contact details for technical support.

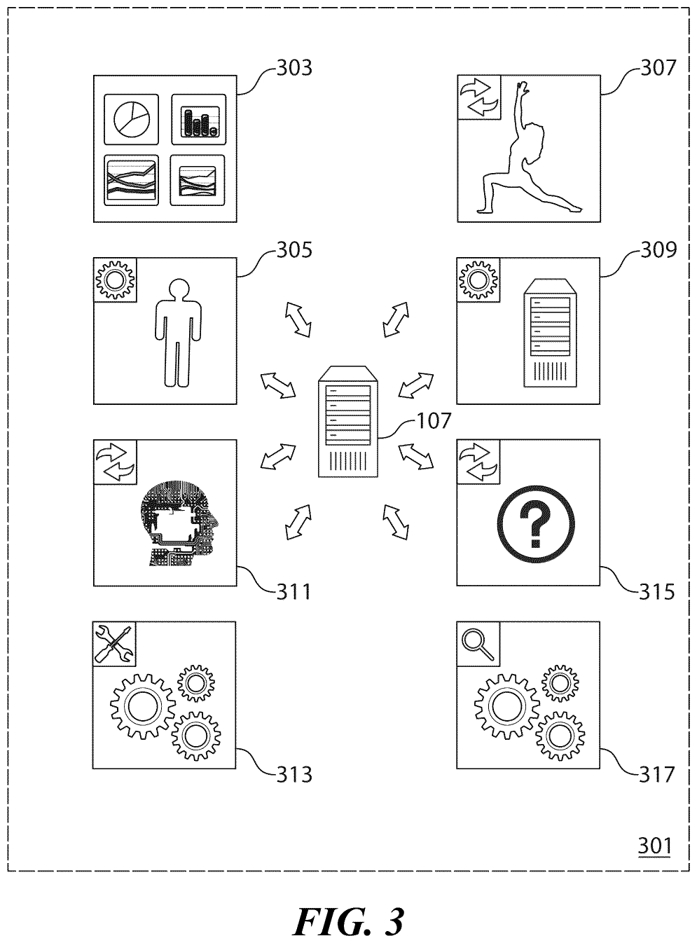

A particular, non-limiting embodiment of the server-side administrative interface 117 and the functionality associated therewith is depicted in FIG. 3. As seen therein, the server-side administrative interface 117 is preferably a web application with different access levels for administrators, data scientists, and other professionals. It preferably provides features for system monitoring, user management, data analysis, and AI engine management.

The server-side administrative interface 117 is preferably equipped with a dashboard 303 that provides an overview of the system. The dashboard 303 may show, for example, real-time statistics and performance metrics, such as total number of active users, number of PBM devices connected, recent activity logs, AI engine performance metrics, and any system alerts or notifications.

The server-side administrative interface 117 is also preferably equipped with a user management module 305 that allows administrators to manage user accounts. Thus, the user management module 305 preferably allows administrators to add, remove, or modify user accounts, reset passwords, and view user activity logs. It may also be equipped with features for grouping users, managing user roles and permissions, and tracking user status.

The server-side administrative interface 117 is also preferably equipped with a device management module 309. The device management module 309 preferably allows administrators to view and manage PBM devices connected to the system. This may include the ability to view device status (online/offline), device usage statistics, or device errors or alerts. The device management module 309 may also provide administrators with the ability to remotely troubleshoot devices or push software updates.

The server-side administrative interface 117 is also preferably equipped with a wellness database interface 307. The wellness database interface 307 preferably provides a means for data scientists or other professionals to access and analyze the wellness data. This interface preferably provides a suite of data visualization and analysis tools, including query builders, data filters, chart generators, and statistical analysis functions. The wellness database interface 307 also preferably includes functionality to allow an administrator to export data for further analysis in other software.

The server-side administrative interface 117 is also preferably equipped with an AI engine interface 311. The AI engine interface 311 preferably allows professionals to work with the AI engine. For example, the AI engine interface 311 may provide a means by which such parties can train and validate AI models, adjust AI parameters, test AI performance, and view AI learning logs. It may also provide the ability to upload and integrate new AI models.

The server-side administrative interface 117 is preferably further equipped with a system monitoring module 317. This module preferably provides tools for monitoring the performance and health of the system. The server-side administrative interface 117 may include network performance monitoring, server resource usage monitoring, error log viewers, and system event logs.

The server-side administrative interface 117 is preferably further equipped with a system settings module 313. The system settings module 313 preferably provides a means by which administrators may adjust system settings such as network settings, security settings, and system configurations. The system settings module 313 also preferably allows administrators to manage system backups and perform system updates.

The server-side administrative interface 117 is also preferably equipped with a support & help desk module 315. This module preferably facilitates communication between system users and administrators. It may also allow users to raise support tickets, which administrators can view and respond to.

In a typical application of the system depicted in FIGS. 1-3, the software client 105 is installed on each PBM device 103 (or on a user device 106 which is in communication with the PBM device 103). The software client 105 serves as the communication interface between the PBM device 103 and the server 107, enabling the exchange of wellness data and treatment objectives. It may also control the operations of the PBM device 103 based on instructions received from the server 107.

Wellness data from each user (which may include, for example, information such as user's medical history, current health condition, lifestyle habits, and individual preferences) is collected and forms a comprehensive wellness database. This data may be collected via user inputs or through additional sensors integrated into the PBM devices 103 that can measure relevant health parameters such as heart rate, body temperature, blood oxygen levels, and the like.

The collected wellness data, alongside the treatment objectives defined by each user, are transmitted to the server 107 via the installed software client 105. These treatment objectives may be diverse, ranging from alleviating specific symptoms, promoting overall wellness, accelerating recovery from injuries, to improving skin health, among others.

Once received at the server 107, the wellness data and treatment objectives are processed by the AI engine 113. The AI engine 113 employs advanced data analysis and machine learning techniques to understand the unique wellness needs and goals of each user. Based on this understanding, the AI engine generates personalized PBM treatment recommendations for each user. These recommendations may include details such as, for example, the duration of light therapy, the frequency of therapy sessions, the intensity of light to be used, the wavelengths of light to be utilized, the specific areas of the body to be targeted, the frequency (or frequencies) of oscillation (e.g., flicker) in the light, and any accompanying therapy options (such as, for example, binaural beats, the application of tactile stimulation, or the like).

These AI-generated recommendations are then transmitted back to the respective PBM devices 103. The user can review these recommendations, and if accepted, the software client 105 on the PBM device 103 will adjust the PBM treatment parameters accordingly. By this process, the method ensures that each user receives a personalized PBM treatment plan that is tailored to their specific needs and treatment objectives, ultimately promoting wellness effectively and efficiently.

The systems and methodologies disclosed herein may leverage various systems, algorithms or features of artificial intelligence, machine learning algorithms, and neural networks.

Artificial intelligence systems suitable for use in the systems and methodologies disclosed herein may include items spanning the spectrum from rule-based systems to complex machine learning models. In these systems and methodologies, AI may be utilized to process data, make decisions, and provide personalized treatment recommendations.

Various machine learning systems and algorithms may be utilized in the systems and methodologies disclosed herein. Examples of machine learning algorithms which may be utilized in the systems and methodologies described herein include supervised learning algorithms, unsupervised learning algorithms, and reinforcement learning algorithms.

Supervised learning algorithms may be utilized, for example, if labeled training data is available. Such labeled training data may be, for example, previous wellness data and associated successful treatments. Algorithms such as support vector machines, linear regression, or random forests may be utilized to predict the most effective treatments based on new wellness data. Supervised learning algorithms may play an essential role in multiple areas in the wellness systems described herein, particularly in generating personalized photobiomodulation (PBM) recommendations based on the wellness data collected from users. In general, supervised learning algorithms may help the wellness system to personalize PBM treatment recommendations, adapt to changes in users' wellness data, and improve the effectiveness of PBM treatments over time.

For example, supervised learning algorithms may be utilized in model training in the systems and methodologies disclosed herein. The AI engine in the server, with the use of supervised learning algorithms, may be trained on a large dataset of wellness data and corresponding PBM treatment outcomes. The goal of such training may be to learn a function that maps input data (i.e., wellness data) to the desired output (i.e., successful PBM treatment outcomes).

Supervised learning algorithms may also be utilized in making predictions in the systems and methodologies disclosed herein. In some embodiments, after the training process, the supervised learning model may be able to predict the optimal PBM parameters for a new user or a user with updated wellness data. The model may accept the wellness data of a user as input and output the recommended PBM parameters that are likely to bring about the best wellness outcomes.

Supervised learning algorithms may also be utilized for continuous learning purposes in the systems and methodologies disclosed herein. In some embodiments, the wellness system may continue to collect wellness data and treatment outcomes from users. This ongoing data may be used to continuously retrain and refine the supervised learning model, improving the accuracy and effectiveness of the PBM recommendations over time.

In some embodiments, the system may use a type of supervised learning called regression to predict continuous outcomes, such as the optimal intensity or duration of PBM treatment. Alternatively, it may use classification to predict categorical outcomes, such as whether a particular type of PBM treatment will be effective or not.

As previously noted, supervised learning in the systems and methodologies disclosed herein may implement various algorithms such as support vector machines (SVMs), linear regression, or random forests.

SVMs are a set of supervised learning methods used for classification and regression. In the systems and methodologies described herein, given the wellness data of users, an SVM may be utilized, for example, to classify users into different categories based on their likely responses to different PBM treatments. The SVM works by mapping the input data to a high-dimensional feature space where a hyperplane may be utilized to separate the different classes or predict a continuous value. This algorithm may be especially useful in situations where the wellness data is not linearly separable or has a high number of dimensions.

Some embodiments of the systems and methodologies described herein may utilize linear regression. Linear regression is a statistical technique that models the relationship between a dependent variable and one or more independent variables. In an illustrative embodiment, for example, linear regression may be utilized in which the dependent variable may be the effectiveness of a specific PBM treatment, and the independent variables may be the wellness data collected from the users. The model would try to fit a linear equation to the observed data. The coefficients in the linear equation could then be used to predict the effectiveness of the treatment for new users.

Some embodiments of the systems and methodologies described herein may also utilize a random forests algorithm. The random forests algorithm is a machine learning algorithm that operates by constructing a multitude of decision trees at training time and outputting the class that is the mode of the classes (classification) or mean prediction (regression) of the individual trees. In an illustrative embodiment, given a new user's wellness data, a random forest algorithm may be used to predict the most effective PBM treatment by passing the data through each decision tree in the forest and combining the results. Use of this algorithm may be especially effective at handling large datasets with many features, and can model complex non-linear relationships.

Various other algorithms may also be utilized in the systems and methodologies described herein, especially in predicting the most effective treatments based on new wellness data. Thus, in addition to SVMs, linear regression, and random forests, various embodiments of the systems and methodologies disclosed herein may utilize neural networks/deep learning, gradient boosting, k-nearest neighbors (K-NN), logistic regression, and native Bayes.

Neural networks/deep learning are models that can identify complex patterns in large datasets by mimicking the human brain's own process of learning. Neural networks may be especially effective in embodiments of the systems and methodologies disclosed herein in which the wellness data includes unstructured data like images or text.

Gradient boosting is a machine learning technique for regression and classification problems, which produces a prediction model in the form of an ensemble of weak prediction models, typically decision trees. It builds the model in a stage-wise fashion and it generalizes them by allowing optimization of an arbitrary differentiable loss function.

K-NN is an algorithm which may be utilized to classify new users based on the similarity of their wellness data to that of existing users. Here, it is to be noted that the “k” in K-NN refers to the number of nearest neighbors the algorithm considers when deciding how to classify the new data.

Logistic regression is an algorithm which may be used to model the probability of a certain class or event existing such as pass/fail, win/lose, alive/dead or healthy/sick. In the case of wellness data, it may be used to predict binary or multi-class outcomes based on the given set of independent variables.

Naive Bayes is a classification technique based on Bayes' theorem with an assumption of independence among predictors. In simple terms, a Naive Bayes classifier assumes that the presence of a particular feature in a class is unrelated to the presence of any other feature. It may be used in the systems and methodologies disclosed herein to predict the likelihood of different treatment outcomes based on the wellness data.

As a particular, non-limiting example of the employment of a Naive Bayes algorithm in the systems and methodologies described herein, a Naive Bayes algorithm may be utilized to predict the most effective photobiomodulation (PBM) treatments based on wellness data. This would generally involve the steps of collecting data, training the model, predicting outcomes, and updating the model.

In the data collection step, the system collects wellness data from the users. This data may include, for example, demographic information, health metrics, lifestyle data, and specific health goals. Additionally, data on user responses to different PBM treatments may be collected, including how effective each treatment was in helping the user reach their health goals.

The collected data is then utilized to train a Naive Bayes classifier. The model is trained to predict treatment success based on the wellness data. In this case, treatment success is the class to be predicted, and the wellness data points are the features. For instance, if three treatments (T₁, T₂, T₃) are available, the model calculates the probability of success for each treatment given the user's wellness data. The ‘naive’ assumption of this algorithm is that each data point (or feature) in the wellness data is independent of all others.

The foregoing information may then be used to predict outcomes. In particular, when a new user inputs their wellness data into the system, the Naive Bayes classifier may be used to predict which PBM treatment is likely to be most successful. The classifier does this by calculating the conditional probability of success for each treatment given the new user's wellness data, and then recommending the treatment with the highest probability of success.

The model is then updated. In particular, as more users use the system and provide feedback on their treatment outcomes, the Naive Bayes classifier may be retrained with this new data. This allows the model to continuously improve and refine its predictions over time.

One skilled in the art will appreciate that the ‘naive’ assumption of the Naive Bayes classifier—that each feature is independent of all others—is often not entirely accurate in real-world data. However, Naive Bayes classifiers can still often make good predictions even when this assumption is violated, especially when the goal is to rank predictions in order of likelihood.

As a particular, non-limiting example of the employment of a gradient boosting algorithm in the systems and methodologies described herein, such an algorithm may be utilized to predict the most effective PBM treatments based on wellness data. This would generally involve the steps of collecting data, preprocessing the data, training the gradient boosting model, predicting the most effective treatment, and continued learning.

In the data collection step, the system collects wellness data from the users, which may include a range of parameters such as, for example, age, lifestyle habits, health conditions, and responses to different PBM treatments. The collected data is then preferably preprocessed to prepare it for ingestion by the learning algorithm. This may involve, for example, normalizing numerical data, addressing missing values, or transforming categorical variables into a suitable numerical form.

The gradient boosting model may then be trained on the preprocessed data. This model is essentially an ensemble of weak prediction models (usually decision trees), and it operates by iteratively adding new models that aim to correct the errors made by the existing ensemble. The idea is to improve the model's predictions gradually (hence the term “gradient”), with each new model pushing a little bit closer to the true output values. In the systems and methodologies disclosed herein, the gradient boosting model may be trained, for example, to predict the success of a particular PBM treatment based on the input wellness data.

The trained model may then be utilized to predict the most effective treatment. Thus, when a new user inputs their wellness data into the system, the trained gradient boosting model may be utilized to predict the likely effectiveness of different PBM treatments for that user. The treatment with the highest predicted effectiveness may then be recommended to the user.

As users interact with the system and provide feedback on the success of their treatments, this new data may be fed back into the gradient boosting model, allowing it to continually learn and improve its predictions over time.

The use of gradient boosting models in the systems and methodologies disclosed herein offers several possible advantages. For example, the use of such models may provide a means by which the performance of the systems and methodologies disclosed herein may improve as more data becomes available. Gradient boosting models can handle a variety of data types, are well adapted to deal with missing data, and are resistant to overfitting, making them a strong choice for systems and methodologies of the type disclosed herein that involve complex, real-world data. Of course, it will be appreciated that gradient boosting models may be computationally intensive to train (especially on large datasets), a factor which should be considered in the overall system design.

As a particular, non-limiting example of the employment of a K-NN algorithm in the systems and methodologies described herein, such an algorithm may be utilized to predict the most effective PBM treatments based on wellness data. This would generally involve the steps of collecting data, preprocessing the data, setting up the K-NN model, predicting the most effective treatment, continuous model updating, and optionally choosing or adjusting a K-value.

In the data collection step, the system collects wellness data from the users, which may include a range of parameters such as, for example, age, lifestyle habits, health conditions, and responses to different PBM treatments. The collected data is preferably preprocessed to make it suitable for the K-NN algorithm. This typically involves normalizing or standardizing the numerical data to ensure that all features are on the same scale. If the data includes categorical variables, these may also need to be converted to a suitable numerical form.

The K-NN model is set up using the preprocessed data. The K-NN algorithm operates by finding the ‘K’ examples in the training data that are closest (most similar) to a given input example, and then predicting the output for the input example based on the outputs for these ‘K’ closest examples.

When a new user inputs their wellness data into the system, the K-NN algorithm may be utilized to predict the likely success of different PBM treatments for that user. This may be accomplished by finding the ‘K’ users in the system's database that have the most similar wellness data to the new user, and then recommending the PBM treatment that was most successful for these similar users. As more users input their wellness data and provide feedback on the success of their treatments, this new data may be added to the system's database, allowing the K-NN predictions to be based on more and more data over time.

The choice of ‘K’ (the number of nearest neighbors to consider) is a key parameter in the K-NN algorithm, and it may greatly affect the algorithm's performance. A smaller ‘K’ can make the algorithm more sensitive to noise and outliers in the data, while a larger ‘K’ can make the algorithm more resistant to noise, but potentially less sensitive to important local patterns. It will thus be appreciated that, in implementing embodiments of the systems and methodologies disclosed herein which utilize a K-NN algorithm, it may be necessary to experiment with different values of ‘K’ to find the best balance for the specific data and task at hand.

The K-NN algorithm is especially desirable for implementations of the systems and methodologies disclosed herein in which there are clear ‘neighborhoods’ of similar examples in the data. The K-NN algorithm is also suited to handle a wide variety of data types. However, the algorithm may be computationally intensive for large datasets, since it requires comparing each new input example to all examples in the training data. Therefore, optimizations or approximate methods may be required in dealing with very large wellness databases.

As a particular, non-limiting example of the employment of logistic regression in the systems and methodologies described herein, such an algorithm may be utilized to predict the likelihood of certain outcomes, such as the effectiveness of a particular PBM treatment. This will generally involve the steps of collecting data, preprocessing the data, setting up the logistic regression model, training the model, using the model to make predictions, continuous model updating, and model interpretation.

In the data collection step, the wellness system gathers data from multiple users. This data may include, for example, information about the user's age, lifestyle habits, specific health conditions, and the effectiveness of various PBM treatments. The collected data may then be preprocessed to prepare it for ingestion by the logistic regression model. Preprocessing may involve, for example, normalizing numerical features, encoding categorical variables, and handling missing data.

The logistic regression model is then established with the preprocessed data. This my involve specifying a binary outcome variable (for example, whether a particular PBM treatment was effective or not) and a set of predictor variables (for example, age, lifestyle habits, or health conditions). The logistic regression model is then trained on this data. This typically involves using an optimization algorithm to find the model parameters that best fit the observed data, in terms of maximizing the likelihood of the observed outcomes given the predictor variables.

Once the model is trained, it may be used to predict the likely success of a given PBM treatment for a new user. In a given implementation, the user may input their wellness data into the system (or have the information input by a third party), and the logistic regression model calculates the predicted probability of the treatment being effective for this user. As more users provide feedback on the effectiveness of their treatments, this new data can be used to continuously update and improve the logistic regression model.

The use of a regression model in the systems and methodologies disclosed herein has several notable advantages. One especially notable advantage is that logistic regression provides interpretable model parameters. For example, each predictor variable is associated with a weight, which represents the change in the log-odds of the outcome variable for a one-unit change in the predictor variable. This may help to elucidate which factors are most important in predicting treatment effectiveness. Of course, it will be appreciated that logistic regression makes certain assumptions, such as the linearity of the log-odds of the outcome variable and the absence of multicollinearity among the predictor variables. Therefore, appropriate checks and data preprocessing steps may be required to ensure these assumptions are met.

As previously noted, various unsupervised learning algorithms may also be used in the systems and methodologies disclosed herein. Unsupervised learning algorithms may be utilized, for example, if labeled training data is unavailable. In such instances, unsupervised learning techniques such as k-means clustering or hierarchical clustering may be employed to identify patterns or groupings in the wellness data.

For example, k-means clustering may be utilized to identify clusters of users with similar wellness data and treatment responses. For instance, the wellness data (including variables such as, for example, age, lifestyle habits, specific health conditions, and responses to various PBM treatments) may be input into a k-means algorithm. The algorithm then partitions the data into ‘k’ clusters, where ‘k’ is a predetermined number, based on the similarity of the data points. For example, the k-means algorithm might identify a cluster of users who are relatively young, live an active lifestyle, and have responded positively to a specific PBM treatment. This information may then be utilized to recommend this treatment to new users with similar characteristics.

The choice of ‘k’ in the k-means clustering may be determined using suitable methods. These include, without limitation, the elbow method, silhouette analysis, through cross-validation, or by use of the gap statistic, the Davies-Bouldin Index (DBI), or the Calinski-Harabasz Index.

The Elbow method is a technique for determining the optimal number of clusters (k) in k-means clustering. The method is based on the concept of within-cluster sum of squares (WCSS), which is the sum of the squared distances between each data point in a cluster and the centroid of that cluster. As the number of clusters increases, the WCSS decreases because the data points will be closer to their respective centroids. However, after a certain point, the decrease in WCSS becomes less pronounced as more clusters are added. This point, where the reduction in WCSS begins to level off, is called the “elbow”. The number of clusters corresponding to this elbow point is typically considered the optimal number of clusters.

As a particular, non-limiting embodiment of the use of the Elbow method in determining the choice of ‘k’ in the k-means clustering algorithm, k-means clustering is performed on the data for a range of k values (e.g., from 1 to 10). For each k, the WCSS is calculated. The WCSS values are then plotted against the corresponding k values, and the plot is analyzed to determine the k value for which the reduction in WCSS begins to slow down (i.e., the elbow point). The “elbow” in the plot represents the point where adding more clusters does not significantly improve the within-cluster variance (WCSS), which may be considered a good balance between maximizing the number of clusters (k) and minimizing WCSS. The Elbow method may be somewhat subjective as it requires a visual inspection of the plot and the “elbow” may not always be clear or well-defined.

Silhouette analysis is another method that can be utilized to determine the optimal number of clusters ‘k’ in a dataset for k-means clustering in the systems and methodologies described herein. The silhouette value is a measure of how similar an object is to its own cluster compared to other clusters. The silhouette value ranges from −1 to +1, where a high value indicates that the object is well matched to its own cluster and poorly matched to neighboring clusters.

As a particular, non-limiting embodiment of the use of silhouette analysis in determining the choice of ‘k’ in the k-means clustering algorithm, k-means clustering is performed on the data for a range of k values. For each k, the silhouette score is computed. This score is the mean silhouette coefficient over all the instances. Many programming languages have built-in functions for computing silhouette scores. For example, in Python's scikit-learn, the function metrics.silhouette_score( ) may be utilized. The silhouette scores are then plotted against the corresponding k values. The optimal number of clusters k is the one that maximizes the silhouette score.

In more detail, the silhouette score for each instance involves two distances: (1) the mean distance between the instance and all other instances in the same cluster, which is the average intra-cluster distance (a); and (2) The mean distance between the instance and all instances in the next closest cluster, which is the average nearest-cluster distance (b).

The silhouette coefficient(s) for an instance is given by the formula:

$$s = ( b - a ) / max ( a , b ) \qquad [ EQUATION 1 ]$$

This score indicates how close each instance in one cluster is to the instances in the neighboring clusters. The silhouette score is computed on every instance, and the average score indicates how well each instance lies within its cluster. A value near +1 indicates that the instance is far away from the neighboring clusters. A value of 0 indicates that the instance is on or very close to the decision boundary between two neighboring clusters. Negative values indicate that those instances might have been assigned to the wrong cluster.

Unlike k-means, hierarchical clustering does not require the number of clusters to be predefined. Instead, it constructs a hierarchy of clusters, which may be visualized using a dendrogram. In embodiments of the systems and methodologies described herein, hierarchical clustering may be used to identify hierarchies or nested groupings within the wellness data. For example, at a high level, the data may be split into two clusters based on age (e.g., below 50 and above 50). Each of these clusters may then be further divided based on lifestyle habits, and so on. This kind of hierarchical insight may help provide more personalized PBM treatment recommendations.

In both methods, the choice of similarity measure (e.g., Euclidean distance, cosine similarity, etc.) and the handling of different types of variables (e.g., binary, categorical, numerical) will typically be important considerations. The results of these unsupervised techniques may be used to provide personalized treatment recommendations, as well as for exploratory data analysis to identify trends and patterns in the wellness data.

As previously noted, the Gap statistic, the Davies-Bouldin Index (DBI), the Calinski-Harabasz Index (CHI), and cross-validation may also be used to determine the optimal number of clusters ‘k’ in a dataset for k-means clustering in the systems and methodologies described herein. Each of these methods has advantages and disadvantages, and the optimal choice of method will typically depend, for example, on the type of data distribution. It will also be appreciated that combinations of the foregoing methods may be utilized to ensure a more robust estimation of the optimal number of clusters.

The Gap statistic compares the total intracluster variation for different values of k with their expected values under null reference distribution of the data (that is, a distribution with no apparent clustering). The optimal number of clusters k is usually where the gap statistic reaches its maximum.

The DBI is the average similarity measure of each cluster with its most similar one, where similarity is the ratio of within-cluster distances to between-cluster distances. Thus, clusters which are farther apart and less dispersed will result in a better score. The optimal number of clusters k is the one that minimizes the DBI.

The CHI (also known as the Variance Ratio Criterion) is the ratio of the sum of between-clusters dispersion and of inter-cluster dispersion for all clusters (where dispersion is defined as the sum of distances squared). The optimal number of clusters k is the one that maximizes the CHI Index.

Cross-validation is generally used in supervised learning to ensure that the model is not overfitting the data. However, it may also be used for determining the number of clusters by clustering the data for various values of k and then applying a cross-validation evaluation (such as, for example, the Rand Index) to determine the quality of the clustering. The optimal number of clusters k would then be the one that maximizes the cross-validation score.

Various reinforcement learning algorithms may be utilized in the systems and methodologies disclosed herein. These algorithms may be utilized, for example, to continually refine the treatment recommendations as new data is received. In such embodiments, the AI learns over time which recommendations lead to the best outcomes, continually updating its approach.

Reinforcement learning (RL) is a type of machine learning that involves an agent learning to make decisions by taking actions in an environment to achieve a goal. The agent receives rewards or penalties (positive or negative rewards) as feedback for its actions and learns to make better decisions over time by maximizing the total reward. Reinforcement learning may be employed in the systems and methodologies described herein in various ways.

For example, RL may be utilized to make personalized recommendations to a user. In such applications, the AI system may act as an agent that recommends specific PBM treatments to users based on their wellness data. The agent may be rewarded when users report positive outcomes (such as, for example, improved wellness scores, achievement of personal health goals, or high user satisfaction scores) and may be penalized for negative outcomes. Over time, the agent learns to recommend treatments that are more likely to result in positive outcomes for each individual user, effectively personalizing the wellness regimen.

RL may also be used for treatment optimization in the systems and methodologies disclosed herein. In such applications, RL may be utilized to optimize the treatment plans generated by the AI. For instance, if the treatment plan involves different light therapies at varying intensities and durations, RL may be utilized to identify the best combination of these variables to optimize wellness outcomes. The system would experiment with different combinations, receive feedback based on user-reported outcomes or biometric data, and learn to fine-tune the treatments accordingly.

RL may also be used for interactive engagement in the systems and methodologies disclosed herein. In such applications, RL may be used to improve the user interface and user experience of the system. The AI system may learn from user interactions and responses to guide the design of the user interface, suggest more relevant wellness content, or tailor the system's communication style to increase user engagement and satisfaction. Positive user interactions (for example, frequent use, high engagement rates, or positive feedback) would be rewarded, and the system would learn to repeat and optimize actions leading to these outcomes.

It will be appreciated that the use of reinforcement learning requires careful design, as inappropriate reward structures may lead to unintended or undesirable consequences. For example, if the system is rewarded only for user engagement, it might favor more frequent, potentially annoying interactions over less frequent but more meaningful interactions. Similarly, a system that is rewarded for immediate wellness outcomes may neglect long-term user wellbeing. It is thus desirable to design the reward structure to align with the overall goals of the wellness system.

Various neural networks may be utilized in embodiments of the systems and methodologies disclosed herein. These include, without limitation, Feedforward Neural Networks (FNN), Convolutional Neural Networks (CNN), recurrent Neural Networks (RNN), Long Short-Term Memory (LSTM), Gated Recurrent Units (GRU), Radial Basis Function Neural Networks (RBFNN), Deep Belief Networks (DBN), Autoencoders (AE), Generative Adversarial Networks (GAN), and Self-Organizing Maps (SOM). The choice and usage of each neural network may depend, for example, on the specifics of the data, the problem at hand, and the desired outcome. A careful selection and combination of these methods, along with robust data collection and preprocessing, may be important in the implementation of successful AI-driven solutions in the systems and methodologies disclosed herein.

FNNs are the simplest form of neural networks, where information moves in only one direction—forward—from the input nodes, through the hidden nodes (if any), and to the output nodes. There are no loops in the network. FNNs may be utilized in the systems and methodologies disclosed herein, for example, to simplify prediction tasks, such as estimating wellness outcomes based on a user's previous data and treatment objectives.

For example, the system may collect relevant wellness data and treatment objectives from the user. This data may include physiological parameters (such as, for example, heart rate, skin temperature, etc.), lifestyle factors (such as, for example, diet, exercise, stress levels), genetic data, treatment objectives (such as, for example, reducing pain, improving mood, healing a skin condition), and past responses to PBM treatments.

The collected data would typically need to be preprocessed before it could be used to train the FNN. Preprocessing steps might include normalizing the data, handling missing values, and encoding categorical variables.

Next, the FNN would be trained on this preprocessed data. The input layer of the FNN will typically include nodes for each of the data features, and the output layer will typically contain nodes for each of the predicted outcomes (e.g., wellness indicators after treatment). The network may also include one or more hidden layers, which may help the model learn complex relationships between the inputs and outputs. The weights between the nodes may be adjusted during training to minimize the difference between the network's predictions and the actual outcomes.

Once the FNN is trained, it may be used to predict wellness outcomes based on new data. After a user's data and treatment objectives are input into the network, it outputs estimated wellness outcomes. These predictions may then inform the PBM treatment recommendations provided to the user. The FNN may be periodically retrained or fine-tuned as new data is collected to ensure that the model's predictions remain accurate as more information is gathered or if user's wellness states or treatment objectives change over time.

It will be appreciated that the foregoing is a high-level overview of how an FNN may be utilized in the systems and methodologies disclosed herein. In various embodiments of these systems and methodologies, the actual implementation may require careful selection of the network architecture (for example, the number of layers and nodes in the network), the learning algorithm (such as, for example. gradient descent or backpropagation), and the loss function (such as, for example, mean squared error for regression tasks). In some embodiments, the model may be validated using techniques such as cross-validation to ensure that it generalizes well to new data.

CNNs are primarily used for image processing, pattern recognition, and video analysis. CNNs are a type of deep learning model and are designed to automatically and adaptively learn spatial hierarchies of features from tasks such as object detection or face recognition. CNNs may be utilized in the systems and methodologies disclosed herein, for example, to identify changes in users as they undergo PBM treatment. For example, if the system includes image data (such as skin condition photos in dermatological treatments), a CNN may be used to analyze these images and potentially identify conditions or changes that might influence treatment recommendations.

As a specific, nonlimiting example of an embodiment of a system or methodology of the type described herein which utilizes a CNN, suppose the system is gathering skin images from users over time, perhaps to monitor the progress of a skin condition under PBM treatment or to identify any new skin-related issues. The collected images will typically need to be preprocessed before they can be fed into the CNN. This may include, for example, resizing the images to a consistent size, normalizing the pixel values, or augmenting the dataset with rotated, shifted, or otherwise modified images to improve the robustness of the model.

A CNN generally consists of convolutional layers, pooling layers, and fully connected layers. Convolutional layers apply a series of filters to the input, which allow the network to automatically and adaptively learn spatial hierarchies of features. Pooling layers reduce the spatial size of the representation, reducing the amount of parameters and computation in the network. Fully connected layers perform classification on the features extracted by the convolutional layers and down-sampled by the pooling layers.

The CNN may be trained on the preprocessed images. The images would be input into the network, and the network's weights would be adjusted to minimize the difference between its predictions and the actual labels (i.e., the known skin conditions associated with each image). Once trained, the CNN may be utilized to analyze new skin images. When a new image is input into the network, it will output a prediction for the skin condition in the image based on the learned features. This may include identifying conditions that are present, monitoring the progress of existing conditions, or even predicting future changes based on current trends.

The CNN's analysis may then be utilized to inform the PBM treatment recommendations. For example, if the CNN identifies a new skin issue, the treatment recommendations may be adjusted to address it. Similarly, if the CNN determines that a user's skin condition is improving, the treatment may be continued or adjusted to maintain the progress.

The CNN will preferably be retrained or fine-tuned periodically as new image data is collected to ensure that it remains effective at identifying and monitoring skin conditions. One skilled in the art will appreciate that, although CNNs are a powerful tool for image analysis, they may require a large amount of labeled data for training, and their output requires careful interpretation.

RNNs (unlike FNNs) have internal loops, allowing them to process sequences of data, making them well-suited for time-series analysis. They have “memory” in that the output for each element in the sequence is dependent on the computations for the preceding elements. RNNs may be utilized in the systems and methodologies disclosed herein, for example, to process sequential wellness data from users such as, for example, changes in vital signs or symptom ratings over time. The RNN could identify patterns in these sequences to help inform treatment recommendations.

As a specific, nonlimiting example of an embodiment of a system of the type described herein which utilizes an RNN, the system may collect sequential wellness data from users over time. This may include time-series data such as, for example, changes in vital signs (heart rate, blood pressure, etc.) or symptom ratings. The sequence in which these data points were collected would be an important part of the data.

Before the sequential data can be input into the RNN, it may need to be preprocessed. This may involve normalizing the data, handling missing values, and formatting the data into appropriate sequences. For example, if the RNN is a Long Short-Term Memory (LSTM) network, it may be necessary to format the data into sequences of a certain length (e.g., representing a week's worth of data) that overlap with each other. The RNN may then be trained on these sequences. The network would learn to update its internal state based on each new data point in the sequence, allowing it to capture patterns over time. For example, it might learn that a certain pattern of changes in vital signs often precedes a flare-up of a user's symptoms.

Once trained, the RNN may be used to make predictions about future wellness outcomes based on new sequences of data. For instance, it may predict whether a user's symptoms are likely to worsen in the near future based on their most recent vital signs. These predictions may be used to inform PBM treatment recommendations. For example, if the RNN predicts a high risk of symptom flare-up, the system might recommend a preventive PBM treatment. Conversely, if the RNN predicts stable or improving symptoms, the current treatment regimen may be continued. As with other types of machine learning models, it may be necessary to retrain or fine-tune the RNN periodically as new data is collected to ensure its predictions remain accurate.

It will be appreciated that the training of RNNs may be complicated by issues such as vanishing or exploding gradients. In some embodiments, these issues may be mitigated by using variations of RNNs such as, for example, LSTMs or Gated Recurrent Units (GRUs), which are designed to handle long-term dependencies more effectively.

LSTM is a special type of RNN that is capable of learning long-term dependencies, making it effective in processing long sequences of data like lengthy text or audio. LSTMs may be utilized in the systems and methodologies disclosed herein in a manner similar to RNNs for sequential or time-series data. Because LSTMs are particularly good at capturing long-term dependencies in data, they may be useful for analyzing data over extended treatment periods.

As a specific, nonlimiting example of an embodiment of a system of the type described herein which utilizes an LSTM, the system collects sequential wellness data from the users over an extended period of time. This data may include, for example, physiological parameters, responses to previous PBM treatments, symptom ratings, and any other relevant metrics tracked over time. The time-series data may be preprocessed to handle missing values, normalize the data, and format it into sequences of a certain length, depending on the specific application. This may be an important step in embodiments utilizing LSTMs, since these networks are sensitive to the scale of input data. Additionally, the data may need to be segmented into windows (e.g., representing weeks or months) to facilitate the model training.

A LSTM model may be trained on these preprocessed sequences. Unlike traditional RNNs, LSTMs have a unique design that prevents the vanishing or exploding gradient problem during training, thus allowing them to learn from sequences that span long periods of time. The LSTM learns to recognize patterns over time that may signify improvement, stability, or deterioration in wellness outcomes.

Once the LSTM is trained, it may use new sequences of wellness data to predict future outcomes. For example, if a user's recent vital signs and symptom ratings follow a pattern that the LSTM has learned often leads to a worsening of symptoms, it might predict that the user's symptoms will worsen in the near future. These predictions may then be used to inform PBM treatment recommendations. For example, if the LSTM predicts a worsening of symptoms, the system may recommend adjusting the user's PBM treatment regimen to prevent this. Conversely, if the LSTM predicts stable or improving symptoms, it could recommend maintaining the current treatment plan. As with all machine learning models, the LSTM model may be periodically retrained or fine-tuned as new data is collected. This may help to ensure that the model's predictions remain accurate over time, even as users' wellness states and the treatments they receive evolve.

GRUs are a type of RNN that modifies the standard recurrent unit to make it easier to train and to help solve the vanishing gradient problem, a common issue in traditional RNNs. GRUs may be utilized in the systems and methodologies disclosed herein in a manner similar to LSTMs for processing sequential data. In some embodiments, their use may be preferable to LSTMs if computational efficiency is a concern, since GRUs are typically simpler and thus faster to train than LSTMs.

As a specific, nonlimiting example of an embodiment of a system of the type described herein which utilizes a GRU, the system collects sequential wellness data from the users over time. This data may include time-series data such as physiological parameters, symptom ratings, responses to PBM treatments, and any other metrics that change over time. The collected data is preferably preprocessed to handle missing values, normalize the data, and format it into sequences of a specific length depending on the specific use case. Preprocessing may be essential in some applications because the GRU, like all neural networks, is sensitive to the scale and quality of the input data.

Next, a GRU model would be trained on these preprocessed sequences. GRUs have a gating mechanism like LSTMs, but they use two gates (an update gate and a reset gate) instead of the three used in LSTMs (input, output, and forget gates). This makes them simpler and may expedite training. The GRU learns to recognize patterns in the sequential data that may indicate changes in wellness outcomes.

Once the GRU is trained, it may use new sequences of wellness data to predict future outcomes. For example, it may predict whether a user's symptoms are likely to worsen, improve, or remain stable in the future based on their recent data. These predictions may then be used to inform PBM treatment recommendations. If the GRU predicts a worsening of symptoms, for instance, the system may recommend a change in the PBM treatment plan. Conversely, if the GRU predicts stable or improving symptoms, the current treatment regimen may be maintained.

As more data is collected over time, the GRU model may be retrained or fine-tuned to ensure that its predictions remain accurate. This also allows the model to adapt to any changes in the users' wellness states or the treatments they receive.

RBFNNs use radial basis functions as activation functions. They have a first layer used for the data processing, and a second layer used for network output. RBFNNs may be utilized in the systems and methodologies disclosed herein, for example, for pattern recognition tasks, such as identifying clusters of users with similar wellness data or treatment responses. This information may be utilized to tailor treatment recommendations.

As a specific, nonlimiting example of an embodiment of a system of the type described herein which utilizes an RBFNN, the system collects wellness data from a plurality of users. This wellness data may include physiological parameters, symptom ratings, and responses to PBM treatments. The wellness data is preferably preprocessed to handle missing values, normalize the data, and potentially transform it into a suitable form for the RBFNN. For instance, the data might be converted into a suitable vector space representation.

An RBFNN is then trained on the wellness data. RBFNNs consist of an input layer, a hidden layer of radial basis functions, and an output layer. Each neuron in the hidden layer represents a “prototype” or “center”, and the neuron's output is a function of the distance between the input and this center. By learning appropriate centers and weights, the RBFNN may learn to recognize complex patterns in the data.

The trained RBFNN can be used to identify clusters of users with similar wellness data or treatment responses. This may be achieved by examining the hidden layer of the network. In particular, users whose data activate the same or similar hidden neurons could be considered part of the same cluster. Alternatively, the output of the RBFNN could be used as input to a clustering algorithm to group similar users.

The information about which cluster a user belongs to may then be used to tailor their PBM treatment recommendations. For instance, if a particular PBM treatment has been found effective for users in a certain cluster, it could be recommended for other users in that cluster. Similarly, treatments that have not been effective for a cluster could be avoided for users in that cluster.

The RBFNN model is preferably periodically retrained or fine-tuned as new data is collected. This allows the model to adapt to any changes in the user population, the wellness states, or the treatments being used.

DBNs are generative deep neural networks with many layers of hidden explanatory factors, often used for tasks such as dimensionality reduction, classification, regression, and collaborative filtering. DBNs may be utilized in the systems and methodologies disclosed herein, for example, for classification tasks (like determining the success of a treatment) or for reducing the dimensionality of the data, making it easier to work with.

As a specific, nonlimiting example of an embodiment of a system of the type described herein which utilizes a DBN in the context of a wellness system with PBM devices, the system collects wellness data from a plurality of users. This data may include physiological parameters, symptom ratings, responses to PBM treatments, and any other relevant metrics.

The collected data is preferably preprocessed to handle missing values, normalize the data, and format it appropriately for the DBN. A DBN may then be trained to perform dimensionality reduction by using it as an autoencoder. This may involve training the DBN to reproduce its input at its output (i.e., to approximate the identity function), but with a bottleneck layer that has fewer neurons than the input or output layers. The output of the bottleneck layer provides a lower-dimensional representation of the data.

Once trained, the DBN may be used to transform the high-dimensional wellness data into a lower-dimensional form. This simplified data may be easier to work with, while still being useful to inform PBM treatment recommendations.

The DBN may also be trained to perform classification or regression tasks. For example, the system may train a DBN to predict the success of a PBM treatment based on a user's wellness data and treatment objectives. The DBN may also be used to predict future wellness outcomes based on current data and treatment plans. The trained DBN may be utilized to make these predictions and evaluate their accuracy. The predictions could then be used to inform the PBM treatment recommendations.

A DBN can also be trained to perform collaborative filtering, which involves predicting a user's interests based on the interests of other similar users. For example, if a PBM treatment has been effective for users with similar wellness data and treatment objectives, it might be recommended for a given user. By using the DBN for collaborative filtering, the system may provide personalized PBM treatment recommendations that are tailored to each user's individual circumstances and needs. As new data is collected, the DBN is preferably periodically retrained or fine-tuned to ensure that its predictions remain accurate.

AEs are unsupervised neural networks used for learning efficient codings of input data. They are typically used for the purposes of anomaly detection or noise reduction. AEs may be utilized in the systems and methodologies disclosed herein, for example, for anomaly detection (like identifying unusual patterns in wellness data that might signal a problem) or for data denoising, which could be useful if the collected wellness data is noisy.

As a specific, nonlimiting example of an embodiment of a system of the type described herein which utilizes an AE in the context of a wellness system with PBM devices, the system collects wellness data from users, which might include physiological parameters, symptom ratings, and responses to PBM treatments. The data is preferably preprocessed to handle missing values, normalize the data, and transform it into a suitable format for the AE. The AE may then be trained on normal wellness data to learn a compressed representation of this data. It does this by trying to recreate the input data from this compressed representation, minimizing the difference (or “reconstruction error”) between the original data and the recreated data.

Once trained, the AE may be used for anomaly detection by comparing the reconstruction error for new data to a threshold value. Normal data should have a low reconstruction error (because the AE has learned to reproduce this type of data), while anomalous data should have a higher reconstruction error (because it differs from the normal data the AE was trained on). If a user's wellness data has a high reconstruction error, this may signal a problem such as an unusual response to treatment or a sudden change in wellness.

An AE may also be trained to perform data denoising by training it on noisy wellness data but using the corresponding clean data as the target output. This forces the AE to learn to remove the noise and recover the underlying clean data. The trained denoising AE may be used to clean up new wellness data that might be noisy. This may help improve the accuracy of the PBM treatment recommendations.

As more data is collected over time, the AE model may be retrained or fine-tuned to ensure that their anomaly detection and data denoising capabilities remain accurate. This also allows the models to adapt to any changes in the users' wellness states or the treatments they receive.

GANs are composed of two neural networks: the generator network, which creates new data instances, and the discriminator network, which tries to determine whether each instance of data it reviews belongs to the actual training dataset or was created by the generator network. GANs may be utilized in the systems and methodologies disclosed herein, for example, to generate synthetic data that's similar to the collected wellness data. This synthetic data could be used to augment the training data for the AI, potentially improving its performance.

As a specific, nonlimiting example of an embodiment of a system of the type described herein which utilizes a GAN in the context of a wellness system with PBM devices, the system collects wellness data from the users. This data may include physiological parameters, symptom ratings, responses to PBM treatments, and other relevant metrics. The collected data is preferably preprocessed to handle missing values, normalize the data, and format it appropriately for the GAN.

A GAN is trained on the collected wellness data. The generator network learns to produce synthetic data that resembles the real data, while the discriminator network learns to distinguish between the real data and the data generated by the generator network. Over time, the generator network gets better at producing realistic data, and the discriminator network gets better at spotting fake data.

Once trained, the generator network of the GAN can be used to create synthetic wellness data. This synthetic data should have similar statistical properties to the real data and can be used to augment the training data for other parts of the AI system. The synthetic data is combined with the real data to create an augmented training dataset. This augmented dataset is used to train the AI that's responsible for generating PBM treatment recommendations. By training on a larger and more diverse dataset, the AI may be able to learn more robust and generalizable patterns, potentially improving its performance. The AI, trained on the augmented dataset, may provide more robust PBM treatment recommendations based on the wider variety of data it has been trained on.

The GAN and the AI may be periodically retrained or fine-tuned as new data is collected. This ensures that the synthetic data remains representative of the real data, and that the AI's treatment recommendations remain accurate.

SOMs use unsupervised learning to produce a low-dimensional, discretized representation of the input space, typically used for dimensionality reduction or clustering tasks. SOMs may be utilized in the systems and methodologies disclosed herein, for example, for clustering users based on their wellness data or treatment objectives, which might be useful for personalizing treatment recommendations.

In such embodiments, the wellness data and treatment objectives of users may be collected. This may include a variety of data such as physiological measurements (like heart rate, skin temperature, etc.), genetic data, lifestyle factors (diet, exercise, stress levels), treatment objectives (such as reducing pain, improving mood, healing a skin condition), and responses to previous PBM treatments. Then, a SOM would be trained on this data. The goal of the SOM would be to identify clusters of users who have similar wellness data and treatment objectives.

To achieve this, the SOM would learn to map the high-dimensional data to a lower-dimensional grid (usually 2D) in a way that preserves the topological properties of the original data. Consequently, users who are similar to each other in terms of their wellness data and treatment objectives would be mapped to nearby locations on the grid. Once the SOM has been trained, it may be used to cluster the users. Each node on the grid would represent a cluster, and all the users mapped to that node may be considered part of the same cluster. These clusters may then be used to inform the PBM treatment recommendations provided by the system. For example, if it's found that users in a certain cluster respond particularly well to a specific type of PBM treatment, then that treatment may be recommended to other users in the same cluster. Or if the treatment objectives of the users in a cluster are found to be particularly challenging to achieve, additional resources or alternative therapies could be suggested. Moreover, as new wellness data is collected, the SOM can be updated, ensuring that the user clusters and the corresponding treatment recommendations remain accurate and relevant.

It will be appreciated that the foregoing is a high-level overview of how SOMs could be used in this system. Particular embodiments may require careful consideration of factors such as how to preprocess the data, the size and layout of the SOM, and the specific algorithm used for training the SOM (e.g., the Kohonen algorithm).

Various types of wellness data may be utilized in the systems and methodologies disclosed herein. These may include, without limitation, biometric data (such as, for example, heart rate, blood pressure, body mass index (BMI), cholesterol levels, blood glucose levels, and other vital signs); physical activity data (such as, for example, the number of steps taken per day, amount of vigorous or moderate exercise, types of physical activity, and sedentary time); sleep data (such as, for example, total sleep time, sleep efficiency, number and duration of awakenings, and stages of sleep); nutrition data (such as, for example, information about diet such as caloric intake, macronutrient distribution, hydration, and consumption of fruits, vegetables, or other specific foods); stress levels (such as, for example, self-reported stress levels or physiological indicators of stress such as cortisol levels); mental health data (such as, for example, self-reported mood, anxiety levels, depressive symptoms, and cognitive function); social wellness data (such as, for example, self-reported data about social activity, relationship satisfaction, feelings of loneliness or connection, and involvement in the community); substance use data (such as, for example, consumption of alcohol, tobacco, caffeine, or other substances); medical history (such as, for example, past diagnoses, surgical history, medication use, and family medical history); genomic data (such as, for example, data derived from genetic testing, such as genetic predispositions to certain diseases); environmental data (such as, for example, data about the individual's living and working conditions, exposure to pollutants or allergens, and access to green spaces); occupational data (such as, for example, job satisfaction, work-related stress, ergonomic factors, and occupational hazards); self-reported symptoms (such as, for example, data about pain, fatigue, digestive issues, and other symptoms); immunization record (such as, for example, information about vaccinations received); health screening results (such as, for example, the results from health screenings such as mammograms, colonoscopies, skin checks, and the like); sexual health data (such as, for example, information about sexual activity, practices, and sexual health conditions or concerns); allergies (such as, for example, information about food, drug, environmental, or other allergies); menstrual cycle data (such as, for example, information about the menstrual cycle, symptoms, and related conditions (like PCOS or endometriosis)); bone density (such as, for example, information related to bone health, especially relevant for older users); and mindfulness and meditation practices (such as, for example, information about frequency, duration, and types of mindfulness or meditation practices).

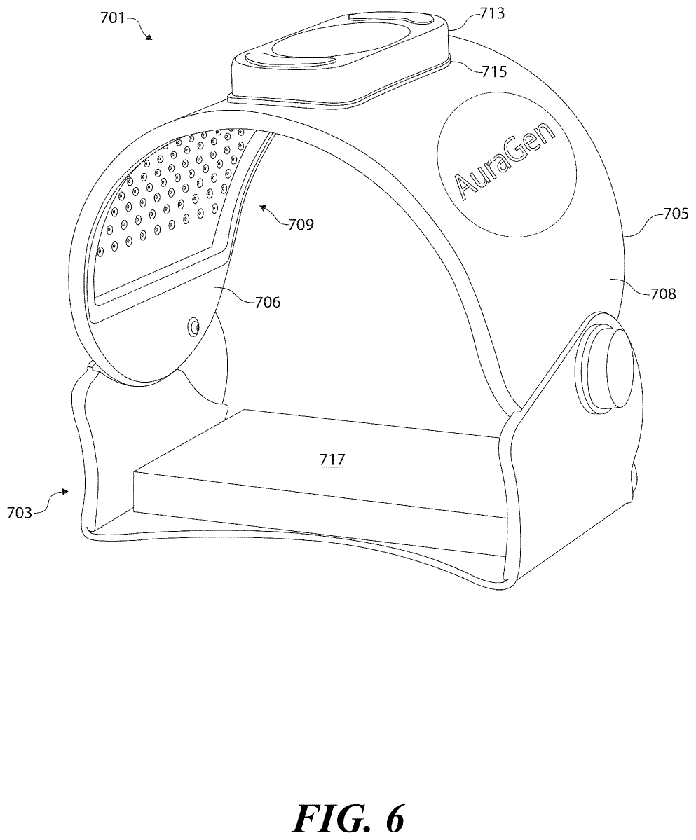

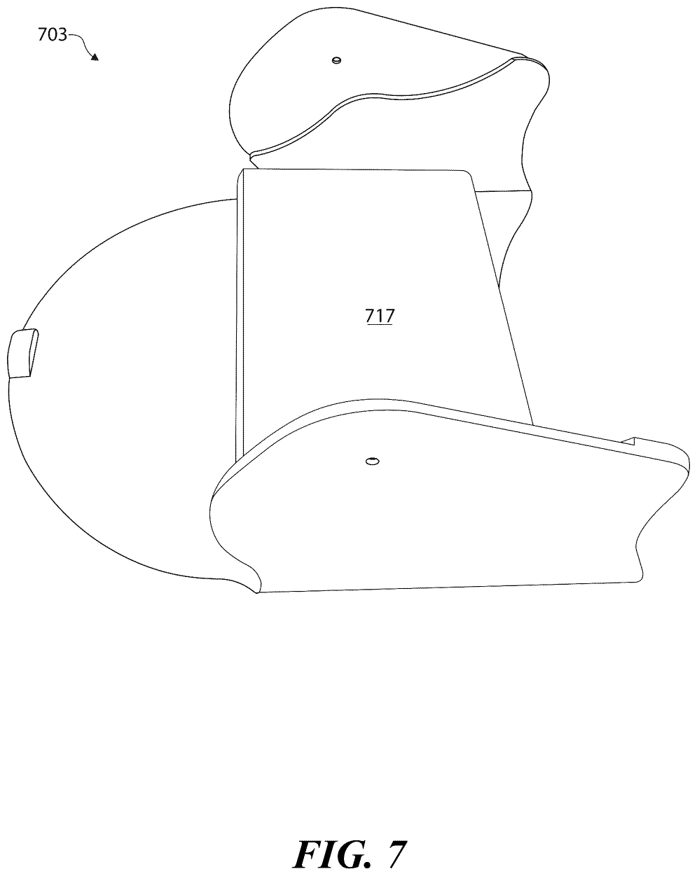

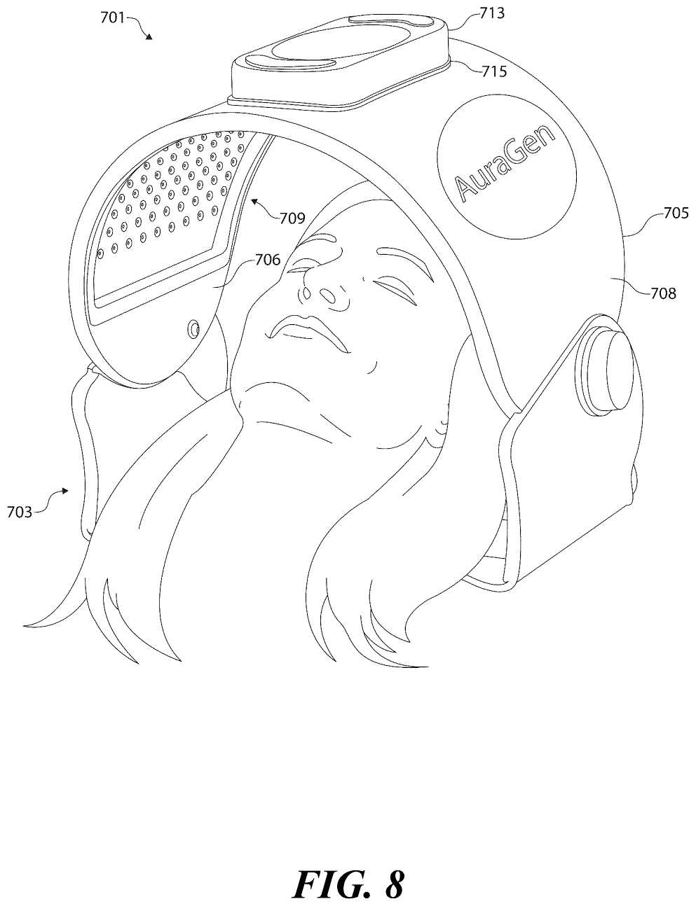

Various PBM devices may be utilized to implement the wellness optimization systems and methodologies disclosed herein. FIGS. 6-9 illustrate a particular, nonlimiting embodiment of such a device. The PBM device 701 depicted therein comprises a base 703 (shown in isolation in FIG. 7) having a peripheral element 705 attached thereto and, optionally, an audio headset (not shown; the need for a headset may be determined, for example, by whether the light therapy device implements one or more entrainment methodologies that utilize traveling waves originating from the same source, or standing waves generated by two distinct sources). The base 703 and peripheral element 705 define an opening 707 in which a user's head is placed (see FIG. 8). The base 703 and/or peripheral element 705 may be equipped with an audio jack, a Bluetooth transmitter, or other suitable provisions as necessary or desirable to support the use of an audio headset by the user.

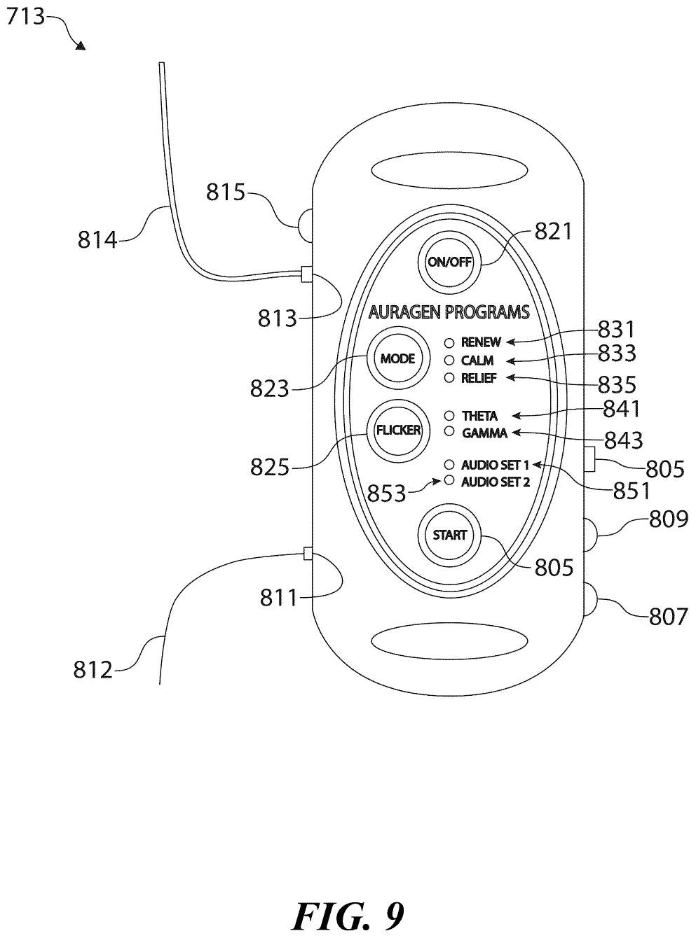

The base 703 in this particular embodiment is equipped with a pillow 711 for user comfort, and to provide the user with the ability to lie down or sleep during a light therapy session. The peripheral element 705 has a first major inward-facing surface 706 and a second major outward-facing surface 708. The first major surface 706 is equipped with an LED array 709 which can be activated with a remote control 713 to illuminate the user's head at one or more wavelengths. The second major surface 708 is equipped with a holder 715 for the remote control 713. The remote control 713, which is shown in greater detail in FIG. 9, may also be utilized to activate or modulate a light therapy program implemented by the device, or to activate or modulate light emitted by the LED array 709, to select one or more wavelengths of light emitted by the LED array 709, and to control the playback of one or more audio files or tracks.

In use, a user's head is placed in the opening 707 such that the back of the user's head is on the pillow 711 and such that the user is facing the first major surface 706 of the peripheral portion 705 as shown in FIG. 8. The user (or possibly a clinician or other assistant) then uses the remote control 713 to activate the therapy device 701 and to cause it to function in one or more selected modes. Regarding the latter, it is to be noted that the therapy device 701 may be programmed with various algorithms which cause it to function in particular ways, some of which are described in greater detail below. The therapy device 701 may also be programmed to play music or soundtracks, which may be advantageously matched to the particular algorithm being implemented by the therapy device 701.

In some embodiments, the therapy device may include a port to allow plugin of additional LED portable devices that may be place in the mouth of the user (via, for example, a mouth guard). In other embodiments, the therapy device may include a small pad that may be wrapped or directly applied to a specific body part of the user. In other embodiments, the therapy device may include pads that induce vibration into the body of the user which may be laid under the body or directly applied to specific body part of the user. In still other embodiments, the therapy device may include a set of googles or glasses that are placed over the eyes of the user to provide focused treatment to those areas, or to prevent treatment of those areas. Of course, it will be appreciated that any of the foregoing accessories may be utilized in combination in various embodiments of the systems and methodologies disclosed herein.

Various LEDs 709 or other light sources which emit at various wavelengths may be utilized in the devices and methodologies disclosed herein. However, the use of light sources which emit at wavelengths in the red, infra-red and blue-turquoise regions of the spectrum are preferred, and the use of light sources which emit at about 470 nm, 670 nm and 870 nm are especially preferred. In a preferred mode of operation, these light sources are made to oscillate or flicker in the theta or gamma band.

It will be appreciated that light may be emitted at the foregoing wavelengths in various manners, including sequentially or simultaneously. For example, the LED array 709 may be operated to emit electromagnetic radiation at a single wavelength (i.e., monochromatically) or at multiple wavelengths. In some cases, the LED array 709 may include a first set of LEDs that are operated to emit light at a first wavelength, a second set of LEDs that are operated to emit light at a second wavelength, and (optionally) a third set of LEDs that are operated to emit light at a third wavelength. In other cases, the LED array 709 may be operated such that all of the LEDs in the array emit light at a first wavelength for a first period of time, all of the LEDs in the array emit light at a second wavelength for a second period of time, and (optionally) all of the LEDs in the array emit light at a third wavelength for a third period of time.

The particular wavelength(s) of emission of the LED array 709, the duration of those emissions, the frequency of oscillation (if any), the intensity of the emitted light, the selection of accompanying audio tracks or files (if any), and/or the oscillation of any accompanying audio tracks, files or component(s) thereof, may be selected to achieve a desired physiological or psychological effect. It will be appreciated that, in some embodiments, the duration of emission for any particular wavelength of light may remain constant or may vary during the course of a therapy session. It will further be appreciated that, in some embodiments, any of the LEDs in the LED array 709 may be operated to emit two or mor wavelengths of light, including broadband radiation or white light.

The therapy devices and methodologies disclosed herein may be utilized as an effective tool in treating a subject for certain psychological or physiological conditions, or for prevention of these conditions. These conditions include, but are not limited to, traumatic brain injury, addiction or dependence (including, for example, addiction to, or dependence on, opioids, amphetamines, stimulants, alcohol or cannabis), depression (and more specifically, clinical depression or major depression), PTSD, developmental trauma disorder, traumatic brain injury and its sequelae, and Alzheimer's disease. In a preferred embodiment of the methodology disclosed herein, a subject is first diagnosed as suffering from one of the foregoing conditions, and then brainwave entrainment is utilized to treat the subject.

Various aspects of the systems and methodologies described herein have been described above with respect to the particular, non-limiting embodiments disclosed herein. It will be appreciated that these various aspects may be employed in various combinations (including various sub-combinations) or permutations in accordance with the teachings herein.

For example, while the use of light sources which emit at wavelengths in the red, infra-red and blue-turquoise regions of the spectrum are preferred, and the use of light sources which emit at about 470 nm, 670 nm and 870 nm are especially preferred, it will be appreciated that the devices and methodologies disclosed herein may utilize various other frequencies or wavelengths of electromagnetic radiation to achieve desired physiological or psychological effects. These wavelengths or frequencies may be selected, for example, from the visible, infrared or ultraviolet regions of the electromagnetic spectrum.

Similarly, in a preferred mode of operation, the intensities of one or more of these light sources are made to oscillate or flicker in the theta or gamma frequency band during at least a portion of a therapy session. However, embodiments are possible in which the light sources are made to oscillate or flicker at other frequencies, or in which the light sources (or elements thereof) operate in a manner which is not time varying. Embodiments are also possible in which the light sources are made to oscillate or flicker at harmonics of the foregoing frequencies.

While the embodiment of FIGS. 6-9 is a preferred embodiment of the therapy device described herein, it will be appreciated that therapy devices of various shapes, configurations, layouts and functionalities may be utilized in the practice of the methodologies disclosed herein, and these therapy units may be provided with various accessories.

For example, in some embodiments, therapy devices may be utilized that are adapted to illuminate one or more inner surfaces of a subject's oral cavity. In such embodiments, a light therapy unit utilized for this purpose may be fashioned as a standalone device, while in other embodiments, such a light therapy unit may be fashioned as an accessory to a main light therapy unit which is utilized to illuminate the outer surfaces of a subject's head. In embodiments of the latter type, the accessory may be adapted to communicate with the main light therapy device such that the accessory is controlled by, or acts in concert with, the main light therapy device.

In some instances of embodiments of a brainwave entrainment device adapted to illuminate one or more inner surfaces of a subject's oral cavity, the light therapy unit may be equipped with a mouth guard which is in optical communication with a light source by way of a suitable light guide, and which distributes light received from the light source in a suitable manner. In some cases, the mouthguard may be customized to the user. By way of example but not limitation, such a mouth guard may be adapted to direct suitable wavelengths of light to various surfaces of the oral cavity of a subject, including the teeth, gums, upper or lower mouth, and throat. The mouth guard, light guide or portions thereof may be equipped with suitable materials that specularly or diffusely transmit or reflect incident radiation in one or more directions. In addition to their possible use in treating physiological or psychological conditions, these embodiments may offer additional benefits such as, for example, the treatment or prevention of gingivitis and other bacterial infections.

In some embodiments of the devices disclosed herein, measures may be taken to ensure that the light therapy device is applied to only specific parts of the user's body. For example, in some embodiments, the aforementioned light therapy unit which is adapted to illuminate one or more inner surfaces of a subject's oral cavity may be used by itself such that only these surfaces are exposed to the brainwave entrainment therapy. Similarly, in some embodiments, the user may be equipped with glasses or goggles such that the user's eyes or optical nerves are not exposed to the brainwave entrainment light, or such that this light is concentrated on the user's eyes or optical nerves. In still other embodiments, an optical pad or other suitable means may be utilized to apply light therapy only to the back of a user's neck, or to a user's chest (alone or in combination with the application of light therapy frequencies to the user's head).

Preferred embodiments of the devices disclosed herein are adapted to allow the user to lie down or otherwise assume a state of repose during a light therapy session. Such embodiments may include, for example, a pillow or one or more deformable pads which support the user's head during light therapy. Here, it is notable that many other devices in the art which are designed for light therapy require the user to remain in a sitting or standing position for the duration of the therapy.

In some embodiments of the devices disclosed herein, the device may be equipped with a suitable controller, which may be wireless or wired. The controller may be programmable or pre-programmed, and may be equipped with suitable programming instructions (which may include an operating system) recorded in a tangible, non-transient medium that cause the brainwave entrainment or stimulation device to operate in various modes or to perform various functions. These modes or functions may be selected or optimized for the treatment of various portions of a subject's body, or for the treatment of particular physiological or psychological conditions.

Various parameters (and ranges of these parameters) may be utilized in the therapy devices and methodologies disclosed herein. These include, without limitation, wavelength, frequency, entrainment waveform, energy, fluence, power, irradiance, intensity, pulse mode, treatment duration, and repetition. These parameters and their values may be selected to treat a subject for certain psychological or physiological conditions, to lessening the severity or effects of these conditions, and/or to preventing the occurrence of these conditions. These conditions include, but are not limited to, traumatic brain injury, opioid addiction (including, for example, heroin addiction or addiction to prescription opioids), alcohol misuse disorder or alcohol dependence, nicotine dependence or addiction, depression (and more specifically, clinical depression or major depression), mild cognitive impairment, dementia, Alzheimer's disease, attention deficit disorder, developmental trauma disorder, and autism.

It will be appreciated that the light therapy devices disclosed herein, and the components thereof, may be equipped with suitable optical elements to achieve various purposes. Such optical elements (or portions thereof) may be diffusely or specularly reflective or transmissive. Suitable optical elements may include, but are not limited to, reflective elements, polarizers, color shifting elements, filters, light guides (including, without limitation, optical fibers, light pipes and waveguides), prismatic elements, lenses (including Fresnel lenses), and lens arrays.

In preferred embodiments of the systems and methodologies disclosed herein, one or more audio tracks or audio files may be provided that may be modulated, coordinated and/or synchronized with the plurality of LEDs or the light emitted therefrom. Preferably, the audio tracks or audio files include sound that is modulated, coordinated and/or synchronized with the LEDs or the light emitted therefrom at one or more frequencies selected from the group consisting of gamma, beta, alpha, theta and delta frequencies. The audio tracks or files (alone, or in combination with any light wavelengths utilized) may be selected to achieve a desired physiological or psychological effect in the user, either alone or in combination with the light therapy.

In preferred embodiments of the systems and methodologies disclosed herein, one or more audio tracks or audio files may be provided that may be modulated, coordinated and/or synchronized with the plurality of LEDs or the light emitted therefrom. Preferably, the audio tracks or audio files include guided meditation and mindfulness training, coordinated and/or synchronized with the LEDs or the light emitted therefrom at one or more frequencies selected from the group consisting of gamma, beta, alpha, theta and delta frequencies. The audio tracks or files (alone, or in combination with any light wavelengths utilized) may be selected to achieve a desired physiological or psychological effect in the user, either alone or in combination with the light therapy.

In preferred embodiments of the systems and methodologies disclosed herein, one or tactile stimulation to include vibration pads that may be modulated, coordinated and/or synchronized with the plurality of LEDs or the light emitted therefrom. Preferably, the vibrations or tactile stimulation is coordinated and/or synchronized with the LEDs or the light emitted therefrom at one or more frequencies selected from the ranges depicted in FIG. 6. The vibration frequencies (alone, or in combination with any light wavelengths utilized) may be selected to achieve a desired physiological or psychological effect in the user, either alone or in combination with the light therapy. The tactile stimulation variation can include frequency, intensity, pulse mode, treatment duration, and repetition. The tactile stimulation can be made to parts of the body to include fingers, hands, feet, or back.

One skilled in the art will further appreciate that the systems and methodologies disclosed herein may be used not only to treat various physiological or psychological conditions, but to prevent them from occurring in the first place. For example, these systems and methodologies may be adapted to prophylactically prevent the onset of depression, PTSD, ADHD, opioid addiction (for example, heroine or oxycodone), or conditions resulting from traumatic brain injury, or of conditions which might otherwise result from the foregoing.

The systems and methodologies disclosed herein may be utilized in conjunction with other methodologies or techniques. For example, these systems and methodologies may be used in combination with emotional freedom technique (EFT) tapping. EFT tapping is a holistic healing technique that may be utilized to treat various issues including, without limitation, stress, anxiety, phobias, emotional disorders, chronic pain, addiction, weight control, and limiting beliefs. EFT tapping involves tapping with the fingertips on specific meridian endpoints of the body, while focusing on negative emotions or physical sensations. Proponents of the method claim that it calms the nervous system, rewires the brain to respond in healthier ways, and restores the body's balance of energy.

One skilled in the art will further appreciate that the optimal parameters for a therapy session may depend on a variety of factors including, but not limited to, the condition being treated (or prevented), the physiological or psychological state of the user, the user's biometrics, and other such factors. In some use cases, selection of these parameters may be made by, or in coordination with, a physician, a psychiatrist, or other healthcare provider. These parameters may include, but are not limited to, the wavelengths of light to be utilized, the audio tracks or files to accompany the light therapy, the frequencies of oscillation utilized for the intensity in any of the wavelengths or light or sound, the portions of the user's head or body to be exposed to the light therapy, and the duration of the treatment.

While the devices and methodologies disclosed herein have frequently been described with reference to the use of traveling waves originating from a common source, one skilled in the art will appreciate that various embodiments of these methodologies and devices may also be produced which utilize waves originating from distinct sources (e.g., standing waves). In some embodiments, various devices, materials or other such measures may be taken to cause or prevent reflection of the therapeutic waves.

The devices and methodologies disclosed herein have frequently been described or illustrated with respect to PBM devices. However, it is to be understood that these devices and methodologies may have many uses in other fields and applications. These include, without limitation, their use in photic stimulation, including intermittent photic stimulation.

Since some embodiments of the systems and methodologies disclosed herein may collect, process, or store health-related information, various measures may be taken to ensure that these systems and methodologies are HIPPA compliant. HIPAA (the Health Insurance Portability and Accountability Act of 1996) is a U.S. federal law that was created to improve the efficiency and effectiveness of the healthcare system. One of the key aspects of HIPAA is that it established standards for the electronic exchange, privacy, and security of health information.

The privacy component of HIPAA, also known as the Privacy Rule, protects the medical records and other personal health information of individuals. It requires that healthcare providers and other covered entities use and disclose protected health information only for certain permitted purposes and requires these entities to give patients certain rights with respect to their health information. The security component of HIPAA, or the Security Rule, establishes a national set of security standards for protecting health information that is held or transferred in electronic form. It requires entities to implement administrative, physical, and technical safeguards to ensure the confidentiality, integrity, and availability of electronic protected health information. Of course, maintaining compliance is an ongoing process that may require regular reviews and updates as technology and regulations change.

One feature which may be utilized in the systems and methodologies described herein to ensure HIPPA compliance is data encryption. In particular, in the systems and methodologies described herein, all wellness data is preferably encrypted in transit and at rest to protect sensitive information. This includes data sent from client devices to the server, and data stored in the wellness database.

Access controls are another feature which may be utilized in the systems and methodologies described herein to ensure HIPPA compliance. In particular, measures may be taken in these systems and methodologies to ensure that only authorized individuals have access to the wellness database. This includes implementing strong user authentication methods and keeping detailed access logs.

Another feature which may be utilized in the systems and methodologies described herein to ensure HIPPA compliance is data integrity. In particular, the systems disclosed herein will preferably be adapted to ensure the integrity of the data it holds. This may involve maintaining backups and implementing procedures to prevent and detect data tampering.

Audit trails are another feature which may be utilized in the systems and methodologies described herein to ensure HIPPA compliance. Such audit trails will typically comprise the maintenance of detailed logs of all data access and changes to allow tracking of who accessed what data and when. It will be appreciated that, in some applications, such audit trails may be crucial for accountability and for conducting audits.

Secure interfaces are also preferably utilized in the systems and methodologies described herein to ensure HIPPA compliance. In particular, it is preferred that both the user and administrative interfaces are secure and implement best practices (such as, for example, SSL) for data transmission and strong authentication methods.

Business associate agreements (BAAs) may also be utilized in the systems and methodologies described herein to ensure HIPPA compliance. In particular, the use of BAAs is preferred any time third-party services are used for processing or storing wellness data. Such a contract ensures that the third party will also comply with HIPAA regulations.

Another feature which may be utilized in the systems and methodologies described herein to ensure HIPPA compliance is risk analysis and management. In particular, it is preferred that regular security risk assessments are performed to identify potential risks and vulnerabilities to the confidentiality, integrity, and availability of the wellness data. Once these risks are identified, proper security measures may be implemented to manage them.

Where applicable, employee training is also preferably utilized in the systems and methodologies described herein to ensure HIPPA compliance. In particular, all employees who have access to the wellness data should be properly trained on HIPAA regulations and company policies regarding data privacy and security.

Where applicable, the systems and methodologies described herein may also have an incident response plan associated with them to ensure HIPPA compliance. This requires that any organization associated with these systems and methodologies has a plan in place to respond to security incidents, including data breaches. This plan includes steps to be taken to contain the incident, mitigate damage, notify affected individuals, and report the breach to the Office for Civil Rights (OCR).

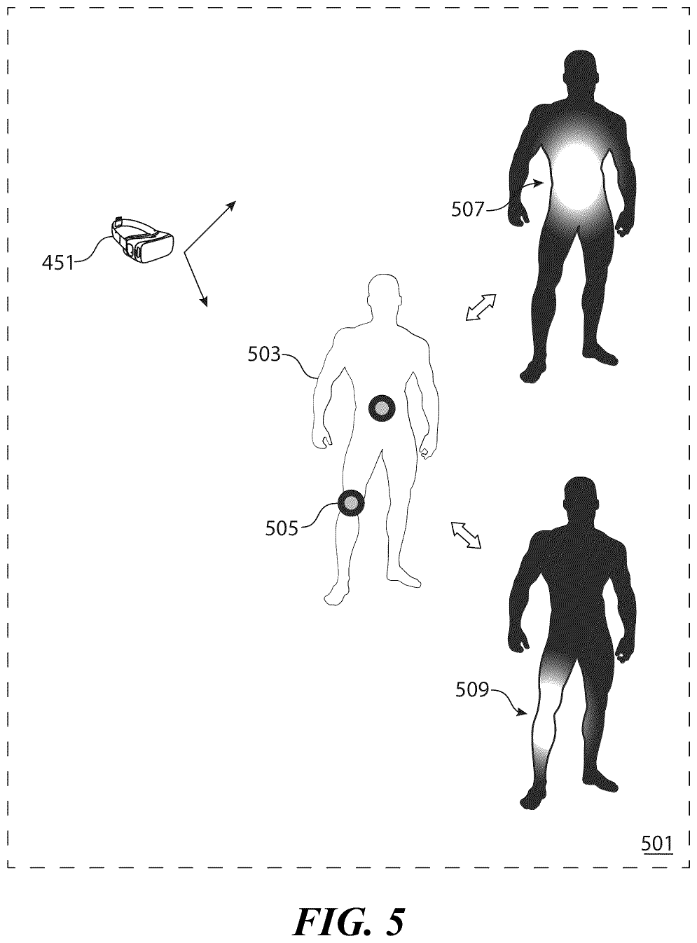

While FIGS. 5-8 illustrate a particular embodiment of a PBM device which may be utilized in the systems and methodologies described herein, it will be appreciated that a variety of PBM devices may be utilized in these systems and methodologies. A typical embodiment of such a device will include a light source, a power supply, control circuitry, sensors, a microprocessor or microcontroller, memory, a communication module, a user interface, safety features, and a suitable casing or chassis.

Since PBM relies on the use of light to promote healing and wellness, the PBM device will include a light source may comprise LEDs (Light Emitting Diodes) or lasers, depending on the specific type of therapy being administered. The light source may be tunable to emit specific wavelengths of light that are used in PBM therapy (for example, in the red and near-infrared spectrum), or may include a plurality of light sources, each of which emits light at a particular wavelength or emission band.

To operate the light source, the PBM device requires a power supply. This may be, for example, a direct connection to an electrical outlet or a rechargeable battery for portable devices.

The PBM device will also typically be equipped with control circuitry to regulate the operation of the light source. The control circuitry may control such parameters as, for example, the intensity, pulse, duration, and wavelength of light utilized. The control circuitry may also handle safety features like timers or automatic shutoff mechanisms.

The PBM device may also be equipped with one or more sensors to collect wellness data. Such sensors may include, for example, photodetectors, temperature sensors, heart rate monitors, blood oxygen saturation sensors (SpO2 sensors), skin conductance sensors, accelerometers, gyroscopes, cameras, spectrometers, skin moisture sensors, bio-impedance sensors, pressure sensors, blood pressure sensors, glucose monitors/sensors, UV sensors, body temperature sensors, respiration sensors, galvanic skin response (GSR) sensors, optical heart rate sensors, spectroscopy sensors, sweat sensors, sleep trackers, motion/activity sensors, ambient light sensors, environmental sensors, ECG sensors, and cameras (e.g., to monitor skin response).

Thus, for example, photodetectors may be utilized to measure the intensity of the light emitted during PBM treatment sessions. This information could be used to adjust the treatment protocol if necessary and ensure the consistency of the light therapy treatment. Skin moisture sensors may be used to measure the hydration level of the user's skin. This may be relevant in determining the effectiveness of the PBM treatment, especially if one of the treatment objectives is to improve skin health. A heart rate monitor may be utilized to measure the user's heart rate before, during, and after the PBM treatment. Changes in heart rate may indicate how the body is responding to the treatment. An SpO2 sensor may be utilized to measure the level of oxygen saturation in the blood. This may provide valuable information about the user's overall health and how they are responding to PBM treatment. Blood pressure sensors may be included to monitor the user's blood pressure levels, providing valuable data for cardiovascular health and stress management.

Body temperature sensors may also be utilized in the PBM devices described herein. By tracking the body's surface temperature, such sensors may provide insights into metabolic changes, illness, or inflammation, which might influence the treatment objectives or effectiveness of the PBM therapy. Similarly, respiration sensors may be utilized to monitor the user's breathing rate, thus possibly providing insights related to stress, sleep quality, or cardiovascular health. Optical heart rate sensors may be utilized to monitor the user's heart rate non-invasively, providing important data for cardiovascular health, stress management, or monitoring the body's response to therapy. Pulse oximetry sensors may be utilized to measure the oxygen saturation level in the user's blood, which could be valuable for users with respiratory conditions or for monitoring overall respiratory health. Bioimpedance sensors may be utilized to measure the electrical impedance of body tissues, which may provide valuable information about body composition, including levels of body fat and muscle mass.

A skin conductance sensor may be included to measure changes in the skin's ability to conduct electricity, which can be influenced by sweat secretion due to stress or other physiological changes. These may include galvanic skin sensors (GSRs) to measure the electrical conductance of the skin, which typically varies with its moisture level. This may be utilized as an indicator of psychological or physiological arousal, including stress or anxiety levels. Similarly, spectroscopy sensors may be utilized to analyze the interaction of light with the user's skin and underlying tissues, potentially providing valuable information about skin health, hydration, or the effectiveness of the PBM therapy. Sweat sensors may be utilized to analyzes sweat, which may provide useful data about hydration levels, electrolyte balance, and potentially certain metabolic functions. Sleep trackers, which may include a sensor or a suite of sensors, may be utilized to track sleep patterns (like REM cycles) that may provide important information about a user's overall wellness and response to PBM therapy. ECG sensors may be utilized to monitor the electrical activity of the heart, providing detailed data about heart health and potentially alerting users and healthcare providers to heart-related issues.

An accelerometer or gyroscope may be included in the PBM device. This may provide data about the user's movement and activity levels, which could be relevant for certain wellness goals. Bio-impedance sensors may be utilized to measure the resistance of body tissue to the flow of an electric current. This may provide information about the user's body composition, such as the proportion of fat to muscle, which may be useful for certain wellness objectives. Pressure sensors may be used to measure the force exerted by the user on the PBM device. This might be relevant in applications where the device needs to be held against the skin with a certain amount of pressure to ensure effective treatment. A UV sensor may be included to measure the levels of ultraviolet light exposure the user is receiving. This may help the user avoid excessive UV exposure, which can be harmful to the skin and overall health.

Environmental sensors such as humidity, temperature, or air quality sensors may be included to measure the conditions in the user's environment. These factors may potentially impact the effectiveness of PBM treatment, thus making it desirable to account for these factors when generating PBM recommendations. Similarly, ambient light sensors may be utilized to measure the level of light in the user's environment, which may impact the effectiveness of PBM therapy.

In some embodiments of the PBM devices disclosed herein, a (preferably non-invasive) glucose sensor may be utilized to monitor the user's blood sugar levels. This may be particularly useful for users with diabetes, enabling them to monitor their glucose levels and adjust their PBM treatment accordingly. In some embodiments of the PBM devices disclosed herein, a camera or spectrometer might be used to assess skin conditions or measure changes in skin coloration due to the PBM treatment.

Each of the foregoing sensors may be carefully integrated into the PBM device (or a device associated therewith) and may be connected to the microcontroller. The microcontroller will typically be responsible for collecting the sensor data, possibly processing it, and then transmitting it via the communication module to the server for further processing and analysis by the artificial intelligence engine. All the collected sensor data may contribute to the wellness data of the user, allowing for personalized PBM recommendations based on the individual's specific needs and responses to the treatment.

The PBM device may also be equipped with a microprocessor or microcontroller to process signals from the sensors, control the light source, and handle communication with the software client. The microprocessor or microcontroller forms the heart of the PBM device, managing the execution of software code and interacting with other components of the system.

A microprocessor is essentially a central processing unit (CPU) on a single integrated circuit (IC). It is responsible for performing all the primary arithmetic and logical operations in a device. Key components within a microprocessor include the Arithmetic Logic Unit (ALU) for calculations, control unit for directing data flow, and registers for temporary data storage. It will be appreciated that a microprocessor typically requires external components such as memory and input/output peripherals to implement its functionality.

In the PBM devices disclosed herein, a microprocessor may be especially desirable if the device needs to handle complex tasks, run a full-featured operating system, or manage multiple peripherals, due to their capability of executing intricate software applications with great efficiency.

Microcontrollers may also be utilized in the PBM devices disclosed herein. Unlike microprocessors, microcontrollers are compact integrated circuits designed for specific operations in an embedded system. They typically include a CPU, memory (RAM, ROM), and I/O peripherals (timers, communication ports), all on the same chip. This configuration makes microcontrollers more self-contained than microprocessors, but also more specialized. A microcontroller may be a more suitable choice for the PBM device if the device's operations are relatively simple, deterministic, and real-time, with low power consumption requirements. Tasks might include managing sensor data, controlling the light source, or communicating with the server via the communication module.

The microprocessor or microcontroller in a PBM device may have a wide range of responsibilities. These may include controlling the intensity, frequency, and timing of the light source based on the received treatment recommendations; reading data from any sensors on the device, such as skin temperature or light intensity sensors; communicating with the server, including sending wellness data and receiving treatment recommendations; managing the user interface, such as updating displays or responding to button presses; implementing safety features, such as shutting down the light source if a sensor detects an unsafe condition; and processing user inputs, like their treatment objectives and feedback. It will be appreciated that whether a microprocessor or microcontroller is the best choice for the PBM device will depend on the specific requirements of the device's design and intended functionality.

The PBM device will typically require memory to store firmware, temporarily store data before transmission, or even to record treatment history. The memory is essentially an electronic component where data is stored. Memory is one of the primary elements of any computing device, and it forms a fundamental part of the device's operation. In the context of a PBM device, memory can be categorized into two main types: volatile memory (RAM) and non-volatile memory (storage memory).

Random Access Memory (RAM) is a form of volatile memory that provides temporary storage and working space for the device's operating system and applications. When the PBM device is switched off, all data stored in the RAM is lost. In the context of the PBM device, the RAM may be used to store temporary data such as, for example, current session information, user input, intermediate calculations, or the like, which are needed for immediate operations and computations. By contrast, non-volatile memory (storage memory) may be utilized as the permanent storage area in the PBM device where user data, application software, firmware, treatment protocols, wellness database, and other data are stored. This type of memory retains the information even when the device is switched off. Examples of non-volatile memory which may be utilized in the PBM devices described herein include Flash memory and EEPROM (Electrically Erasable Programmable Read-Only Memory).

In some embodiments, the memory component may also include a cache memory that serves as an intermediate stage between ultra-fast CPU registers and much slower main memory. In such embodiments, the provision of a cache memory may speed up data access times and improve overall system performance.

The memory may also be equipped with adequate security measures to protect sensitive user data, in line with regulations such as HIPAA. This may include encryption of data at rest, secure access controls, and mechanisms to prevent data tampering. The capacity and speed of the memory component may need to be chosen based on the requirements of the specific PBM device, the complexity of the software and algorithms it runs, and the amount of data it needs to store and process.

In order to communicate with the software client installed on a user device, the PBM device is preferably equipped with a communication module. The communication module may be, for example, a Wi-Fi or Bluetooth module for wireless communication, or a USB or Ethernet port for wired communication. The communication module allows the PBM device to transmit and receive data to and from the server or other devices, such as a user's smartphone or computer. The specific composition of the communication module may vary depending on the desired functionality and specifications of the PBM device. However, in a preferred embodiment, it will contain a transceiver, an antenna, a modulator/demodulator, a microcontroller, memory, a power management unit, and interface hardware. Various pre-made communication modules (such as, for example, Wi-Fi or Bluetooth modules) are commercially available that can be integrated into the design of the PBM device. Such commercially available modules are typically equipped with all the necessary components and are usually easy to interface with, since they tend to have well-documented application programming interfaces (APIs). The communication module will preferably support encryption and security protocols to protect the data being transmitted, especially considering the sensitive nature of wellness data and the potential need for HIPPA compliance. The communication module will also preferably support various networking protocols (such as, for example, TCP/IP) to allow communication over the Internet and other networks.

The communication module is preferably equipped with a transceiver. The transceiver enables the PBM device to participate in the wellness system by facilitating robust, reliable, and secure communication with the server. It is equipped with both a transmitter and a receiver which are combined and share common circuitry or a single housing. The type of transceiver utilized may depend on the specific wireless technology being employed such as, for example, Wi-Fi, Bluetooth, or potentially even cellular networks. In the PBM devices described herein, the transceiver typically functions as the core component of the communication module. It enables the bidirectional transfer of data between the PBM device and the server. The transceiver sends wellness data and treatment objectives from the user to the server and receives light therapy treatment recommendations from the server. The transceiver will typically be in constant communication with the microprocessor/microcontroller which controls these data operations.

In transmission mode, the transceiver sends data to the server. The microprocessor or microcontroller passes the wellness data and treatment objectives to the transceiver, which converts this data into a suitable format for wireless transmission. This may involve, for example, modulating the data onto a carrier signal. In reception mode, the transceiver receives data from the server. This typically involves demodulating the incoming signal to extract the transmitted data, and passing this data to the microprocessor or microcontroller. The data will typically include treatment recommendations generated by the server's AI engine.

The transceiver will typically operate on specific frequency bands. In the context of the PBM device, the transceiver will typically operate in a band suitable for short-range, low-power communication, such as the 2.4 GHz band used by Bluetooth and Wi-Fi devices. The transceiver will typically need to support the communication protocols used by the server. This may include standard protocols such as Wi-Fi (for a local network) or LTE/5G (for a wide-area network), or proprietary protocols designed specifically for the wellness system.

In some embodiments, the PBM device may be a portable device running on battery power. In such embodiments, the transceiver may be designed to conserve power as much as possible. This may involve the implementation of techniques such as putting the transceiver into a low-power sleep mode when it is not transmitting or receiving data.

The communication module is also preferably equipped with an antenna. The antenna is used to emit the signals produced by the transceiver into the air and also to capture incoming signals for the transceiver to interpret.

The communication module is preferably further equipped with a modulator/demodulator. This component converts digital data into a format suitable for wireless transmission (modulation) and converts received signals back into digital data (demodulation).

The communication module is also preferably equipped with a microcontroller. The microcontroller is a small processor that manages the functions of the communication module, such as initiating connections, managing data transmission and reception, and handling errors.

The communication module is preferably further equipped with memory. The memory stores temporary data during transmission and reception processes. It may also store some configuration details, such as the identifiers of previously connected networks or devices.

The communication module is also preferably equipped with a power management uni. Since wireless communication can consume significant power, the dedicated power management unit serves to minimize power usage and manage power supply to the components of the communication module.

The communication module is also preferably further equipped with interface hardware. This component allows the communication module to interface with the other electronic components of the PBM device, typically through a standard interface such as SPI (Serial Peripheral Interface) or I2C (Inter-Integrated Circuit).

In addition to the foregoing features, and depending on the design of the system, the PBM device may also include a physical user interface. This interface may be as simple as a few LED indicators or buttons, or as complex as a touchscreen interface. The PBM device may further include various safety features. In addition to the safety features managed by the control circuitry, the PBM device may include hardware safety features such as heat sinks to prevent overheating, a fan for cooling, or physical locks to prevent misuse. It will be appreciated that these components may need to be housed in a durable and safe casing, which might also be designed to facilitate the easy application of the device to the user's body.

In some embodiments, the systems and methodologies disclosed herein may be implemented as cloud-based solutions. Utilization of the cloud allows these systems and methodologies to be accessible, scalable, and highly available while ensuring security and compliance with regulatory requirements. Various features of these systems and methodologies may leverage the cloud for various purposes.

For example, the systems and methodologies disclosed herein may use the cloud for data storage and management. In particular, the wellness database, which holds wellness data collected from users, may be stored in suitable cloud storage, such as Amazon S3, Google Cloud Storage, or Azure Blob Storage. This allows for vast, scalable storage, while also allowing the data to be accessed and retrieved from anywhere.

The systems and methodologies disclosed herein may also utilize cloud-based servers. Thus, for example, the server (including the artificial intelligence (AI) engine that accepts treatment objectives and operates on the wellness database) may be implemented as a cloud-based server. This may be a virtual machine or a containerized application running on platforms such as, for example, AWS EC2, Google Cloud Compute Engine, or Azure Virtual Machines. Moreover, some modern cloud platforms offer machine learning and AI services that may be used directly (such as, for example, AWS SageMaker, Google Cloud AI, or Azure Machine Learning), thus simplifying the development and deployment of AI models.

The systems and methodologies disclosed herein may also utilize software clients that are cloud-based. For example, in some embodiments, the software client installed on the client PBM devices may connect to a cloud-based server via the Internet or other networks to send wellness data and to receive PBM recommendations. This client may be an application running on a smartphone, PC or other device associated with a user, or it may run directly on the PBM device if the device has suitable hardware and software support.

The systems and methodologies disclosed herein may also utilize user and administrative interfaces that may be implemented as web-based applications hosted on the cloud. Users and administrators may access these interfaces from anywhere via the Internet or other suitable networks, thus providing flexibility and remote access.

The systems and methodologies disclosed herein may also utilize cloud-based security and compliance solutions. Given the sensitive nature of wellness data, preferred embodiments of the systems and methodologies disclosed herein include security measures such as data encryption at rest and in transit, strong user authentication, access control, and logging and monitoring. Various cloud providers are available which offer services that help meet these needs. For example, AWS offers services such as IAM for access control, CloudTrail for logging, and KMS for key management. Similarly, compliance with regulations like HIPAA may be achieved with specific configurations and services offered by cloud providers.

The systems and methodologies disclosed herein may also leverage the cloud to improve scalability and availability. A significant advantage of implementing the system in the cloud is the ability to scale resources to meet demand. Cloud services are also designed to be highly available, thus ensuring that the system is always operational.

The implementation of the systems and methodologies disclosed herein as cloud-based solutions allows their use in a broad range of applications, some of which were heretofore unavailable. For example, cloud-based implementation of these systems and methodologies facilitates real-time remote monitoring of patients. In particular, medical professionals can observe patients' wellness data and treatment progress remotely, which is particularly beneficial for patients in remote areas or those with mobility issues. Cloud-based wellness systems of the type described herein may also be integrated with existing electronic health record (HER) systems. This integration may provide a more holistic view of the patient's health, potentially improving the effectiveness of PBM treatment recommendations.

The implementation of the systems and methodologies disclosed herein as cloud-based solutions also be integrated with telemedicine platforms or to support telemedicine capabilities. In particular, it allows patients to consult with healthcare professionals virtually via the user interface, thus allowing them to discuss their wellness data, treatment objectives, and PBM recommendations. It also allows healthcare professionals to access a patient's wellness data and treatment objectives, adjust PBM recommendations remotely, and provide personalized advice or counseling based on the data. Healthcare professionals or wellness coaches may also monitor user wellness data and PBM treatment progress remotely in real-time, thus facilitating more timely interventions and adjustments to treatment plans.

The implementation of the systems and methodologies disclosed herein as cloud-based solutions also facilitates research and clinical trials. In particular, with the users' consent, the aggregated and anonymized wellness data collected by the system may be used for research purposes and clinical trials. Hence, such data may be used to study the effectiveness of different PBM parameters and to improve the understanding of the dose-effect relationship of PBM in various conditions. Similarly, such cloud-based systems may also enable collaborative research initiatives. By anonymizing and aggregating user data, these systems may provide rich datasets for researchers studying wellness, PBM, or related areas.

The implementation of the systems and methodologies disclosed herein as cloud-based solutions also facilitates collaboration with other wellness applications or devices. In particular, it provides a means by which data may be gathered from various sources to provide a more comprehensive overview of a user's health and wellness. It also allows for the development of companion mobile applications to enable users to access their wellness data, receive treatment recommendations, and interact with their PBM device on-the-go, from anywhere in the world.

The implementation of the systems and methodologies disclosed herein as cloud-based solutions also facilitates multi-device support. With a cloud-based system, users can interact with the system from multiple devices (such as, for example, smartphones, tablets, or PCs), thus offering convenience and flexibility.

The implementation of the systems and methodologies disclosed herein as cloud-based solutions also offers more insights into personal health and wellness. As a result of the more comprehensive data collection and processing capabilities offered by cloud-based solutions, the systems disclosed herein may provide users with more personalized insights about their health and wellness, including trends, potential risk factors, and tips for improving their wellness outcomes.

The implementation of the systems and methodologies disclosed herein as cloud-based solutions also offers improvements in accessibility. Because they are cloud based, these systems may be accessed globally, thus allowing users to stay connected with their wellness journey regardless of their geographic location. This global reach may also be beneficial for multinational clinical trials or for providing wellness services to a broader population.

The implementation of the systems and methodologies disclosed herein as cloud-based solutions also facilitates large-scale data analytics. With the wellness data being centrally stored on the cloud, advanced big data analytics may be performed on a larger scale. These insights may then be used to further optimize treatment recommendations, identify broader trends in wellness data, or even predict potential health issues based on these trends. Moreover, with access to large datasets of the type that such systems would provide, AI engines may use more complex machine learning models, or use ensemble methods, which may improve prediction accuracy for PBM treatment recommendations.

The implementation of the systems and methodologies disclosed herein as cloud-based solutions also allows them to be integrated with various IoT devices used by individuals in their daily lives. This may include fitness trackers, smartwatches, or other health-monitoring devices. This integrated data could contribute to a richer and more accurate wellness profile for each user.

The implementation of the systems and methodologies disclosed herein as cloud-based solutions also enhances user experience. In particular, cloud-based systems may leverage advanced technologies such as augmented reality (AR) or virtual reality (VR). This may allow users to visualize their wellness data in a 3D format or to engage in VR-based wellness exercises or therapies.

The implementation of the systems and methodologies disclosed herein as cloud-based solutions also allow users to engage in group therapy sessions or support groups. In such applications, users may share their experiences, progress, and encouragement with each other. This may foster a sense of community and provide additional motivation for users.

The implementation of the systems and methodologies disclosed herein as cloud-based solutions also allows companies and organizations to use these systems as part of their corporate wellness programs. In such applications, employees' wellness data may be monitored and appropriate wellness initiatives and recommendations may be made to improve the overall health and productivity of the workforce.

The implementation of the systems and methodologies disclosed herein as cloud-based solutions also supports real-time alerts and notifications to users based on their wellness data. For example, if a user's data indicates a potential health issue, the system could immediately notify the user and recommend immediate action or consultation with a healthcare professional.

The implementation of the systems and methodologies disclosed herein as cloud-based solutions also allows these systems and methodologies to leverage continuous learning algorithms. In particular, as the system collects more data over time, the AI engine can continue to learn and adapt, improving the accuracy and effectiveness of its PBM treatment recommendations.

The implementation of the systems and methodologies disclosed herein as cloud-based solutions also provides multi-lingual support. In particular, because such solutions typically have the ability to support multiple languages, implementation as a cloud-based system may make PBM-based wellness promotion systems accessible to a wider range of users across different geographies and cultures.

The implementation of the systems and methodologies disclosed herein as cloud-based solutions also facilitates their integration with electronic health records (EHRs). In particular, cloud-based implementation allows a user's wellness data and treatment objectives to be securely shared with other healthcare providers, improving the overall continuity and quality of care.

The implementation of the systems and methodologies disclosed herein as cloud-based solutions also facilitates the gamification of wellness. In particular, such cloud-based systems may incorporate gamification elements such as rewards, challenges, and social sharing to motivate users to consistently follow their treatment recommendations and improve their wellness scores.

The implementation of the systems and methodologies disclosed herein as cloud-based solutions also allows for the creation of systems which offer real-time, AI-based wellness advice to users. For example, such a system may suggest adjustments to a user's PBM treatment plans based on their latest wellness data, global trends, or recently published medical research.

As previously noted, virtual reality technology may be incorporated into some embodiments of the wellness promotion systems disclosed herein. Integrating VR technology into these wellness promotion systems may enhance user engagement, improve understanding of the treatment process, and provide a more holistic and enjoyable wellness experience. VR technology may be utilized to create exciting opportunities for improved user experience, adherence, and overall treatment efficacy, while providing a futuristic approach to wellness that makes therapy sessions interactive, immersive, and more engaging.

There are many ways in which VR technology may be incorporated into various embodiments of the systems and methodologies disclosed herein. For example, VR may be utilized to create immersive environments that may enhance the PBM therapy experience. For instance, during a PBM session, users may wear a VR headset that transports them to a calming and tranquil environment such as a beach, forest, or mountaintop. This may help reduce stress and anxiety, which may improve overall wellness.

In some embodiments, VR may be leveraged to allow users to engage with a 3D model of their own body to understand their treatment better. This approach may allow them to visually observe target areas for PBM treatment, see the potential effects of the treatment, and interact with the model to better understand their wellness data.

The system may also provide VR-based educational content related to wellness and PBM therapy. For example, it may show a 3D animation of how light therapy works at the cellular level, which may help users understand and trust the treatment process more.

VR may be utilized to enhance mind-body therapies such as meditation or guided relaxation, which may be offered in tandem with PBM treatments. In such embodiments, the VR environment may guide users through relaxation exercises while the PBM device delivers therapy, thereby creating a more holistic wellness experience.

VR may also be utilized to enhance remote consultations with healthcare providers or wellness coaches. Instead of a simple video call, VR may allow for more interactive and engaging consultations, where the healthcare provider and user can review the user's wellness data in a 3D virtual environment.

The wellness promotion system may also be integrated with VR fitness applications. In such embodiments, the PBM device may be used pre-or post-workout to aid with muscle recovery, improve circulation, or manage pain, tailoring its recommendations based on the intensity or type of VR workout completed or contemplated.

In a VR environment, a personalized virtual assistant may be provided to guide users through their wellness journey. This AI-based assistant may explain the PBM treatment process, answer queries, and even demonstrate correct device usage. By providing interactive and real-time support, the assistant may increase user engagement and adherence to treatment recommendations.

Using VR, real-time visual feedback of treatment progress may be presented. In some embodiments, for example, users may watch as a virtual representation of their body responds to PBM therapy, with inflamed or problem areas gradually changing color or appearance to indicate improvement. This instant visual feedback may be highly motivating and informative.

For users recovering from injuries, VR may offer interactive physiotherapy exercises that coincide with PBM treatment. For example, a user with a knee injury may perform virtual exercises designed to strengthen their knee, while the PBM device provides supportive therapy. The VR environment may monitor and provide feedback on the user's form and progress, making the rehabilitation process more engaging and effective.

VR may also be utilized to gamify the treatment process. In such embodiments, users may earn points, rewards, or progress through levels by adhering to their treatment schedule, achieving wellness goals, or learning more about their wellness data. This may significantly boost user motivation and engagement with their wellness journey.

For group therapies or family wellness programs, multi-user VR sessions may be facilitated. Users may engage in group relaxation, exercise, or educational sessions while simultaneously receiving their individualized PBM treatments.

In cases where users suffer from mental health issues (such as, for example, phobias or PTSD), Virtual Reality Exposure Therapy (VRET) may be incorporated into the system. This technique uses VR technology to expose users to the situations or environments that cause distress in a controlled manner, aiding in their treatment process.

As previously noted, sin some embodiments of the systems and methodologies disclosed herein, VR may be utilized to allow users to engage with a 3D model of their own body to understand their PBM treatment better, and in which they can visually observe the target areas for PBM treatment, see the potential effects of the treatment, and interact with the model to better understand their wellness data. In such a wellness system, the use of VR technology provides an immersive and interactive platform for users to understand and engage with their PBM treatment better. This VR-enabled wellness system provides a comprehensive, user-friendly, and engaging solution for personal health management, promoting adherence to PBM treatment plans and facilitating a more in-depth understanding of personal wellness data.

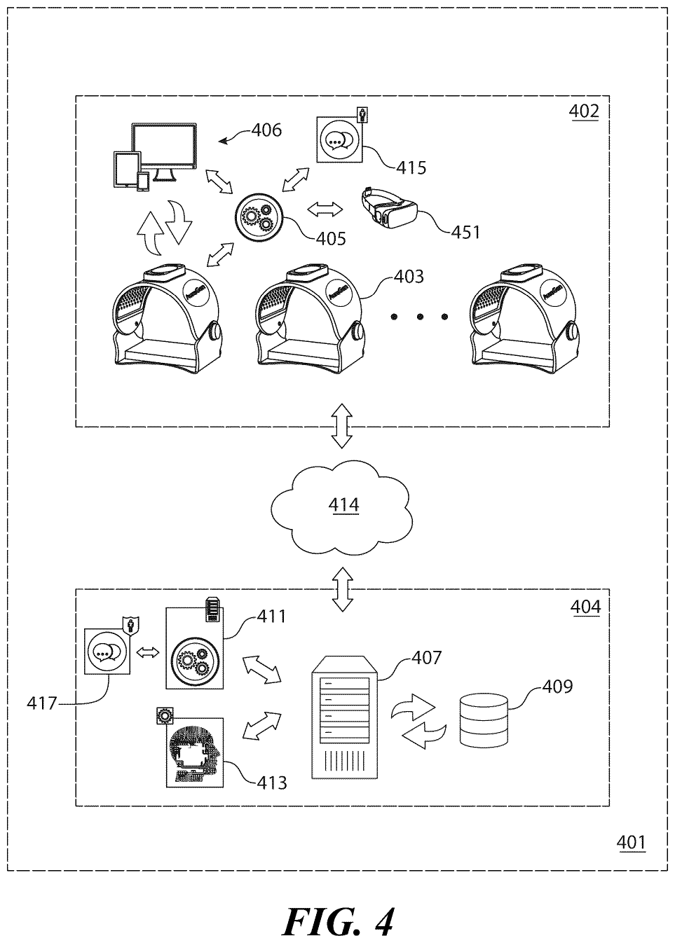

A particular, nonlimiting embodiment of such a system is depicted in FIG. 4 (wherein like numbers depict like elements to the system of FIGS. 1-3). As seen therein, such a system 401 preferably comprises a server 407, a plurality of PBM devices 403, a VR system 451, and optionally a distributed ledger system (not depicted, but stored in a memory system associated with the PBM device 403 or an associated user device 406). Each PBM device 403, associated with a user, communicates wellness data to the server 407, which analyzes this data to generate personalized treatment recommendations.

The VR system 451 provides a 3D model 503 (see FIG. 5) of the user's body, allowing for a visual and interactive understanding of the treatment process. Users can observe target areas 505 for their PBM treatment on the 3D model 503 and see potential effects 507, 509 of the treatment, enhancing their comprehension of the treatment plan. Interactive features may be provided to enable users to manipulate the 3D model 503, inspect particular areas of their body, and visually understand their wellness data.

Furthermore, visual cues may be provided in the VR environment guide users in the correct application of PBM devices, offering clear directional indicators for device placement, and displaying prompts or instructions for treatment. This hands-on approach enables users to actively participate in their treatment process, encouraging better adherence to treatment recommendations.

Additionally, the system 401 incorporates a user interface 415 that allows users to adjust treatment parameters, initiate or terminate a treatment session, or schedule future treatments, facilitating effective management of their wellness regime.

The system 401 may optionally record wellness data and treatment recommendations onto a distributed ledger system. This record-keeping process ensures data security and integrity, with each record being cryptographically secure, time-stamped, and appended to the ledger, creating an immutable, transparent record of the treatment process.

Cloud-based versions of the wellness promotion systems disclosed herein lend themselves to the provision of numerous remote services. These may include, for example, remote monitoring services, where healthcare professionals or wellness coaches monitor user wellness data and PBM treatment progress remotely in real-time. This may facilitate more timely interventions and adjustments to treatment plans. Similarly, such systems may be leverages by telehealth providers to allow users to schedule and engage in remote consultations with healthcare professionals or wellness coaches. The professionals may review the user's wellness data and PBM treatment outcomes beforehand to provide personalized advice and recommendations. In a similar manner, the system itself may be adapted to provide real-time, AI-based wellness advice to users. For example, the system may suggest adjustments to user PBM treatment plans based on their latest wellness data, global trends, or recently published medical research.

Cloud-based versions of the wellness promotion systems disclosed herein also permit remote device management. In such embodiments, technical support staff may remotely manage and troubleshoot the PBM devices and may also push software updates to the devices to enhance their performance or add new features.

Cloud-based versions of the wellness promotion systems disclosed herein may also be leveraged to provide virtual community support. In such embodiments, users may participate in virtual community support groups with other users who have similar wellness goals or challenges. This may enhance their motivation and engagement with the PBM treatment. At the same time, the system may provide personalized learning resources. Thus, for example, the system may recommend personalized learning resources based on the user's wellness data and PBM treatment outcomes. These may include articles, videos, or online courses about wellness topics that are relevant to the user. Systems of this type may use machine learning to identify patterns and predict potential health issues before they arise, notifying users and their healthcare providers to take preemptive action.

Cloud-based versions of the wellness promotion systems disclosed herein also facilitate the provision of comprehensive data reporting services. Thus, for example, medical professionals and other such parties may use such a system to generate detailed reports about a client's progress, compare the effectiveness of different PBM treatment plans, or provide insights into wellness trends among their client base. Similarly, systems of this type may be integrated with other health services such as online pharmacies, nutrition counseling, or fitness coaching. This may provide a holistic, one-stop solution for users seeking to enhance their wellness.

The ability of PBM devices to communicate with each other in the systems and methodologies described herein may also facilitate new avenues of PBM consumption, while further enhancing the user experience and efficacy of wellness promotion systems centered around the PBM devices. It creates possibilities may make the wellness promotion system more effective, personalized, and dynamic, ultimately leading to better wellness outcomes for users.

For example, the ability of PBM devices to communicate with each other allows the PBM devices to collectively learn from the wellness data and treatment outcomes of all users, not just the individual user. By sharing anonymized data, the PBM devices may facilitate a more robust and efficient learning process for the AI engine, improving treatment recommendations for all users. In households with multiple users and PBM devices, it also allows treatments to be synchronized across devices. This may be especially beneficial for treatments that might affect the overall environment, such as light therapy that affects circadian rhythms.

Moreover, with device intercommunication, group wellness programs or challenges may be initiated. For example, families, friends, or coworkers may participate in a wellness program together, sharing progress and achievements to motivate each other. Users may share their treatment plans, progress, and achievements via their devices to foster a social wellness community, which may motivate users and make the treatment process more enjoyable. Moreover, the PBM devices may share information about the effectiveness of treatments based on the users' locations, thus allowing the system to provide location-specific wellness recommendations. The PBM devices may also communicate to synchronize healing sessions among users who wish to partake in shared PBM treatments. This could be particularly useful in a communal living or therapeutic environment, fostering a sense of unity and shared wellness goals.

In multi-user environments such as nursing homes or rehabilitation centers, such inter-device communication allows PBM devices to be used to coordinate care for multiple patients. For example, if one patient's device registers a significant change in their wellness data, it may communicate this to other devices in the vicinity to adjust their treatment schedules or parameters if needed. Similarly, if several users in the same environment are using PBM devices, inter-device communication may enable the creation of an adaptive ambient environment. For example, the PBM devices may cooperate to collectively adjust the level of ambient light in a room based on the collective wellness data and treatment objectives of all present users. The devices may also cross-check the data they collect from different users in a similar environment to verify the accuracy of readings, thus enhancing the reliability of wellness data being collected and analyzed. In addition, if the devices have the ability to communicate with each other, they can potentially share information about the effectiveness of treatments based on the users' locations, thus allowing the system to provide location-specific wellness recommendations. In professional settings such as clinics or wellness centers, PBM devices may share data and treatment outcomes for the betterment of overall client service, and professionals may use this shared knowledge to make informed decisions regarding client treatment.

Inter-device communication may also be adopted to ensure device redundancy. In particular, in the event that one device fails or runs out of battery, another nearby device may take over its functions temporarily, ensuring that the user's wellness data continues to be collected and that they continue to receive their PBM treatment recommendations. Similarly, in a multi-device environment, tasks such as data processing and analysis may be distributed among multiple devices. This may reduce the workload of the server and improve system efficiency.

Various embodiments of the systems and methodologies disclosed herein are possible in which two or more PBM devices disposed in disparate locations (for example, home and work, or home and at a healthcare facility) may be utilized to provide therapy to a single user in a coordinated fashion. By intelligently coordinating PBM devices in multiple locations, the system can offer a personalized, adaptable, and highly responsive approach to wellness promotion.

For example, such a system may allow a user to set different locations (such as, for example, home, work, gym), each with its PBM device. The AI engine could then design a treatment plan that adapts to the user's daily routine. For instance, the system may schedule a morning treatment session on the home device, an afternoon session at work, and a post-workout treatment at the gym. These schedules may also be adapted in real-time based on the user's location data.

The system may store user profiles on the cloud, allowing any PBM device associated with the user to access the latest treatment protocols, user feedback, and wellness data. Consequently, for example, a device at work could pick up where the home device left off, ensuring a consistent therapy experience throughout the day.

Such a system may also track and communicate the progress of treatments between devices. For example, if a user had to leave home mid-treatment, the work device could complete the remaining session once the user arrives at work. This may ensure that the user receives the full benefits of each treatment session, even when moving between locations.

Such a system may also enable remote adjustment of treatment plans. If a user feels the need to modify their treatment objectives or schedule, they may make these changes on one device, and the updated plan would be instantly available to all other devices.

In such a system, data from all devices, regardless of location, may be combined to provide a comprehensive overview of the user's wellness and treatment effectiveness. This may help the AI engine make more accurate PBM recommendations and adjust the treatment plan based on the user's changing needs and environment.

Such a system may also send reminders or notifications to the user, prompting them to continue their therapy when they reach a location with an associated PBM device. These reminders may help the user maintain consistency in their treatment.

Such systems may also calibrate the intensity and duration of PBM therapy based on the cumulative output from all devices a user interacts with throughout the day. If a user has received a portion of their daily recommended therapy at home, the device at their workplace may adjust accordingly to ensure optimal total dosage or treatment at different wavelengths.

Different environments can impact wellness factors. For instance, a stressful workplace might increase a user's heart rate or blood pressure. PBM devices in these locations may be programmed to respond to these unique environmental stressors with targeted therapy protocols, helping to restore equilibrium and promote wellness.

If the user has specific wellness goals that involve multiple body parts (such as, for example, full-body recovery after intense physical activities), the system may design a treatment plan that coordinates multiple devices. For example, a device at home might focus on upper body recovery, while the device at work might cater to the lower body.

In a system incorporating wearable PBM devices, fixed location devices may supplement treatment. For example, a wearable device might provide ongoing low-level treatment throughout the day, while fixed devices at home or work deliver more intense therapy sessions at scheduled times.

By sharing real-time wellness data across devices, the system can also provide dynamic treatment adjustments. For instance, if a user's stress levels peak at work (as indicated, for example, by blood pressure or other relevant data), the home device may be ready to offer a calming PBM treatment as soon as they arrive home.

In a broader ecosystem of users, the system may enable location-based group challenges or leaderboards, encouraging users to engage with their PBM devices regularly and foster a sense of community around wellness.

In such systems, each PBM device may also learn from the user's interaction with other devices and apply these insights to its own treatment protocols. For example, if the user often increases the light intensity at their home device, the work device might suggest a similar intensity increase for their next session.

For certain wellness objectives, it may be beneficial to sequence treatments across different devices. As a particular, nonlimiting example, a user could start their day with a stimulating therapy at home, have a maintenance session at work, and end with a relaxing treatment at their home device.

If the user interacts with other health devices (such as, for example, fitness trackers or smart scales), each PBM device may receive this data and adjust treatments accordingly. For example, after an intense workout, a PBM device at work may offer a recovery treatment tailored to the user's exercise type and intensity.

PBM devices at home and work may also adapt treatment protocols to a user's circadian rhythms. For instance, morning treatments at home may be designed to stimulate wakefulness, while evening treatments focus on relaxation and recovery. Similarly, such systems may offer distinct wellness programs for home and work, each designed to counteract the specific stressors of these environments. For example, s work program might focus on posture and eye strain, while a home program may prioritize sleep quality and relaxation.

Such systems may also consolidate user feedback from all devices to refine the AI model's understanding of the user's preferences and treatment responses. This consolidated data may help to improve the system's treatment recommendations.

The system may also enable different privacy settings for each device. For example, a user may allow more personal data to be collected and used by their home device compared to their work device.

In addition to embodiments where two or more PBM devices associated with a user are disposed at different locations and communicate with each other, embodiments of the systems and methodologies disclosed herein are also possible where two different PBM devices that are co-located communicate with each other in a similar manner to provide the same or different benefits. For example, in some embodiments, wellness promotion systems may utilize two or more PBM devices to provide more comprehensive therapy, treating multiple body parts simultaneously or sequentially. This may be especially beneficial for users who have multiple target areas for treatment or for those who require a full-body wellness approach.

In some embodiments of this type, all of the PBM devices may be synchronized to provide light therapy in a coordinated manner. For example, if a user has arthritis in both knees and elbows, PBM devices may be positioned on these areas and programmed to deliver identical treatments at the same time. The devices may communicate with each other via suitable protocols (such as, for example, Bluetooth or Wi-Fi) to ensure the therapy is synchronized and uniform.

For conditions that may benefit from a sequential light therapy approach, one device may initiate the treatment, with the others following in a predetermined order. For instance, if a user requires a specific sequence of therapy (for example, first lower back, then shoulders), the devices may communicate with each other to follow this order, starting and stopping as required.

Each PBM device may also be programmed to deliver different treatments based on the needs of the body part it's associated with. For example, a device on the lower back may offer a different intensity, duration or wavelength profile of light therapy compared to a device placed on the neck. The server or AI engine may analyze wellness data for each body part and generate tailored PBM recommendations. The devices may then exchange information about their respective treatments to optimize the overall therapy protocol, avoiding potential conflicts or detrimental overlaps in therapy.

The PBM devices may also be designed to adapt their treatment protocols based on feedback from each other. For instance, if one device detects a substantial improvement or an adverse reaction in its treatment area, it may signal the other devices to adjust their treatments accordingly. This kind of inter-device communication may make treatments more responsive and personalized. Of course, it will be appreciate that suitable AI learning as described herein may be leveraged to assist in this process.

For users with comprehensive wellness goals or systemic conditions, multiple PBM devices could be deployed across the body as part of a full-body treatment regimen. These devices could be coordinated to provide a holistic therapy session, communicating with each other to ensure an optimized, full-body treatment protocol.

In some embodiments, each device may be programmed with a specific therapy schedule and synchronized with other devices for a harmonized treatment experience. The scheduling may consider factors such as the severity of symptoms in different body areas, optimal treatment times, and user availability. This level of coordination may help avoid overexposure to light therapy and ensure that each body part (and the body as a whole) receives optimal care.

In some embodiments, PBM devices may be designed to adapt their treatments in real-time based on feedback from other devices. For instance, if a device treating the user's lower back reports heightened sensitivity or discomfort, devices working on adjacent areas (for example, hips or thighs) may immediately adjust their treatment intensity or switch to a more soothing protocol.

The system may also include an integrated user feedback system, allowing users to report their real-time comfort level, perceived effectiveness, or any discomfort during the therapy. This feedback may be shared amongst the devices, allowing them to adjust their operation dynamically for maximum user comfort and therapeutic benefit.

If a user has undergone an intense exercise session or has a particular area recovering from an injury, the system may activate a “recovery mode”. In this mode, one or more PBM devices may focus on the affected areas, while the rest of the devices may continue with their regular treatment schedule or adjust to a less intense therapy.

In a holistic treatment plan, the system may be designed to work in tandem with other therapeutic methods. For example, a PBM device may be programmed to deliver light therapy in conjunction with physical therapy sessions or post-medication schedules. Other devices may adjust their protocols based on the primary treatment, ensuring a complementary and enhanced therapeutic effect.

In a household with multiple users, each user may have specific PBM device(s) associated with their unique wellness profiles. The system may manage the operation of all of these devices, ensuring personalized therapy for each user while maintaining a coordinated schedule to avoid device conflict or interference.

While PBM devices of the type depicted in FIGS. 4-6 may be utilized in many embodiments of the systems and methodologies disclosed herein, some embodiments may also utilize wearable PBM devices. Such wearable PBM devices may be designed to wirelessly communicate with the wellness promotion system, transmitting user data and receiving personalized treatment recommendations.

Possible wearable PBM devices include, for example, PBM headbands or caps that may be designed to treat conditions such as migraines, insomnia, and mood disorders, or to stimulate hair growth. Devices of this type may be adapted to deliver light therapy to the scalp and the frontal cortex of the brain, and they are typically easy to wear and adjust.

Possible wearable PBM devices also include PBM eye masks, glasses or goggles. Such devices can be utilized for treating conditions related to eye health, such as macular degeneration, or for general wellness purposes like promoting better sleep.

Possible wearable PBM devices also include PBM neck bands. These devices may deliver PBM therapy to the neck region, which can help in treating conditions such as thyroid disorders, neck pain, and stress, or may be used as adjuvants to medical procedures such as carotid endarterectomies.

Possible wearable PBM devices also include PBM wraps or belts. These are versatile devices that may be wrapped around different body parts such as, for example, the waist, knee, elbow, or ankle. They may be utilized to alleviate pain, reduce inflammation, and promote healing in those specific areas.

Possible wearable PBM devices also include PBM gloves or socks. Such devices may be useful for delivering PBM therapy to the hands and feet, which may be beneficial for conditions such as, for example, arthritis, carpal tunnel syndrome, or peripheral neuropathy.

Possible wearable PBM devices also include PBM posture correctors. These devices may provide PBM therapy while also encouraging proper posture, which may be beneficial for treating back pain and related issues.

Possible wearable PBM devices also include PBM wearable patches. These small, flexible patches may deliver PBM therapy to specific areas of the body. They are portable and easy to use, making them suitable for treating a variety of localized conditions.

Possible wearable PBM devices also include PBM wristbands or watches. Similar to smartwatches or fitness bands, these wearable devices may be designed to deliver PBM therapy to the wrist and hand. This may be especially beneficial for treating conditions such as carpal tunnel syndrome or for promoting better sleep through irradiation of the wrist's acupuncture points.

Possible wearable PBM devices also include PBM shoulder pads. These wearable devices may be designed to sit on the user's shoulders and deliver PBM therapy to the shoulder and upper back region. Devices of this type may be especially useful for treating conditions such as frozen shoulder or general muscle tension.

Possible wearable PBM devices also include PBM lumbar belts. These belts may deliver PBM to the lower back, aiding in pain relief, reducing inflammation, and promoting healing of the lumbar region, which can often be affected by conditions such as sciatica or general lower back pain.

Possible wearable PBM devices also include PBM facial masks. PBM facial masks may provide light therapy to the entire face, which may aid in skincare (such as, for example, the treatment of acne, wrinkles, skin rejuvenation), relieve sinus pressure, or even help with mood disorders through irradiation of the facial acupuncture points.

Possible wearable PBM devices also include PBM earbuds or headphones. These devices may deliver PBM therapy into the car canal or around the auricular area, and may be beneficial in treating conditions such as tinnitus or for stimulating the vagus nerve, which has wide-reaching effects on overall wellbeing.

Possible wearable PBM devices also include PBM vests or jackets. A wearable vest or jacket incorporating PBM technology may provide broad coverage for the chest, back, and possibly the arms, which may aid in the treatment of multiple conditions or in general wellness promotion.

Possible wearable PBM devices also include PBM insoles. These devices may be placed in shoes to deliver PBM therapy to the feet, potentially beneficial for conditions such as plantar fasciitis or peripheral neuropathy.

Various cloud-based wellness systems may be created in accordance with the teachings herein based on PBM devices, especially when there is an associated community of users. In some embodiments, such systems may be utilized to create a network of PBM devices, where each device can communicate with the others. This may enable coordinated treatment schedules for a single user with multiple devices, or even coordination between devices used by different users.

Some embodiments of these systems may utilize a decentralized and distributed database of transactions that may be adapted for the PBM devices. In such embodiments, each device may store the wellness data of its user, and this data may be shared among other devices in the network. The decentralized nature of the wellness data may enhance the system's robustness against data loss.

Some embodiments of such systems may feature an RF collector that aggregates data from all of the PBM devices, normalizes it, and provides insights on a unified platform. Such an RF collector may help health professionals and data scientists to analyze the data more effectively and develop personalized treatment plans.

Communications among different PBM devices in such a system may be established using different protocols based on the system requirements and the capabilities of each device. This may provide flexibility and expand the range of devices that could be integrated into the system. The implementation of a hyper-distribution communications protocol in such systems may allow efficient data exchange among the PBM devices and the cloud server, thus ensuring that each PBM device is updated with the most recent wellness data and treatment recommendations and allowing for real-time updates and adjustments to treatment plans. The use of a distributed system and a hyper distribution communication protocol may have the effect of enhancing data security. In particular, in such a system, each PBM device may validate the transactions (data) independently, ensuring the accuracy and reliability of the wellness data.

As previously noted, distributed ledger-based system may be utilized in some embodiments of the systems and methodologies disclosed herein which feature a cloud-based wellness system that includes a plurality of PBM devices that are in communication with each other. The use of such a system provides various features and advantages. By integrating a distributed ledger-based system into a cloud-based wellness system, these features and advantages may lead to a more secure, efficient, and user-centric approach to wellness and PBM treatment delivery.

One benefit of the use of a distributed ledger is that it provides strong data integrity and security. In the context of a wellness system, all health data and treatment records may be stored on the blockchain. Since blockchains are typically immutable and tamper-evident, this helps to ensure that the records remain unaltered and safe, offering a higher level of data security. The blockchain may provide a transparent, chronological record of all user health data and treatments. This transparency may enable doctors and patients to accurately track progress over time and may be instrumental in identifying the effectiveness of various treatments.

Blockchain technology often includes the use of smart contracts, which are self-executing contracts with the terms directly written into the code. In a wellness system, these may be used to automatically implement personalized treatment plans. For example, if certain health data parameters are met, the smart contract may automatically adjust the treatment regimen on the PBM device.

The use of a blockchain also allows secure and verified communication to be established between different PBM devices. In such a system, each device may share wellness data, treatment plans, and updates securely, ensuring that every PBM device associated with a user stays in sync, irrespective of their location. Moreover, with a decentralized approach, users may have more control over their data and who has access to it and may have the ability to grant or revoke access or use permission to doctors, researchers, or other parties at any time. In some embodiments, an anonymized and secure dataset from the blockchain may be used for research purposes to understand the effectiveness of various PBM treatments on different conditions and across different demographics.

The use of a distributed ledger system also allows for tracking of the transactions of multiple PBM devices. This helps to ensure synchronized and harmonized wellness interventions, even when devices are not in physical proximity to one another.

It will be appreciated that the systems and methodologies disclosed herein lend themselves to a subscription-based web service, which may open up numerous possibilities and conveniences for users. By transitioning to a subscription-based model, the PBM wellness system may provide personalized wellness support to a larger audience while still maintaining a high level of individual customization and control.

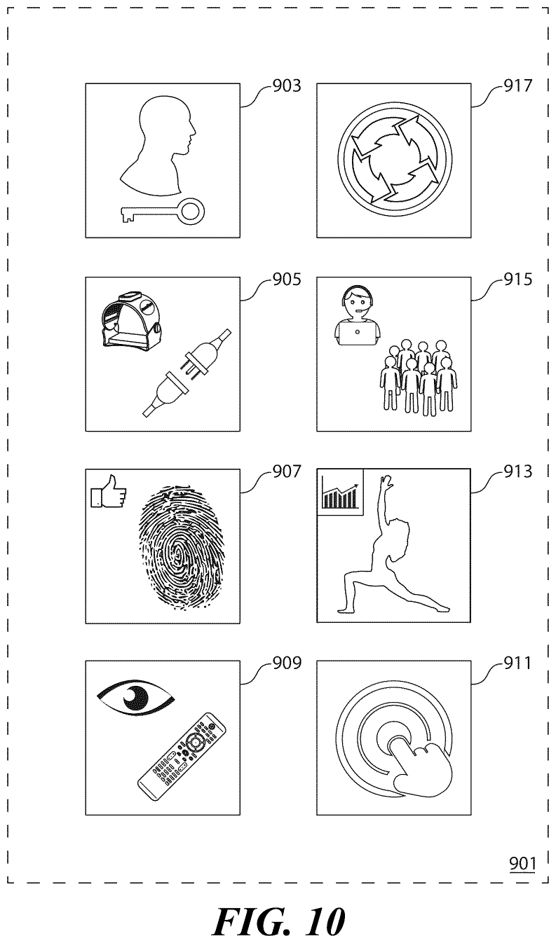

FIG. 10 depicts a particular, non-limiting embodiment of a subscription-based web service in accordance with the teachings herein. The system 901 depicted therein consists of a user registration and profile setup module 903, a PBM device connection module 905, a personalized recommendations module 907, a remote control and monitoring module 909, an interactive features module 911, a wellness tracking and reporting module 913, a community and support module 915, and an updates module 917. Each of these modules is described in greater detail below.

The user registration and profile setup module 903 provides a means for users to sign up for the service through an associated website and set up their personal profile, providing necessary wellness data such as their health history, lifestyle habits, current medical conditions, and treatment objectives. Users may be offered different subscription tiers depending on the level of personalization and features they require.

The user registration and profile setup module 903 initiates a process which involves user registration, profile setup (which includes gathering from the user personal details, medical history, lifestyle habits, current conditions and treatment objectives), privacy and consent, AI analysis and PBM device association. Through this detailed user registration and profile setup process, the PBM wellness system gathers the information required to ensure that each user receives personalized, data-driven treatment recommendations that align with their unique health needs and wellness objectives. The steps of this process are described in greater detail below.

The user registration process will involve creating a unique user account on the website. This will typically require the user to provide an email address (or alternatively a phone number) and to create a secure password. The email address serves as a unique identifier for the user. The system sends a verification link or code to the provided email address to confirm its validity. Once the email address is verified, the account is successfully registered.

Profile setup typically involves collecting crucial wellness data about the user. This may include, but is not limited to, personal details (such as, for example, basic information such as age, gender, height, weight, and physical activity levels); medical history (such as, for example, information about past illnesses, surgeries, chronic conditions, or any medications the user is currently taking); lifestyle habits (such as, for example, information about the user's diet, sleep patterns, exercise routines, and stress levels, which may be used to understand the user's overall wellness state beyond the physical aspect); current conditions (such as, for example, whether users are experiencing any specific ailments, pain points, or areas of discomfort that they want to address through the PBM treatment); and treatment objectives (specific objectives for the PBM treatment such as, for example, pain relief, skin rejuvenation, injury recovery, or stress reduction).

In particular, given that sensitive health data is being collected, it is important for the website to clearly communicate its privacy policy and seek explicit consent from users for data collection and usage. This is handled by the privacy and consent process.

Once the user's profile is complete, this data would be processed by the system's AI engine. In the subsequent AI analysis, the AI engine analyzes the provided wellness data and generates initial personalized treatment recommendations for the PBM devices.

PBM device association is the formal process by which PBM devices are connected to the system. In particular, users are guided to connect their PBM device(s) to the system during the profile setup. The system provides step-by-step instructions on how to link the device (e.g., through Wi-Fi or Bluetooth). Once connected, the device is associated with the user's profile, and starts to receive treatment recommendations from the server based on the user's wellness data.

The PBM device connection process ensures a secure and efficient link between each user's PBM device and the server, enabling personalized and adaptive PBM treatments. In a particular, nonlimiting embodiment, this process involves the steps of device identification, device communication, device verification, device registration, treatment recommendation delivery, and continuous synchronization. These steps are described in greater detail below.

In the device identification step, after successful user registration and profile setup, the user is guided to connect their PBM device to the system. Each PBM device is equipped with a unique identification number or QR code. The user enters this number or scans the QR code using the website interface or a companion mobile application to identify the specific device they wish to connect.

The PBM device is preferably designed to communicate with the server either via an Internet connection using Wi-Fi or via a local connection using Bluetooth. The connection method could depend on the model of the device and the user's preferences. In the device communication step, the user is guided to enable the appropriate settings on their device to establish this communication link. This may involve, for example, entering the Wi-Fi network details on the device or pairing the device with the user's computer or smartphone via Bluetooth.

Once the device is identified and the communication link is established, the server verifies the device in the device verification step. This may involve, for example, checking the device identification number against a database of valid devices. The server may also check the device's firmware version and prompt the user to update the firmware if necessary.

After successful verification, the server registers the device to the user's account in the device registration step. This will typically involve storing the device identification number and any relevant device information (such as, for example, the firmware version or device model) in the user's profile on the server. The server also communicates back to the device to confirm successful registration.

Once the device is registered, the server commences treatment recommendation delivery. This involves the delivery of treatment recommendations to the device based on the user's profile. The server will typically send these recommendations as data packets over an established communication link. The device is equipped with suitable hardware and software to interpret these data packets and adjust its treatment parameters accordingly.

After initial setup, the device preferably synchronizes continuously with the server. This continuous synchronization process may involve sending wellness data collected during treatments back to the server and receiving updated treatment recommendations from the server. This synchronization process ensures that the user's treatment is continuously tailored to their changing wellness needs.

The personalized recommendations process ensures that each user receives a unique and tailored wellness program that aligns with their health needs, lifestyle, and wellness goals, making the PBM therapy more effective and user-centric. In a particular, nonlimiting embodiment, it involves the steps of data collection, data analysis and interpretation, the generation of personalized recommendations, the delivery of those personalized recommendations, and a feedback loop. Each of these features is described in greater detail below.

The system first performs a data collection process, where it gathers and aggregates wellness data from multiple sources. This includes the initial user profile created during the registration process, real-time health and wellness data collected from the PBM device, and any additional wellness data the user provides through the website or app interface. The user's goals, preferences, and any changes in their health or wellness status may be updated in real-time.

Next, the AI engine analyzes and interprets the collected data. In this data analysis and interpretation step, advanced machine learning algorithms may be utilized to identify patterns and trends, predict potential health risks, and determine the most effective treatments based on a combination of data from the user's profile and PBM device.

In the step of generating personalized recommendations, the AI engine generates personalized treatment recommendations for the user based on the data analysis results. These recommendations may include specific light therapy settings (such as, for example, intensity, duration, and frequency), as well as other wellness recommendations (such as, for example, lifestyle changes, exercise, or diet tips). The system may also recommend specific programs or routines available in the PBM device that align with the user's wellness goals.

Once the recommendations are generated, they are delivered to the user. Delivery of the recommendations typically involves sending these recommendations back to the user's PBM device via a secure communication link. These recommendations are then be displayed on the device interface. Alternatively or in addition, if the device is connected to a smartphone or computer, the user may receive these recommendations through the website interface or a companion app.

As the user starts to implement the provided recommendations, the PBM device continuously collects and sends back the user's wellness data to the server. The AI engine then adjusts future recommendations based on the user's progress and any changes in their wellness data. This feedback loop ensures that the treatment recommendations are continuously tailored to the user's evolving wellness needs.

The remote control and monitoring process may be effectively implemented using secure cloud technologies, Internet of Things (IoT) protocols, and suitable real-time data streaming technologies. Implementing remote control and monitoring in this way provides users with greater control over their wellness treatments, allows for real-time adjustments, and improves user engagement with the system. Additionally, this feature enables healthcare professionals to monitor patient wellness remotely and make timely interventions when necessary, potentially improving health outcomes. In a particular, nonlimiting embodiment, the remote control and monitoring process includes the steps or features of device connection, data transfer, server processing, remote control, monitoring and alerts, and security and privacy. Each of these steps of features is described in greater detail below.

For the system to function remotely, each PBM device should be connected to the Internet or another suitable network, either directly or through a user's smartphone or computer. This device connection may be implemented using Wi-Fi, Bluetooth, or other suitable IoT protocols. The device's software client manages this connection, ensuring that data flows securely and smoothly between the PBM device and the server.

Once a connection is established, the PBM device begins data transfer, which includes collecting and transmitting user wellness data to the server in real-time. This data may include device usage patterns, treatment results, and any user feedback or adjustments made during the treatment.

In the server processing step, the server receives this real-time data and processes it, updating the user's profile and making adjustments to treatment recommendations as necessary. The server also manages the distributed ledger system (if one is utilized), adding new records and ensuring that all data is securely stored and time-stamped.

Users may remotely control their PBM devices via the website or companion app. Through this remote control feature, users can start or stop treatments, adjust device settings, choose different treatment programs, and schedule future treatments. These commands are sent to the PBM device via a suitable connection to the Internet or other network.

In the monitoring and alerts feature, the system continuously monitors the device status and user wellness data. If any anomalies or issues are detected, the system sends alerts to the user or, if sufficient permission has been granted, to healthcare professionals or other third parties. Users may also monitor their wellness progress through visual graphs, charts, and wellness reports available on the website or app.

The security and privacy feature ensures that all communication between the PBM devices, the server, and the user interface are encrypted to ensure the privacy and security of user data.

The interactive features module of the PBM wellness system's website subscription service enhances user engagement, provides personalized experiences, and facilitates better wellness outcomes. By creating an interactive environment, the system may help users feel more engaged and in control of their wellness journey. This may ultimately lead to better adherence to treatment plans and better wellness outcomes. In a particular, non-limiting embodiment, this module implements the steps or features of a user dashboard, a 3D body model, a community forum, educational resources, customer support, and feedback/reviews. Each of these steps or features is described in greater detail below.

The website features an interactive user dashboard. Here, users may track their treatment progress, check wellness statistics, schedule treatments, and manage their devices. The dashboard may include interactive charts, graphs, and other visual aids to help users understand their wellness data. It may also include features such as, for example, reminders for scheduled treatments, alerts for device issues, and notifications for updates or new features.

In order to provide a more immersive understanding of the PBM treatment, the website may integrate a 3D body model feature. Users may interact with this model to visually understand the target areas for treatment, the potential effects, and the progress over time. Advanced 3D modeling and VR technologies may be employed for this feature to provide a realistic and interactive experience.

The website may host a community forum where users can interact with each other, share their experiences, ask questions, and provide support. The forum may be moderated to ensure the discussions are relevant, respectful, and comply with privacy regulations. This feature may enhance user engagement and provide valuable user feedback.

The website may feature a section dedicated to educational resources such as, for example, articles, blogs, videos, FAQs about PBM treatment, wellness tips, and information about how to make the most of the PBM devices. These resources may be interactive, and may be equipped with features such as quizzes, surveys, and comment sections.

A live chat feature may be integrated into the website for real-time customer support. Users may reach out for technical help, inquiries about the service, or any other issues. Chatbots powered by AI may be utilized to handle common queries, while more complex issues may be routed to human support staff.

The website may also feature a system for users to provide feedback and reviews. This may include, for example, user experience ratings with the PBM devices or feedback on the effectiveness of treatments and overall satisfaction with the service. This may provide valuable insight for improving the system and services.

The wellness tracking and reporting module of the PBM wellness system's website subscription service provides users with a detailed, personalized, and actionable understanding of their wellness journey, empowering them to take active control of their health and wellness. In a particular, non-limiting embodiment, this module provides the features or steps of wellness data collection, data transmission and storage, data analysis, personalized reporting, alerts/notifications, and data sharing. Each of these features or steps is described in greater detail below.

Each PBM device connected to the system implements regular wellness data collection in which it collects wellness data from its associated user. This data may include metrics related to the PBM treatment itself (such as, for example, duration, frequency, and light wavelength used), physiological responses to the treatment (such as, for example, changes in heart rate, blood pressure, or skin temperature), and subjective user feedback (such as, for example, use feedback relating to pain levels, mood, or sleep quality).

The data transmission and storage feature is then leveraged to transmit the wellness data securely from the PBM device to the cloud server, either directly or via the user's connected smartphone or computer. The wellness data may be time-stamped and stored in the server's database, thereby maintaining a comprehensive and chronological record for each user.

The wellness data is then processed and analyzed by the system's AI engine to derive meaningful insights and patterns. This data analysis may involve, for example, identifying correlations between treatment parameters and wellness outcomes, tracking progress over time, or predicting future wellness trends based on historical data.

The analyzed data is then presented to the user in an easy-to-understand format via the website's user dashboard. This personalized reporting may include visual elements such as, for example, graphs, charts, and heat maps, and might cover aspects such as, for example, treatment adherence, progress towards wellness goals, comparison of different treatment periods, or the effectiveness of different treatment parameters.

The system also generates alerts and notifications based on the analyzed data. For example, it may remind users to start their scheduled PBM treatment, warn them if their wellness parameters deviate from normal ranges, or congratulate them when they reach a wellness milestone.

With user consent, the wellness data and reports may be shared with healthcare providers, wellness coaches, insurance providers, or other third parties. Such data sharing may facilitate remote monitoring and personalized guidance. The data may be anonymized and aggregated for research purposes, contributing to the broader scientific understanding of PBM therapy.

The community support feature of the PBM wellness system's website subscription service is preferably designed to foster a supportive, engaging, and motivating environment for its users. By implementing these features, the Community Support system may provide an inclusive, engaging, and supportive platform for users to interact with each other, learn, and share their wellness journeys, making the overall wellness experience more enjoyable and sustainable. This feature typically includes the features of user forums, wellness challenges or leaderboards, social media integration, virtual support groups, peer connection, user testimonials and case studies, and expert participation. Each of these features is described in greater detail below.

User forums or discussion boards may be a key component of community support. These forums or boards provide a platform for users to post questions, share their experiences and results, provide support and encouragement, and exchange advice related to PBM treatment and wellness. These forums or boards will preferably be moderated to ensure a safe and respectful environment.

The system preferably features wellness challenges or leaderboards. In particular, the system may host wellness challenges, such as committing to a given frequency of PBM treatments or achieving specific wellness goals. Leaderboards may be utilized to display users who are leading or have completed these challenges, fostering a sense of healthy competition and camaraderie.

Users may have the option to connect their profiles to their social media accounts, enabling them to share their wellness journey, achievements, and participation in challenges with their wider social network. Such social media integration not only promotes community support but also raises awareness and acceptance of PBM therapy.

Depending on the demographics and needs of the users, the system may facilitate virtual support groups. These groups may focus on specific topics or user groups, such as managing chronic pain with PBM, PBM for athletes, or new users starting their PBM journey. These groups may be facilitated through video meetings or live chat sessions.

Peer connection features could be implemented to allow users to follow, message, or otherwise connect with each other, subject to privacy settings. This allows for the formation of friendships, mentorships, and a sense of belonging within the community.

The system preferably leverages user testimonials and case studies to serve as motivation and provide real-world examples of what can be achieved with regular use of the PBM treatment and the wellness system.

The system also preferably leverages expert participation. The presence of experts, such as healthcare providers or wellness coaches, within the community may add value by providing accurate information, answering queries, and offering professional advice. This may be facilitated through scheduled “Ask Me Anything” sessions, webinars, or guest blog posts.

The continuous updates module of the PBM wellness system's website subscription service acts to ensuring the platform stays current, secure, and user-friendly by providing timely updates. These updates may be thoroughly tested before being rolled out to the live system to avoid any disruption of service. The system may also incorporate user prompts or notifications to inform users of any important updates or changes. By implementing a rigorous and user-focused continuous update strategy, the system may be able to provide a robust, secure, and effective wellness platform. In a particular, nonlimiting embodiment, the continuous updating module includes the features of software and firmware updates, security updates, feature updates, algorithm improvements, content updates, user interface and experience updates, database updates, and regulatory compliance updates. Each of these features is described in greater detail below.

Software and firmware updates to the server software, user application, and device firmware ensure that the system stays updated with the latest features and improvements. These updates may be rolled out automatically or manually, based on user settings. Firmware updates may be pushed directly to the PBM devices via a suitable network or Internet connection, ensuring that they have the latest functionalities and security patches.

Security is paramount in a system that handles sensitive health data. Regular security updates may include the latest encryption technologies, patches for any identified vulnerabilities, and improvements in user authentication methods.

Continuous innovation may be a key to the platform's success. User feedback, advancements in wellness practices, and evolving technology trends may guide the development of new features. These feature updates may be integrated into the platform through regular updates.

As more data is collected from the users, the system's recommendation algorithms may be continually refined and improved to provide better, more personalized recommendations. These algorithm improvements may be integrated into the system as updates.

Continuous updates may also involve regularly updating the system's content. Such content updates may include new wellness articles, videos, tutorials, user guides, and other educational or informational content.

To ensure a smooth and user-friendly experience, regular updates to the platform's user interface and user experience design may be carried out. These user interface and experience updates may be based, for example, on user feedback and industry best practices.

Updates may also be made to the underlying database structures to ensure the efficient handling and storage of user data. Such database updates may involve updates to data schemas, storage techniques, or database management systems.

With changing healthcare and data privacy regulations across different regions, the system may need to implement regulatory compliance updates regularly in order to stay in compliance with these changes.

The subscription based services described herein may be monetized in various ways. These include, without limitation, through the use of subscription fees, device sales, partner collaborations, paid features, advertising, data monetization, and corporate or institutional wellness programs. These monetization features are described in greater detail below. It will be appreciated that the pricing strategies and revenue models may need to be carefully developed and frequently reviewed to ensure they remain competitive and sustainable while offering value to users.

The most direct source of revenue may be in the form of subscription fees collected from users who subscribe to the service. Such subscriptions may be offered at various levels, with each level offering access to different features or services. For example, some embodiments of the systems and methodologies disclosed herein may feature a basic package with limited features, and premium packages that offer more personalized services or extra features. Subscription fees may be charged, for example, on a monthly, quarterly, annual, or one-time basis.

The service provider may also sell PBM devices that are designed to work optimally with the system. Such device sales may provide a significant source of revenue, especially if they are sold at a profit margin. Customers may also purchase replacement parts, upgrades, or newer models through the platform. In some embodiments, the cost of the PBM device may be subsumed in the subscription fee. This may allow for continual updating of PBM devices, while providing a regular revenue stream to the service provider.

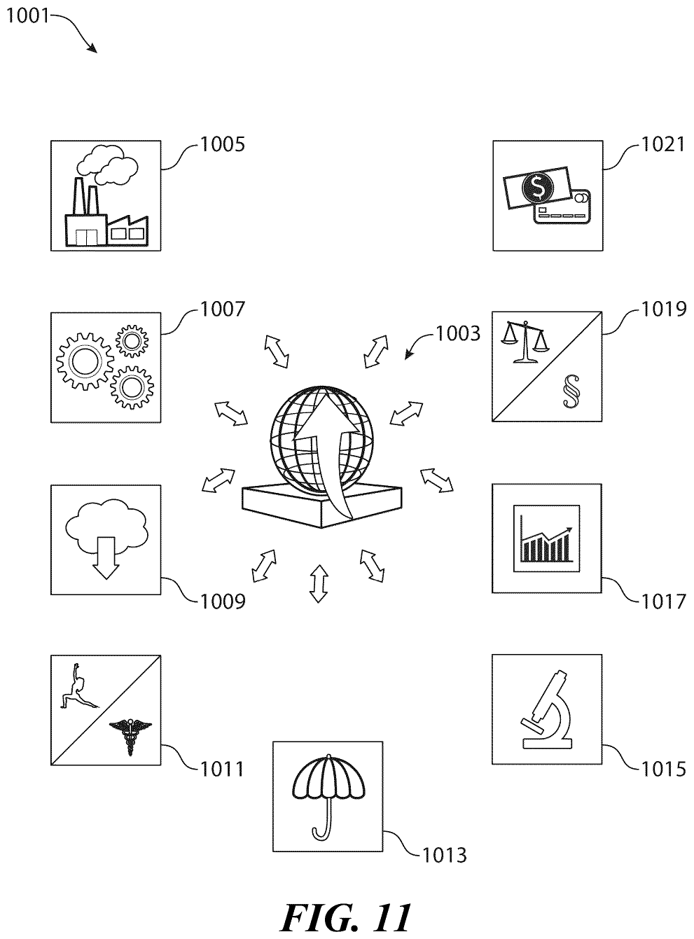

The service provider may also collaborate with wellness product manufacturers, health insurance companies, wellness clinics, or health professionals (see FIG. 11, described below). Through these partner collaborations, the provider could offer special deals, product bundles, or insurance incentives, and earn a commission on any transactions.

The platform may also offer certain premium features or content that users can access for an additional fee. These paid features may include, for example, access to specialized wellness courses, one-on-one consultations with wellness experts, or advanced wellness tracking and reporting features.

Depending on the business model, the service provider may also generate revenue from advertising. This may involve partnering with relevant businesses to display their advertisements or sponsored content on the platform. Of course, it will be appreciated that such advertising may need to be carefully implemented to maintain user trust and privacy.

Data monetization may also be a significant source of revenue for the service provider. Anonymized and aggregated data generated by the platform may be valuable for research and development in the wellness and health sector. Selling or licensing this data to research institutions or businesses, while strictly adhering to privacy laws and user agreements, could provide additional revenue streams.

The service provider may also offer corporate or institutional packages where these entities may purchase subscriptions in bulk for their employees as part of their wellness programs. Such corporate/institutional wellness programs may be a significant revenue stream if the platform demonstrates a positive impact on employee health and productivity.

It will be appreciated that various entities or parties may be involved in the implementation of the subscription based services disclosed herein. A particular, non-limiting example of the resulting financial ecosystem 1001 is depicted in FIG. 11. As seen therein, in addition to the platform service provider 1003 itself, these parties or entities may include, without limitation, device manufacturers 1005, software developers 1007, cloud service providers 1009, wellness experts and medical professionals 1011, payment processors 1021, marketing and sales partners 1017, legal and regulatory advisors 1019, insurance companies 1013, and research organizations 1015. The possible roles of these entities or parties is described in greater detail below.

The service provider 1003 is the primary entity that operates the subscription service. They are responsible for developing and maintaining the platform, managing subscriptions, and ensuring high-quality user experience.

The device manufacturers 1005 produce the PBM devices used in the system. They may be partners of the service provider or may be owned by the service provider themselves. They handle the manufacturing, quality control, and, in some cases, the direct sale of the devices to users.

Software developers 1007 may be engaged to create and maintain the platform, app, and any other necessary software. In the event that the service provider does not have an in-house software development team, they may contract an external software development company. The software developers 1007 may include or involve data scientists and AI experts for the analysis and interpretation of collected wellness data.

Since the subscription service described herein preferably relies on cloud services to store and manage data, run the AI engine, and handle communication between devices and the server, engagement of a suitable cloud service provider 1009 may be necessary. Various entities are known to the art which may be potential partners for this role including, for example, Amazon Web Services (AWS), Google Cloud, or Microsoft Azure.

Various wellness experts and medical professionals 1011 may be involved in the subscription service disclosed herein. These individuals or entities may provide professional input or validation for the personalized recommendations made by the system. They may also be involved in creating wellness content, answering user queries, or consulting on treatment plans.

Various payment processors 1021 may be involved in the subscription service disclosed herein to handle subscriptions and other financially related transactions. Various entities are known to the art which may be potential partners for this role including, for example, companies such as PayPal, Stripe, or traditional banks.

Various marketing and sales partners 1017 may be involved in the subscription service disclosed herein. These entities would be responsible for promoting the subscription service, acquiring new customers, and retaining existing ones. These may include marketing agencies, search engine optimization (SEO) consultants, or sales partners with a wide reach in the wellness sector.

Given the medical nature of the service, various legal and regulatory advisors 1019 may be required. These advisors may help to ensure that the service complies with health regulations, privacy laws, and other legal requirements in the markets they operate.

Various insurance companies 1013 may be involved in the subscription service disclosed herein. These entities may provide coverage for the PBM devices or service health plans that include the wellness service. They may also be partners providing special deals or incentives to users.

Various research organizations 1015, such as universities, research centers or research institutions, may be involved in the subscription service disclosed herein. These entities may collaborate with the service provider to conduct research on the effectiveness of PBM therapy, thereby contributing to the continued development of the service.

Various embodiments of the systems and methodologies disclosed herein may employ or leverage virtual machines (VMs). The use of VMs may provide numerous benefits in the context of a cloud-based wellness system with multiple PBM devices, contributing to a robust, flexible, and secure system. Such embodiments may leverage VMs for various purposes including, for example, providing scalability and flexibility, facilitating software testing and deployment, implementing security, aiding in disaster recovery, and establishing training and certification programs. Each of these features is described in greater detail below.

Regarding scalability and flexibility, in some embodiments, the PBM wellness system server or servers may be hosted on VMs. This may allow for easy scaling up and down as the demand for the service changes. For example, if there is a surge in users during certain times of the day or year, additional VMs may be quickly spun up to handle the increased load. Similarly, during periods of lower demand, VMs may be shut down to conserve resources.

Regarding software testing and deployment, in some embodiments of the systems and methodologies disclosed herein, VMs may be used in the development and testing of the software client and other system components. For example, developers may use VMs to create isolated testing environments that mimic the production environment, thus helping to catch and address issues before they affect the actual users. VMs may also be used to roll out updates to the software client. By testing the updates in a VM first, any problems can be caught and addressed before the update is deployed to all the PBM devices.

Regarding security, in some embodiments of the systems and methodologies disclosed herein, VMs may be used to enhance the security of the system. For instance, sensitive tasks, such as the handling of wellness data and payment processing, may be isolated in separate VMs. This way, even if one part of the system is compromised, the damage can be contained. Moreover, in some embodiments, VMs may be configured to be stateless (meaning they do not store any data between sessions). The use of such stateless VMs may help protect user data in case a VM is compromised.

Regarding security, in some embodiments of the systems and methodologies disclosed herein, VMs may also be used for disaster recovery. For example, in some embodiments, regular snapshots of the VMs may be taken and stored offsite. In case of a disaster, these snapshots may be used to quickly restore the system to a functioning state.

Regarding security, in some embodiments of the systems and methodologies disclosed herein, VMs may be used to provide hands-on training environments for the certification programs for healthcare professionals. In such embodiments for example, each trainee may have access to their own VM, allowing them to experiment with the system in a controlled environment without affecting the actual production system.

Various embodiments of the systems and methodologies disclosed herein may employ or leverage software containers. In some embodiments, such containers may be a critical part of the deployment of cloud-based wellness systems of the type described herein. Containers provide a way to wrap up an application with all of its dependencies and libraries it needs to run, in a package that can be moved and deployed easily. In the context of wellness systems, containers may be used, for example, for the application servers that process and analyze data from the PBM devices, the database that stores the wellness data, and the services that generate personalized treatment recommendations. Containers may also be used for any web servers or API servers that serve the user interface or handle communication with the PBM devices. In each case, containers may help to ensure that the services are isolated, secure, scalable, and can be efficiently updated or replaced as needed. In particular, the software containers described herein may be utilized to implement microservices architectures, ensure environmental consistency and scalability, facilitate isolation and the use of Continuous Integration/Continuous Deployment (CI/CD), and ensure the efficient use of system resources. Each of these items is described in greater detail below.

Regarding microservices architecture, in some embodiments of the systems and methodologies disclosed herein, the wellness system may be broken down into several microservices each running in its own container. These may include, for example, services for handling user registration, device communication, data analysis, and recommendation generation. This separation can make the system more manageable, scalable, and resilient.

In a microservices architecture, the application is broken down into a collection of loosely coupled services, each running in its own process and communicating with lightweight mechanisms, often an HTTP-based application programming interface (API). These services may be built around business capabilities. Preferably, each service is relatively small and may be updated, deployed, and scaled independently. Each of these microservices may run in its own container (or possibly multiple containers) for scalability. They may communicate with each other through APIs, and potentially with a central database for storing and retrieving data. With a microservices architecture, the cloud-based wellness system may be more easily managed, scaled, and updated to adapt to changing requirements or to introduce new features.

In a preferred embodiment, the implementation of a microservices architecture using containers involves service isolation, deployment, scalability and consistent environments. In particular, each microservice of the wellness system may run in its own container. This ensures that each microservice operates in isolation from the others. Consequently, if one service fails, it doesn't directly affect the other services. Similarly, with each service independently deployed as a container, if a new version of a service is ready to be released, a new container can be built and deployed to replace the old one, with minimal impact on the other services. If a particular service is experiencing high demand, additional containers for that service may be launched to handle the load. This allows for very fine-grained control over resource allocation. Containers may also encapsulate the service and its dependencies into a single deployable unit, which ensures that the service runs in a consistent environment, regardless of where the container is deployed.

In the context of the cloud-based wellness system, the application could be broken down into several microservices such as, for example, user management services, device communication services, data analysis services, recommendation services, and reporting services. Here, user management services may be used to handles user registration, authentication, and profile management. Device communication services may be used to handles communication with the PBM devices. Data analysis services may be employed to analyzes wellness data to generate personalized treatment recommendations. Recommendation services may be utilized to manage the generation and distribution of the personalized recommendations. Reporting services may be employed to handle the creation and dissemination of reports and visualizations of a user's wellness data over time.

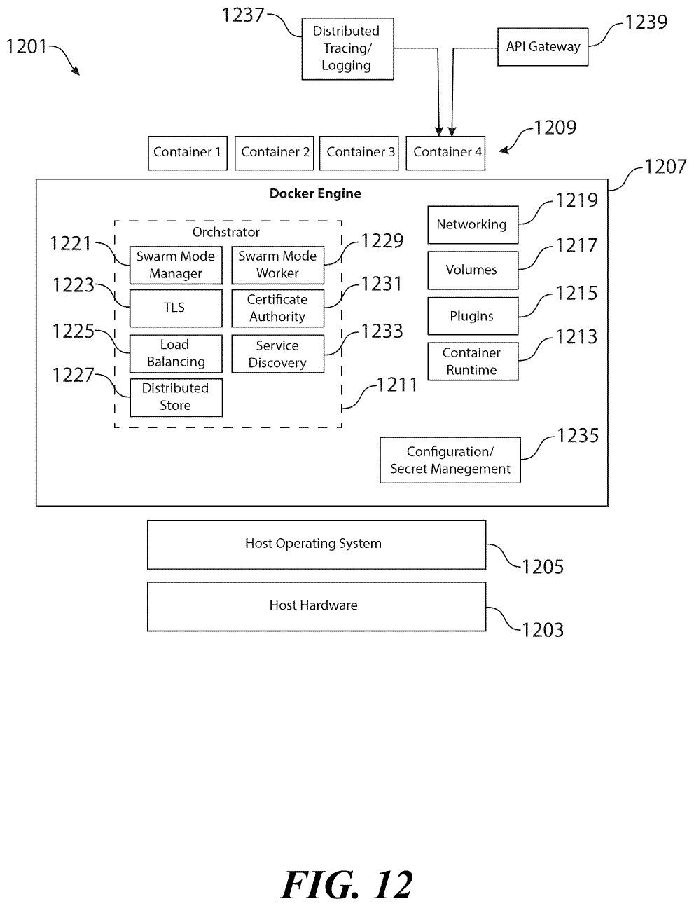

The implementation of a microservices architecture using containers in accordance with the teachings herein may involve both hardware and software layers. The implementation of a microservices architecture using containers in this manner may achieve a highly decoupled system with services that may be updated, scaled, and managed independently. FIG. 12 depicts a particular, non-limiting embodiment of a system for realizing such an implementation.

The system 1201 depicted therein comprises a hardware layer 1203, an operating system layer 1205, a Docker engine 1207, a plurality of microservices containers 1209, a container orchestrator 1211, container runtime 1213, plugins 1215, volumes 1217, networking 1219, a swarm mode manager 1221, TLS 1223, a load balancer 1225, a distributed store 1227, a swarm mode worker 1229, certificate authority 1231, service discovery 1233, a configuration and secret management module 1235, a distributed tracing and logging module 1237, and an API gateway 1239. These features are described in greater detail below.

The hardware layer 1203 is a physical layer, which may consist of multiple servers located in a data center or which may be a cloud-based infrastructure provided by services such as, for example, AWS, Azure, or Google Cloud Platform. It provides the computational resources (such as, for example, CPU, memory, storage, and networking) required to run the containers.

The operating system layer 1205 is disposed on top of the hardware layer. The operating system layer 1205 may be any suitable OS that supports containerization such as, for example, a Linux distribution (Ubuntu, CentOS), Windows Server, or a specialized lightweight OS designed for containers such as CoreOS, RancherOS, or VMware's Photon OS.

The Docker engine 1207, which forms the core of the Docker platform, is a lightweight, open-source runtime that allows developers to build and run applications inside containers. The Docker Engine 1207 is a client-server application that is responsible for all the actions related to containers such as, for example, building, pulling, running, and pushing Docker images.

The Docker Engine 1207 consists of three major components: the Docker daemon, the Docker client and the Docker API. The Docker daemon (dockerd) is a persistent process that manages Docker containers and handles container objects on the host machine. The daemon listens for requests sent via the Docker Engine API. The Docker daemon runs on the host machine.

The Docker client (docker) allows users to communicate with Docker through the command-line interface (CLI). When a user issues commands such as docker run, the client sends these commands to dockerd, which carries them out. The Docker client can communicate with more than one daemon.

The Docker API allows for programmatic access to the Docker engine. It defines the interface that programs can use to talk to the Docker daemon and instruct it what to do. Both Docker CLI and Docker Compose use this API to communicate with the Docker daemon.

The Docker Engine 1207 supports two types of runtime, a Linux runtime and a Windows runtime. The Linux runtime is based on containerd and runc, and the Windows runtime is based on the Host Compute Service (HCS) provided by Windows itself. In the context of Docker, when images are built, a network is created, or any operations are performed on containers, the Docker Engine implements all of the underlying work.

Docker containers 1209 are lightweight, standalone, and executable packages that encapsulate everything needed to run a piece of software, including the code, a runtime, libraries, environment variables, and configuration files. They provide a consistent and reproducible environment for applications, which can be easily built, shipped, and run anywhere, irrespective of the host operating system.

The orchestrator 1211 is a tool that automates the deployment, scaling, networking, and management of containerized applications. Orchestration makes it possible to manage containers that run applications, services, and their dependencies across multiple hosts. The orchestrator 1211 performs the functions of service discovery (allowing containers to find and communicate with each other), load balancing (distributing network traffic across multiple containers to ensure no single container becomes a bottleneck, thus improving user experience by improving availability of services), health monitoring (monitoring the health of containers and replacing container instances that fail), scaling (adjusting the number of containers based on the workload), rolling updates and rollbacks (gradually rolling out changes to a service or software to limit the impact of any one change, and enabling rollback of a previous deployment when something goes wrong), and secret and configuration management (managing sensitive data and configuration details for an application separate from the container image to keep sensitive data out of the stack configuration). Some of these functions are described in greater detail below. Examples of possible orchestrators which may be utilized in the systems and methodologies disclosed herein include, for example, Kubernetes, Docker Swarm, and Apache Mesos. These orchestrators may be utilized to ensure the reliability, scalability, and efficiency of containerized applications.

The container runtime 1213 is responsible for running the containers. Preferably, the container runtime 1213 is a runtime such as Docker and is installed on the OS. The container runtime 1213 is responsible for the lifecycle management of containers including, for example, the creation, starting, stopping, and destruction of container instances.

Plugins 1215 extend the core functionality of the platform, which in the presently depicted embodiment is Docker. Plugins 1215 may be effectively utilized to provide customized networking or storage, or to extend the capabilities of the platform in other ways. For example, a plugin 1215 may be utilized to store Docker volumes on a cloud-based block storage system or to add specialized networking or security capabilities. In Docker, plugins 1215 may be installed and managed like any other Docker objects.

Volumes 1217 may be utilized for persisting data which is generated and used by the containers 1209. Unlike the writable layer of a container which persists only until the container 1209 is deleted, the data in the volumes 1217 persists even after the associated container is deleted.

The platform (here, Docker) creates and manages network interfaces 1219 for the containers 1209. Docker provides different networking options to adapt to the needs of an application while ensuring isolation and security.

The swarm mode manager 1221 node is responsible for managing the swarm cluster, which involves maintaining the cluster state, scheduling services, and serving as the primary point of interface for platform commands. It also participates in the Raft Consensus Algorithm to maintain a consistent internal state across all other manager nodes in the cluster.

The orchestrator 1211 (here, Docker Swarm) uses mutual transport layer security (mTLS) 1223 to secure communications between nodes. Each node has a certificate that it uses to authenticate its communications with other nodes, ensuring that both the identity of the other party and that the communication is encrypted. This provides strong security against, for example, man-in-the-middle attacks, eavesdropping, and tampering.

Docker Swarm includes a built-in load balancer 1225 that distributes service tasks evenly across all worker nodes. It ensures that all tasks associated with a service get an even spread of network traffic. When a service is exposed to external networks, the Swarm load balancer 1225 assigns it a published port to which external traffic is routed.

Docker Swarm uses the Raft Consensus Algorithm to manage a distributed store 1227 that maintains the state of the swarm, including service definitions and network configurations. This state is distributed across all manager nodes in the swarm. If a manager node goes down, another manager node can take over with the latest consistent state.

Worker nodes 1229 in Docker Swarm serve as the workhorses of the orchestrator and are controlled by manager nodes. When a manager node assigns a task to a worker node 1229, the worker node 1229 executes the task, which involves running a Docker container. Worker nodes 1229 only run tasks and do not participate in the Raft consensus group, thereby ensuring a separation of responsibilities and reducing the complexity of the system.

As previously noted, in a preferred embodiment of the systems and methodologies disclosed herein, each microservice of the application runs in its own container and hence, such systems and methodologies have a plurality of microservices containers 1211 associated with them. A container packages up the code and all its dependencies so an associated application runs quickly and reliably from one computing environment to another. Each service (such as, for example, user management, device communication, data analysis, recommendation service, and reporting service) in the wellness systems disclosed herein may run in its own container (or containers).

Docker Swarm includes an internal certificate authority 1231 that issues certificates to nodes when they join the swarm. These certificates are used to implement mutual TLS 1223, allowing nodes to securely identify each other. The certificate authority 1231 automatically rotates these certificates on a configurable schedule to maintain security.

Docker Swarm uses a DNS component for service discovery 1233. When a service is created, it is assigned a unique DNS name and an IP address. Other services can use this DNS name to access the service. Docker's embedded DNS server maintains a mapping between service names and their IP addresses, ensuring that if a service's IP address changes, the DNS name will still resolve to the correct address.

The services discovery and load balancing functionalities of the platform may be crucial in a microservices architecture with multiple instances of services running, and tools are available which perform one or both of these functions. For example, tools such as Istio or Linkerd, or built-in service discovery and load balancing features in orchestration tools such as Kubernetes, may be used for these purposes.

The configuration and secret management module 1235 is an important component of a system that helps in securely managing configuration data and secrets (such as, for example, passwords, API keys, or SSL certificates). Here, configuration management refers to the process of handling changes in a systematic way so that a system maintains its integrity over time. In the context of Docker or any other system, configuration management typically handles the way service configurations are stored, updated, and accessed. Docker allows the configuration parameters of applications to be externalized by using environment variables or using configuration files that can be injected into a container at runtime. Secret management refers to the process of protecting secrets and other sensitive data. This data must be protected both in transit and at rest. Docker Swarm includes a built-in secret management system that allows sensitive data to be stored securely and then distributed only to the services that need it. The secrets are encrypted during transit and at rest in a Swarm, and are only accessible to the services running in the Swarm that have been specifically given access to them.

The configuration/secret management module 1235 is important for maintaining the security and integrity of an application. It allows developers to separate configuration and sensitive data from the code, reducing the likelihood of accidental exposure, while also allowing changes to the configuration and secrets to be managed in a controlled and secure way. The configuration and secret management module 1235 may be utilized for managing configurations and secrets which should not be packaged inside the containers. This module may incorporate or leverage various systems or features for this purpose including, for example, systems such as etcd, Consul, Vault, or built-in Kubernetes features.

The distributed tracing and logging module 1237 traces requests across service boundaries and centralized logging to understand behavior and to troubleshoot any issues. The use of such a module may be essential in embodiments of the wellness systems described herein, which may feature multiple, interacting services. The distributed tracing and logging module 1237 may incorporate or leverage various tools for this purpose. These include, without limitation, tools such as Jaeger or Zipkin for distributed tracing, or Elasticsearch, Logstash, Kibana (ELK stack) or Fluentd for logging.

The API gateway 1239 acts as a single point of entry into the system and is responsible for routing requests to appropriate microservices, composition, protocol translation authentication, SSL termination, and rate limiting. It also handles tasks such as load balancing, caching, access control, API metering, and logging, which can be useful for controlling and monitoring microservice interactions. Tools such as Kong, Apigee, or AWS API Gateway may be utilized for this purpose. Although the API Gateway 1239 is typically not a component of the Docker Engine 1207, it is a common component in microservices architectures and is often present in the architecture of applications that are run in Docker containers. Docker can be used to deploy and run an API Gateway, just like any other application or service. Additionally, Docker Swarm or Docker Compose can be used to orchestrate multiple services, including an API Gateway, in a microservices architecture.

Regarding environment consistency, in some embodiments of the systems and methodologies disclosed herein, containers may be leveraged to ensure that the application runs the same way in every environment, from a developer's laptop to the production servers. The use of containers here may help to achieve environmental consistency across development, testing, and production environments, which may be crucial for reliable, smooth operations and reduced debugging time, and may prevent bugs that occur when an application works in one environment but not in another. By using containers in this manner, the PBM wellness promotion systems disclosed herein may ensure environmental consistency across different stages of development and deployment, leading to fewer bugs, increased productivity, and quicker deployment times. Below is a detailed description of how containers may be utilized to achieve environmental consistency in the PBM wellness promotion systems disclosed herein.

One of the preliminary steps in the utilization of containers in the systems and methodologies disclosed herein is the process of containerization. The principal concept underlying this step is the bundling of an application along with all of its related configuration files, libraries, and the dependencies required for it to run in an efficient and bug-free way across different computing environments. This may be achieved, for example, by creating a container image, which is a lightweight, stand-alone, executable package. In the case of the wellness systems described herein, each microservice (such as, for example, user management, device communications, data analysis, recommendation services, or reporting services) may be packaged into its own container image.

Once a container image is created, it is immutable (that is, it does not change). This ensures that the same image may be utilized in development, testing, and production environments, and it will behave in the same way. This avoids the common problem of code working in development but failing in production due to environmental differences.

The container images may be version controlled, in a manner similar to that utilized for source code. When a new version of an application is ready for deployment, a new container image may be created and tagged with a version number. Old versions are still available and may be run if needed, thus facilitating rollback in case of problems.

Containers run in isolation from each other, even though they share the host system's kernel. Consequently, they have their own resources including, for example, their own filesystem, CPU, memory, and process space. This isolation helps to ensure that any changes in one container do not affect other containers, and also aids in maintaining environmental consistency by avoiding conflicts between different service dependencies.

The configuration of the application environment may be defined and managed using code with container orchestration tools such as, for example, Kubernetes. This code may be version controlled and consistently applied across all environments, thus helping to maintain environmental consistency.

The use of containers in the systems and methodologies disclosed herein allows the process of deploying their associated applications to be repeatable and readily automated using, for example, Continuous Integration/Continuous Deployment (CI/CD) pipelines. This repeatable deployment process helps to maintain environmental consistency, since the same deployment process is used every time.

The use of containers in the systems and methodologies disclosed herein also facilitates horizontal scaling by running multiple instances of the same container. For example, load balancers may be used to distribute network traffic evenly across these instances, thus ensuring consistency of service even under high load.

Regarding scalability, in some embodiments of the systems and methodologies disclosed herein, a container-based approach may be utilized to allow the wellness system to scale its services up or down to meet the demand, ensuring that users receive consistent and responsive service. The use of containers also supports automated scaling strategies, which can dynamically adjust resources in real-time based on system load and predefined policies. Using this approach, scalability may be achieved by deploying multiple instances of the same service to handle increasing loads. Containers may be rapidly started or stopped, which may facilitate scaling applications. For example, if the demand for a particular service increases, additional containers running that service can be started.

In operation, as the demand on a particular service increases, the container orchestrator 1211 (see FIG. 12) can spin up additional instances of the corresponding container to handle the load, providing horizontal scaling. For example, if the container orchestrator 1211 is Kubernetes, then the load balancing features of Kubernetes ensure that traffic is evenly distributed among the instances of a service, preventing any single instance from becoming a bottleneck. Simultaneously, Kubernetes continuously monitors the health of all running containers, and if it detects a problem with a particular instance, it can automatically replace it with a new one, improving system reliability.

Regarding isolation, in some embodiments of the systems and methodologies disclosed herein, each container may be isolated from other containers and from the host system. In such embodiments, bugs or security vulnerabilities in one service will not propagate to other services. This is especially important in wellness systems where sensitive personal health data is handled. Suitable containerization technologies (such as, for example, Docker) may be utilized to achieve isolation in the wellness promotion systems disclosed herein. Containers allow developers to encapsulate an application and its dependencies into a single self-contained unit that can run anywhere, providing process isolation and enabling consistent behavior across different environments. A container-based system also enables process and resource isolation, protecting services from each other, while ensuring consistent behavior across various environments. This level of isolation can significantly enhance the security and reliability of the wellness promotion system.

Regarding Continuous Integration/Continuous Deployment (CI/CD), in some embodiments of the systems and methodologies disclosed herein, containers may be integrated into a CI/CD pipeline. In such a pipeline, developers may build and test application code within containers, which may then be pushed to testing or production environments upon completion. CI/CD is a DevOps practice where developers regularly merge their code changes into a central repository, after which automated builds and tests are run. CI/CD is complemented by Continuous Deployment, a strategy for software releases wherein every code commit that passes the automated testing phase is automatically released into the production environment, making changes that developers make to the software functional and visible to users in real-time. Containers may play a significant role in facilitating CI/CD, promoting efficiency, consistency, and scalability.

In the context of the wellness systems described herein, CI/CD may be achieved by a process featuring a code repository, continuous integration, container registry, continuous deployment, configuration management, and monitoring/logging. Each of these items is described in greater detail below. In a typical implementation, developers working on the various microservices (for example, user management services, PBM device communication services, data analysis services) of the wellness system commit their code to a version control system such as Git. The use of such a code repository allows for the management of code changes and history tracking.

In order to achieve continuous integration, CI tools (such as, for example, Jenkins or Travis CI) may be utilized. These tools are configured to automatically build and test new code committed to the code repository. Each microservice's code is pulled, dependencies are installed, and the application is packaged into a container. Automated tests are then run on these containers to ensure that the changes do not break the application. If the build and tests pass, the CI system pushes the container image to a registry (such as, for example, Docker Hub or Google Container Registry). This image includes the application and all its dependencies, ensuring consistency when the application is deployed to various environments.

In the CD phase, tools such as Kubernetes or Docker Swarm are utilized to pull the latest container image from the registry and deploy it to the appropriate environment (staging or production). This may be achieved through rolling updates, which allow for zero-downtime deployments. Tools such as Ansible, Chef, or Puppet may be utilized to manage the configuration of the containerized applications and the underlying infrastructure. This helps to maintain consistency across different deployment environments. Once deployed, the applications may be monitored using tools such as, for example, Prometheus or ELK Stack. These tools collect logs and metrics from running containers to detect any issues and enable quick troubleshooting. In the foregoing manner, containers facilitate CI/CD by providing a consistent environment from development to production, helping to find and fix issues quickly, ensuring that software is always in a releasable state, and enabling developers to respond quickly to changes in requirements.

Regarding the efficient use of system resources, in some embodiments of the systems and methodologies disclosed herein, containers are lightweight solutions that share the host system's OS kernel. As a result, they are able to start quickly and use fewer system resources than comparable virtual machines. Containers can significantly improve the efficiency of system resource usage by providing on-demand provisioning, detailed resource management, dynamic scaling, and service consolidation. The use of containers in a microservices architecture of the type disclosed herein provides flexibility and control over resource allocation, improves system reliability, and enables scalable responses to varying demand.

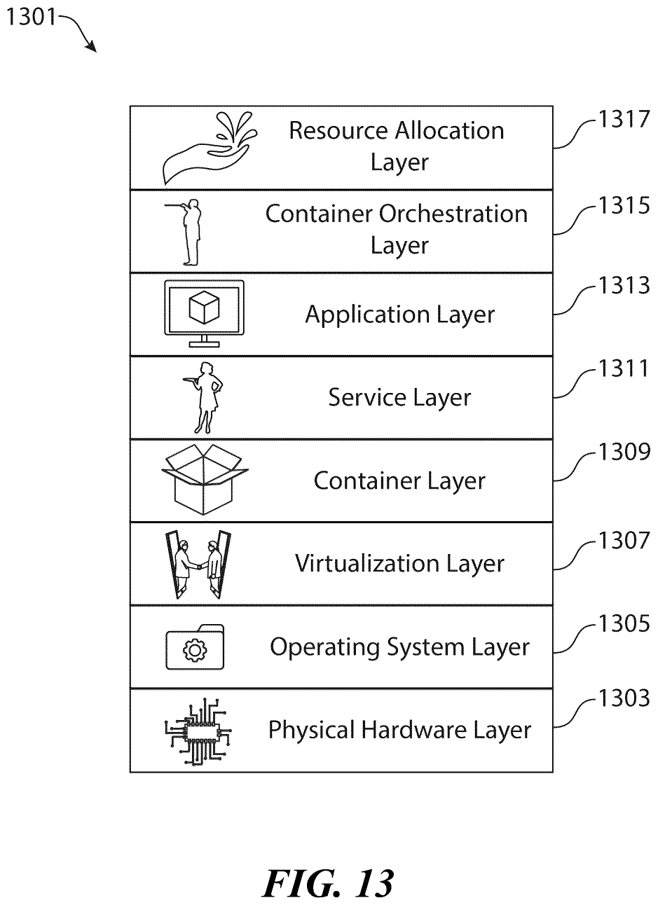

FIG. 13 depicts a particular, non-limiting embodiment of the architecture of a containerized system of the type disclosed herein. As seen therein, the system 1301 comprises a physical hardware layer 1303, an operating system layer 1305, a virtualization layer 1307, a container layer 1309, a service layer 1311, an application layer 1313, a container orchestration layer 1315, and a resource allocation layer 1317. A layered architecture of this type allows for a high level of modularity and flexibility. In particular, each layer in the system 1301 may be managed and scaled independently, and changes in one layer generally have minimal impact on the others. This makes the system 1301 robust and adaptable, capable of serving a wide range of application needs in different environments.

The base of the system 1301 is the physical hardware layer 1303, which may include multiple servers located in a data center or cloud-based servers. These servers have underlying physical resources such as CPU cores, memory, and storage that can be used by the hosted applications. Each of these servers runs an operating system (OS) in the OS layer 1305 which manages the hardware resources and provides services for the software. The OS hosts the container runtime, such as Docker, which allows containers to share the OS kernel and run independently of each other.

A virtualization layer 1307 is disposed over the OS layer 1305. The virtualization layer 1301 includes a container orchestration system such as, for example, Kubernetes or Docker Swarm. These orchestrators allocate resources to containers and manage the lifecycle of the containers running on the servers.

In the container layer 1309, different parts of the wellness system are divided into microservices, each encapsulated within its container. For example, there might be separate containers for user management, device communication, data analysis, treatment recommendations, and so forth. Each container has its runtime environment and dependencies packaged within, reducing potential conflicts and enhancing reliability.

The service layer 1311 interfaces with the containers, receiving user requests and distributing them to the appropriate containers. It also manages the responses from the containers, presenting them back to the user or other services.

The application layer 1313 includes the front-end user interfaces by which users interact with the system. These front-end user interfaces may include, for example, a website or a mobile application. The application layer 1313 communicates with the service layer 1311 to provide functionality for the users.

The container orchestration layer 1315 includes a container orchestration system that monitors the load on each service and automatically scales the number of containers for each microservice up or down based on demand. This dynamic scaling allows for efficient use of system resources, only using what is needed at a given time. With containerization, the system administrator can limit the amount of system resources that a container can use. This may include, for example, setting a limit on the amount of CPU or memory a particular container can use. This can prevent a single container from using all available resources and negatively affecting other containers.

The resource allocation layer 1317 provides the means by which the system admin can limit the amount of system resources that a container can use. This may include, for example, setting a limit on the amount of CPU or memory a particular container can use. This may prevent a single container from using all available resources and negatively affecting other containers.

The PBM devices disclosed herein may be equipped with their own operating systems to facilitate their use in a wellness system. The operating system is preferably lightweight and efficient, due to the limited computational resources that may be available to the PBM device. The operating system is also preferably equipped to support networking for communication with other devices and the cloud. Embedded operating systems such as, for example, FreeRTOS, Embedded Linux, or a lightweight version of Android, may be suitable for some embodiments of such a device. The use of embedded Linux operating systems is preferred.

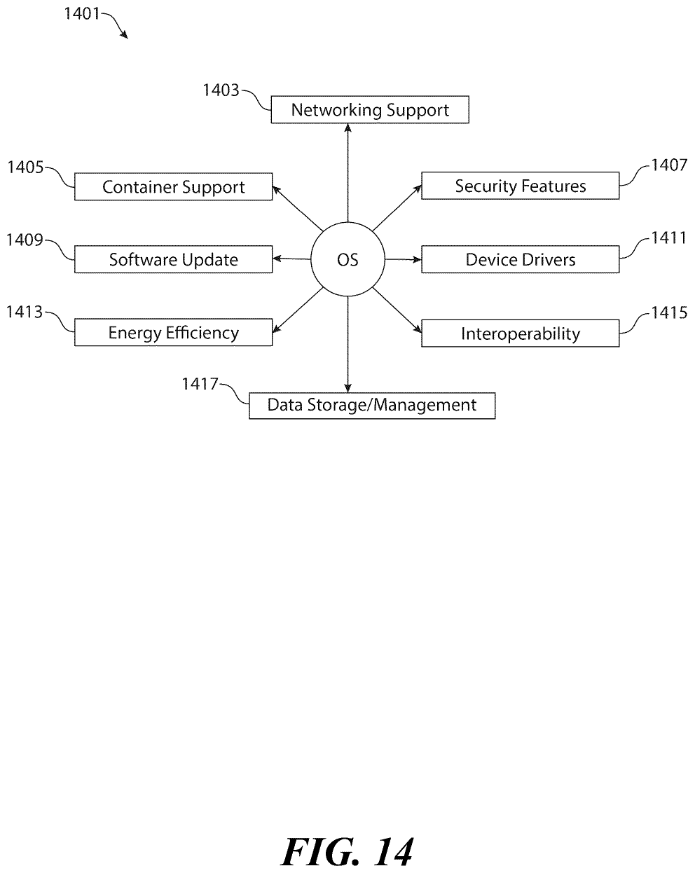

FIG. 14 depicts a particular, non-limiting embodiment of a Linux operating system in accordance with the teachings herein which may be utilized for this purpose. The operating system 1401 depicted therein is equipped with networking support 1403, container support 1405, security features 1407, a software update mechanism 1409, device drivers 1411, an energy efficiency module 1413, interoperability capability 1415, and data storage and management 1417. By adding these features to an embedded operating system such as Linux, a PBM device may be produced which is capable of supporting complex, cloud-based wellness systems of the type described herein. These features are described in further detail below.

In terms of networking support 1403, the operating system should have a networking stack that supports both wired (such as, for example, Ethernet) and wireless (such as, for example, Wi-Fi or Bluetooth) communications to transmit and receive data. This enables the PBM device to communicate with other devices and the server.

Regarding container support 1405, while containerization is more commonly used in server environments, lightweight container solutions such as, for example, Docker or Balena, can run on certain embedded Linux distributions. This may enable features such as isolation, environmental consistency, and efficient resource usage discussed earlier.

With respect to security features 1407, since the PBM device will be communicating wellness data, the operating system is preferably equipped with strong encryption and authentication mechanisms to protect data privacy and integrity. The device may utilize protocols such as TLS/SSL for secure data transmission. Notably, the Linux kernel has strong built-in support for various cryptographic algorithms.

The software update mechanism 1409 is preferably an over-the-air (OTA) update feature that delivers software and firmware updates to the PBM device. This feature ensures that the device has the latest features, security updates, and bug fixes.

Device drivers 1411 are software components that allow the operating system to interact with the PBM hardware. These drivers may enable the system to control and monitor the operation of the PBM device, such as starting a therapy session, adjusting intensity, selecting therapeutic wavelengths or frequencies, or monitoring device status.

Regarding energy efficiency module 1413, the operating system preferably has power management features to optimize the battery life of the PBM device. This may include putting the device into a low-power sleep mode when it's not actively delivering treatment or communicating data.

In terms of interoperability 1415, the operating system preferably supports standard communication protocols to ensure that the PBM device can communicate with other devices and platforms. This preferably includes standard internet protocols such as, for example, TCP/IP and HTTP, as well as IoT-focused protocols such as MQTT.

With respect to data storage and management 1417, the operating system preferably manages local data storage to record treatment history and device usage. This may involve a lightweight database system or a simple file-based system.

The foregoing operating system may be readily adapted to function in a Web3 (or Web 3.0) environment. Such an environment is often associated with the decentralized internet, where applications run on peer-to-peer networks, and data is controlled and owned by the users rather than centralized servers. Blockchains, distributed ledgers, and decentralized applications (dApps) are key elements of the Web3 paradigm. The transition of the operating system of the PBM device to function in a Web3 environment may require some additions and modifications to the operating system. Adopting these changes may allow the operating system of the PBM device to fully participate in a Web3 environment, empowering users with greater control over their data and facilitating decentralized operation of the wellness system. These modifications and additions are described in greater detail below.

One modification may be the addition of support for blockchains and distributed ledgers. In particular, in order to interact with blockchains or distributed ledgers, the operating system may need to incorporate or communicate with appropriate software clients or nodes. This may involve running light clients that can interact with the blockchain without downloading the full transaction history or communicating with external full nodes.

A further modification may be smart contract integration. In particular, in order to interact with dApps, the operating system will typically need to support smart contract execution. Smart contracts are self-executing contracts where the agreement's terms are directly written into code. These can facilitate, verify, or enforce the negotiation or performance of a contract in a blockchain context.

In a Web3 environment, users are able to control their digital identities through Decentralized Identifiers (DIDs). The OS will typically need to support the generation, storage, and management of these identifiers, along with their associated cryptographic keys. In addition, since data in Web3 is often stored in decentralized file systems such as InterPlanetary File System (IPFS) or Filecoin, the OS will typically need to support integration with such decentralized storage systems, thus allowing it to store and retrieve data in a decentralized manner.

The operating system will also typically need to engage in peer-to-peer (P2P) communications. This typically requires support for P2P protocols to facilitate direct communication between devices. Such P2P communication differs from the communications that occur in a traditional client-server model and allows for decentralized communication.

In the decentralized Web3 environment, privacy and security are typically paramount. Accordingly, the operating system will typically need to be equipped with suitable security and privacy enhancements. These will preferably include advanced cryptographic functionalities for secure communications, transactions, and data storage.

The operating system should have built-in token/cryptocurrency support for handling digital assets or tokens. Such digital assets or tokens are often used for transactions in the Web3 ecosystem.

The use of the operating systems for PBM devices disclosed herein may be especially beneficial in an Internet of Things (IoT) setting. This is particularly true if the operating system is integrated with virtualization technologies such as containers and virtual machines. Such embodiments of the operating system offer advantages in terms of edge computing, scalability, interoperability, security/isolation, efficient resource utilization, remote management and continuous updates, and data management/privacy.

For example, it may be desirable to couple the PBM device to an edge computing device, or to have an edge computing device incorporated into the PBM device. IoT devices often operate at the edge of the network, close to where data is generated. Having a lightweight operating system with container support may facilitate edge computing by enabling computation to be done directly on the PBM device, thus reducing latency and improving the responsiveness of the system.

Such edge computing may be implemented or facilitated with various hardware. Such hardware may include, for example, edge gateways, edge nodes, micro data centers or edge servers, Field Programmable Gate Arrays (FPGAs) or Application-Specific Integrated Circuits (ASICs), SD-WAN appliances, embedded AI hardware, and secure elements. Specifically, FPGAs or ASICs are specialized hardware devices which may be programmed to carry out specific computational tasks very efficiently, and which may be incorporated into the PBM device to handle real-time processing needs. Embedded AI hardware may be utilized for AI-powered edge applications, and may include specialized AI hardware such as, for example, AI accelerators, GPUs, or Neural Processing Units (NPUs), all of which may be added to the PBM device or to a device in communication with it. These units provide the ability to process complex AI and Machine Learning algorithms at the network edge. Secure elements may include, for example, chips which are added to the PBM device to provide a secure storage and execution space for sensitive data and operations, the storage of cryptographic keys, the performance of secure booting, and the management of secure updates.

Regarding scalability, as the number of connected PBM devices in an IoT network grows, the ability to efficiently manage and scale services may become critical. The operating systems described herein, and especially those embodiments with support for containers, may quickly deploy, start, stopped, and replicate services as needed. This ability may significantly simplify the management and scalability of services across a large number of PBM devices.

Regarding interoperability, IoT networks often consist of diverse devices from different manufacturers, each potentially running different operating systems and software stacks. By employing containerization or virtualization, the operating system may ensure that the applications run consistently across various devices, regardless of the underlying hardware or software configurations. This feature may greatly enhance the interoperability of devices within an IoT network.

With respect to security and isolation, containers and VMs provide a level of isolation between different applications running on the same device. If one application or service is compromised, the effect of the compromise can be limited to that particular container or VM, helping to protect other applications and the device itself.

Regarding efficient resource utilization, containers allow multiple applications to share the same OS kernel, while keeping their execution environments separate. This may lead to more efficient resource utilization compared to running each application on a separate VM or device. For PBM devices, which may have limited computational resources, this may enable running more complex or a larger number of applications on the same device.

With respect to remote management and continuous updates, containerization and virtualization may simplify the process of managing devices and deploying updates. For example, new versions of applications or operating system updates may be packaged into a new container or VM image, and then deployed to devices with minimal downtime.

Regarding data management and privacy, with containers or VMs, data may be managed and isolated at a granular level. This may enable better privacy controls, as each application may be restricted to access only its own data, thereby reducing the risk of data leaks or breaches.

In addition to the foregoing advantages, the strategic deployment of an operating system of this type on PBM devices may offer various other advantages. For example, healthcare providers or wellness services may leverage such an operating system to build a robust, secure, and highly scalable IoT network, capable of delivering personalized, real-time PBM therapies to a large number of users.

Operating systems of the type described herein may also offer specific advantages in the context of the web-based subscription models described herein. By leveraging these advantages, the subscription service can differentiate itself in the market, providing value to users and stakeholders alike.

For example, the operating system's capabilities for personalization and real-time response, made possible through its IoT compatibility and edge computing, offers the potential to enhance user experience by enabling personalized treatment recommendations, real-time adjustments to treatment based on user feedback or sensor data, and rapid response times. Moreover, the inherent scalability provided by containerization in these operating systems allows the service to easily expand or contract based on the number of subscribers. It ensures that the performance of the service remains consistent, whether there are hundreds or hundreds of thousands of users. In addition, the use of containers and VMs also enables continuous integration and continuous deployment (CI/CD). Consequently, updates, bug fixes, and new features may be rapidly and frequently rolled out to users without interrupting service.

The environmental consistency provided by containers may also ensure that every user, regardless of the specific hardware or configuration of their PBM device, receives a consistent quality of service. This is particularly important in a subscription model, where subscribers will expect the same level of service regardless of their specific device. Moreover, the ability to offer service consistency across various models of PBM devices enables business models in which PBM devices are provided as part of the service and are frequently swapped out, much like cable modems or routers currently form part of the service offering of cable providers.

The security enhancements offered by these operating systems are also a differentiator in the marketplace. Security is a major concern in any system handling personal health data. The isolation features of containers and VMs in the operating systems disclosed herein ensure that, even in the event of a security breach, damage can be contained, and the rest of the system can be protected. This may provide peace of mind to subscribers about the security of their data.

The efficient use of resources provided by containerization means less hardware is required to serve the same number of users compared to more traditional architectures. These cost savings can be passed onto customers, making the subscription more affordable, or reinvested into improving the service.

The combination of IoT data collection and edge computing enables data-driven insights to be generated in real-time. These insights may be used to enhance the effectiveness of PBM treatments, leading to better outcomes for users and providing a powerful selling point for the subscription service.

In some embodiments of the systems and methodologies disclosed herein, PBM wellness systems may be provided which advantageously leverage communication protocols which use the principles of block chaining and hyper distribution communications protocols (such as, for example, bit torrent) to provide a highly secure network of radio nodes that are organized in a mesh topology. In some embodiments, an RF client may be utilized in these systems to implement such a protocol to provide always-on, one-to-many communications among radio nodes that may leverage full streaming network concepts such as 5G, thereby providing a highly secure, efficient, and resilient network. Such a network of radio nodes, organized in a mesh topology, can significantly improve data reliability, security, and efficiency.

A mesh network is a network topology in which each node relays data for the network. All mesh nodes cooperate in the distribution of data in the network, making it highly resilient and efficient. If one node fails, there are multiple other paths to ensure data reaches its destination.

In embodiments of this type, hyperdistribution protocols (such as, for example, BitTorrent) may be used to distribute wellness data across the network. Each PBM device (acting as a node in the network) may store only a fragment of the total wellness data, thereby reducing the storage burden on each device. This fragmented data may be encrypted for additional security.

By utilizing a blockchain-based protocol, each transaction (in this context, a data transaction from a PBM device) may be recorded in a block and added to the chain in a linear, chronological order. This provides a decentralized and immutable record of all data transactions, enhancing the security and reliability of data. The distributed ledger feature of blockchain may enable a secure and transparent system where every participant can verify the transactions.

A radio frequency (RF) client in such a system may implement this protocol to provide always-on, one-to-many communication among the radio nodes. This RF client may be a piece of hardware or a software program running on the PBM device that can manage the wireless communication between the device and the network.

The RF client may leverage 5G technology to support the high-speed transmission of wellness data. Here, it is to be noted that the use of 5G technology in this application is especially beneficial, given its high data rate, reduced latency, energy savings, cost reduction, higher system capacity, and massive device connectivity. The RF client may leverage 5G's features to enable streaming of wellness data in near real-time, which may be crucial in cases where real-time monitoring of patient data is required. This high-speed connectivity may also enable quicker analysis and feedback, allowing users to adjust their wellness plans faster.

As a specific, non-limiting example of an implementation of the foregoing system, each PBM device (node) collects wellness data from its user and encrypts it. The device breaks the data into chunks and distributes it to nearby nodes in the mesh network using the hyperdistribution protocol. As chunks of wellness data are received, the receiving nodes verify them and record the transactions into their version of the blockchain. This decentralized, transparent ledger ensures that the data has not been tampered with and provides a trustworthy record of all transactions.

When the server (or any other authorized entity) needs to access a user's wellness data, it sends a request to the network. The network nodes collaborate to provide the data chunks, which the server then pieces together to get the full data set. If any node goes offline or is compromised, the network can still function. The data is not stored in a single place, but rather distributed across the network, increasing data durability.

With an RF client leveraging 5G technology, data can be transmitted quickly and securely across the network. This enables real-time or near real-time data analysis and faster response times.

It will be appreciated that implementations such as the foregoing offer several benefits in PBM wellness systems in general, and subscription models of such systems in particular. These implementations offer networks which are more resilient, secure, and efficient in handling wellness data, and which offer the potential for enhanced user experiences and better wellness outcomes.

Various embodiments of the systems and methodologies disclosed herein may utilize hypervisors. A hypervisor, also known as a virtual machine monitor, is a piece of computer software, firmware or hardware that creates and runs virtual machines. A computer on which a hypervisor runs one or more virtual machines is called a host machine, and each virtual machine is called a guest machine. The use of hypervisors may provide several advantages in the systems and methodologies disclosed herein.

Hypervisors offer several advantages in terms of efficient resource utilization. In particular, hypervisors allow for the creation of multiple virtual machines on a single physical host. Each virtual machine may run a separate operating system and execute different tasks. This enables or facilitates the efficient use of hardware resources such as CPU, memory, and storage.

The use of hypervisors in the systems and methodologies disclosed herein also offers advantages in isolation and security. Each virtual machine in a hypervisor environment operates in complete isolation from the others. Consequently, if a security issue or a crash occurs in one virtual machine, it does not affect the others. This isolation feature enhances the security and reliability of the system.

The use of hypervisors in the systems and methodologies disclosed herein also facilitates maintenance and upgrades. For example, a new version of a service can be deployed on a new virtual machine, and traffic can be redirected to it. If any problems arise, traffic can be switched back to the previous version quickly. Hypervisors also provide an ideal environment for testing and development, since new software versions or features can be deployed and tested in a virtual machine without affecting the production environment. The use of hypervisors may also improve the scalability of the system, since new virtual machines may be quickly created and deployed as the demand for services increases.

In the context of the PBM wellness systems described herein, hypervisors may be utilized in the server infrastructure to efficiently manage and isolate various services. For example, in some embodiments of these systems, the artificial intelligence engine, the distributed ledger system, and the user interface may each run in separate virtual machines, providing both isolation and efficient use of resources. In terms of system hierarchy, in a specific embodiment, the hypervisor is at the highest level and directly interacts with the server hardware. It is responsible for managing the VMs, and each VM runs its dedicated software component. The VM hosting the AI engine accepts wellness data from the PBM devices, performs data analysis, and generates personalized treatment recommendations. The VM for the distributed ledger system manages the secure and transparent recording of wellness data and treatment recommendations. Lastly, the VM for the under interface provides an interface for users to interact with the system, receive recommendations, input their wellness goals and track their progress. Such a system hierarchy ensures that the system components are isolated from each other, enhancing security and efficiency, and allowing for better resource management and scalability.

Moreover, in the IoT environment, lightweight hypervisors could be used to run multiple lightweight operating systems on the PBM devices. This may allow the devices to perform multiple tasks or services in an isolated and secure manner.

The above description of the present invention is illustrative and is not intended to be limiting. It will thus be appreciated that various additions, substitutions and modifications may be made to the above described embodiments without departing from the scope of the present invention. Accordingly, the scope of the present invention should be construed in reference to the appended claims. It will also be appreciated that the various features set forth in the claims may be presented in various combinations and sub-combinations in future claims without departing from the scope of the invention. In particular, the present disclosure expressly contemplates any such combination or sub-combination that is not known to the prior art, as if such combinations or sub-combinations were expressly written out.

What is claimed is:

1. A method for providing coordinated wellness treatments using a plurality of PBM devices, comprising:
collecting wellness data from each PBM device;
transmitting the collected wellness data to a server;
analyzing the wellness data to generate personalized treatment recommendations;
recording the wellness data and treatment recommendations onto a distributed ledger system;
communicating the personalized treatment recommendations to each PBM device; and
adjusting the PBM treatments based on the personalized recommendations.

2. The method of claim 1, further comprising the step of using smart contracts to automate treatment adjustments based on pre-defined criteria.

3. The method of claim 1, wherein the step of recording involves the use of cryptographic methods to ensure the security and integrity of the wellness data and treatment recommendations.

4. The method of claim 1, further comprising the step of managing user permissions for data access on the distributed ledger system.

5. The method of claim 1, wherein the distributed ledger system is a blockchain that provides decentralized, tamper-resistant storage of the wellness data and treatment recommendations.

6. The method of claim 1, further comprising the step of granting secure access to the distributed ledger system to authorized healthcare providers for real-time monitoring of the wellness data and treatment recommendations.

7. The method of claim 1, wherein the recording step includes using cryptographic hashes to ensure the integrity and authenticity of the wellness data and treatment recommendations.

8. The method of claim 1, wherein the recording step includes timestamping the wellness data and treatment recommendations to create a chronological history of wellness treatments.

9. The method of claim 1, further comprising the step of using smart contracts in the distributed ledger system to automate the execution of certain predefined actions based on the wellness data and treatment recommendations.

10. The method of claim 1, wherein the communicating step includes querying the distributed ledger system to fetch the most recent treatment recommendations for each PBM device.

11. The method of claim 1, wherein each PBM device is associated with a unique digital identity in the distributed ledger system, facilitating device-specific treatment customization and data tracking.

12. The method of claim 1, further comprising the step of using the distributed ledger system to securely share the wellness data and treatment recommendations with other healthcare systems or platforms.

13. The method of claim 1, wherein the distributed ledger system is used to facilitate interoperability and data synchronization between the PBM devices, the server, and any other connected health management systems.

14. The method of claim 1, wherein the distributed ledger system is used to facilitate real-time auditing and regulatory compliance checks of the wellness data and treatment procedures.

15. The method of claim 1, further comprising the step of using consensus mechanisms in the distributed ledger system to validate new entries of wellness data and treatment recommendations.

16. The method of claim 1, wherein the adjusting step includes making changes to the treatments based on an analysis of trends or patterns in the wellness data stored on the distributed ledger over time.

17. The method of claim 1, wherein the server, PBM devices, and the distributed ledger system collectively form a peer-to-peer network for collaborative wellness management.
